(19) United States
(12) Patent Application Publication
(10) Pub. No.: US 2009/0292163 A1
(43) Pub. Date: Nov. 26, 2009

(54) DEVICES AND METHODS FOR ACHIEVING THE LAPAROSCOPIC DELIVERY OF A DEVICE

(75) Inventors: Ghassan S. KASSAB, Indianapolis, IN (US); Jose A. Navia, SR., Buenos Aires (AR)

Correspondence Address: ICE MILLER LLP, ONE AMERICAN SQUARE, SUITE 3100, INDIANAPOLIS, IN, 46282-0200, US

(73) Assignee: CVDevices, LLC, Zionsville, IN (US)

(21) Appl. No.: 12/431,505

(22) Filed: Apr. 28, 2009

Related U.S. Application Data
(63) Continuation-in-part of application No. 12/395,516, filed on Feb. 27, 2009.
Application No. 12/395,516 is a continuation-in-part of application No. 12/307,111, filed on Dec. 30, 2008.
Application No. 12/307,111 is a continuation-in-part of application No. 12/307,113.
Application No. 12/307,113 is a continuation-in-part of application No. 11/997,147, filed on Jun. 30, 2008.
(60) Provisional application No. 60/817,423, filed on Jun. 30, 2006.
(60) Provisional application No. 60/817,423, filed on Jun. 30, 2006.
(60) Provisional application No. 60/703,421, filed on Jul. 29, 2005.

(51) Int. Cl. A61B 1/00

(52) U.S. Cl. 600/104

(57) ABSTRACT

Devices and methods are disclosed for delivering an instrument laparoscopically to a targeted tissue. Embodiments of the device comprise a handle, a lift system and two arms extending therebetween and are capable of achieving the parallel closure of the instrument around the targeted tissue. Further, the methods described can be performed through entirely laparoscopic techniques, including the activation of any pins disposed on the instrument being delivered. Such devices and methods may be used to deliver an instrument to any tissue of a body.

### RELATED APPLICATIONS

This U.S. Utility patent application (1) is a continuation-in-part of U.S. patent application Ser. No. 12/395,516, filed Feb. 27, 2009, which is a nationalization of International Application No. PCT/U.S.07/15267, filed Jun. 29, 2007, which claims priority to U.S. Provisional Patent Application Ser. No. 60/817,423, filed Jun. 30, 2006; (2) is a continuation-in-part of U.S. patent application Ser. No. 12/307,111, filed Dec. 30, 2008, which is a nationalization of International Application No. PCT/U.S.07/15238, filed Jun. 29, 2007, which claims priority to U.S. Provisional Patent Application Ser. No. 60/817,423, filed Jun. 30, 2006; (3) is a continuation-in-part of U.S. patent application Ser. No. 12/307,113, filed Dec. 30, 2008, which is a nationalization of International Application No. PCT/U.S.07/15267, filed Jun. 29, 2007, which claims priority to U.S. Provisional Patent Application Ser. No. 60/817,423, filed Jun. 30, 2006; and (4) is a continuation-in-part of U.S. patent application Ser. No. 11/997,147, filed Jun. 30, 2008, which is a nationalization of International Application No. PCT/US06/029424, filed Jul. 28, 2006, which claims priority to U.S. Provisional Patent Application Ser. No. 60/703,421, filed Jul. 29, 2005. The content of each of the foregoing applications is hereby incorporated by reference in its entirety into this disclosure.

### BACKGROUND

Organ and tissue remodeling are clinical techniques that may be applied to numerous different body tissues, ranging from blood vessels to whole organs. Conventionally, such remodeling techniques require incisions and/or sutures in the tissue to be remodeled in order to alter the tissue's anatomy. For example, gastric remodeling is often employed to treat obesity and typically involves the reorganization of the digestive tract. Conventional examples of such procedures involve attempts to either 1) restricting food intake into the body via a restrictive bariatric procedure (a “Restrictive Procedure”), or 2) altering the anatomy of the small intestine or divert the peristalsis of a person's normal food intake past the small intestine to decrease caloric absorption via a malabsorptive bariatric procedure, which is commonly known as a gastric bypass (a “Malabsorptive Procedure”). It is also known to combine the two procedures such that both of the aforementioned techniques are employed jointly.

Malabsorptive Procedures entail an intestinal bypass that results in the exclusion of almost all of the small intestine from the digestive tract. In most Malabsorbptive Procedures, a portion of the stomach or small intestine is removed from the digestive tract through a surgical procedure that requires cutting the digestive tissue and thereafter closing any holes or securing the newly formed anatomy with staples and/or sutures. Conversely, Restrictive Procedures generally involve the creation of a passageway extending from the upper portion of the stomach to the lower portion of the stomach in order to decrease the size of the organ and thus prevent the stomach from storing large amounts of food. Conventional Restrictive Procedures rely on the banding and/or stapling of the stomach to create a small pouch on the superior portion of the stomach near the gastroesophageal junction.

Combined operations consisting of Malabsorptive and Restrictive Procedures are the most common bariatric procedures performed today. An example of a combined procedure is the Extended (Distal) Roux-en-Y Gastric Bypass in which a stapling creates a small (approximately 15 to 20 cc) stomach pouch completely separated from the remainder of the stomach. In addition, the small intestine is divided just beyond the duodenum (the hollow tube connecting the stomach to the jejunum), re-arranged into a Y-configuration, and sutured to the small upper stomach pouch to enable the outflow of food therefrom through the newly formed “Roux limb.”

Accordingly, most digestive tract remodeling procedures require that the stomach and/or tissue of the intestine is cut and thereafter sutured or stapled back together. As the digestive tract contains numerous enzymes, strong acids and multiple species of bacteria that assist with digestion, any perforation of a digestive organ is particularly problematic due to the likelihood of leakage therefrom and/or serious infection. As such, these procedures are typically difficult to perform correctly, have high rates of catastrophic post-operative complications that may require prolonged hospitalization and even additional operations, and are often irreversible and/or permanently affect the remodeled tissue and/or organ. Accordingly, a need exists for safe and effective devices and methods for remodeling organs and tissue that are reversible and do not require cutting the underlying tissue and/or the use of sutures or staples.

In addition to remodeling the digestive tract for the treatment of obesity, it is conventionally known to treat various other indications through providing support to the organ or tissue and/or organ or tissue remodeling. For example and without limitation, patients suffering from a symptomatic hiatal hernia may be treated by a Nissen fundoplication where the gastric fundus (the upper portion) of the stomach is wrapped, or plicated, around the inferior part of the esophagus and secured to itself through the use of sutures or staples. In this manner, the gastric fundus of the stomach blocks the enlarged hiatus in the diaphragm and prevents herniation of the stomach therethrough as well as the reflux of gastric acid. As with bariatric surgeries, a Nissen fundoplacation requires that the stomach wall is sutured in order to secure it in position around the esophagus, thereby increasing the risk of complications and preventing the procedure from being easily reversed.

Two laparoscopic surgical techniques exist as alternatives to a Nissen fundoplacation: Tension-Free Techniques and Non-Tension-Free Techniques (referring to the resulting tension—or lack thereof—of the lateral portions of the diaphragm after the procedure). In one example of a Tension-Free Technique, a triangular or semilunar polytef patch is positioned to occlude the anterior segment of the hiatus, which is fixed to the diaphragm with staples or stitches. In conjunction, the stomach is fixed to the abdomen and a fundoplication is performed. The same technique is used for the posterior segment of the hiatus. Conversely, in Non-Tension-Free Techniques, the most common method for hiatal closure is the use of simple stitches or a continuous suture to approach the crural of the diaphragm. Teflon® or Dacron® pledgets or a polypropylene strip are conventionally used to avoid the cutting stitches effect. The pillar closure is covered by a long strip of mesh, which is positioned below the diaphragm in order to reduce the risk of dysphagia or erosion by avoiding the encircling of the oesophagus.

Even when hiatal hernia surgical procedures are a success, the hiatal repair often subsequently fails due to tissue tension. The hiatal crus is a fleshy structure lacking tendinous reinforcement and the use of ordinary sutures to close the hiatal hernia runs a relatively high risk of cutting the muscle. If the hiatus is predominantly wide and the diaphragmatic pillars are necessarily approached with suturing as indicated in many of the above-described techniques, the lateral portions of the diaphragm close to the crura become tense, with probable risk of disruption. Furthermore, in addition to the specific indications discussed herein, there are numerous other conditions for the treatment of which organ and/or tissue remodeling procedures are conventionally employed.

Additionally, it is known to treat various other indications through providing support to an organ or tissue. Abdominal aortic aneurysm is one example of an indication for which conventional techniques of treatment are rather invasive and often require open surgery. An abdominal aortic aneurysm occurs when the large blood vessel that supplies blood to the abdomen, pelvis, and legs becomes abnormally large or balloons outward, thereby forming an aneurysm sac. If left untreated, this weakened area of the aortic wall can progress to aortic dissection or even rupture.

Conventionally, treatment for an abdominal aortic aneurysm involves either open aneurysm repair or endovascular stent grafting. Specifically, traditional open repair involves open abdominal surgery where the abnormal vessel is replaced with a graft made of synthetic material, such as Dacron®. Accordingly, the synthetic graft replaces the weakened area of the aorta and is sutured at its proximal and distal end to the remaining healthy aortic wall. In this manner, the graft allows blood to pass easily therethrough.

Endovascular abdominal aortic aneurysm repair (“EVAR”) is considered an accepted alternative to standard open surgery and avoids major intraabdominal (or retroperitoneal) surgery and the related morbidity and mortality that are associated with standard surgical repair. EVAR is an alternative procedure used in an effort to reinforce or strengthen the weakened aneurysmic area of the aorta that is performed laparoscopically. EVAR typically involves the advancement of a stent graft comprising fabric and metal mesh through the femoral artery and to the afflicted area. Placement of the graft is then achieved such that the graft is positioned within the weakened aortic location of the aneurysm. In this procedure, the proximal and distal ends of the endovascular graft are sutured to healthy portions of the aorta, both proximal and distal to the aortic aneurysm region. Accordingly, the bulge of the aneurysm sac remains; however, the endovascular graft ideally allows blood to flow through the graft and thus bypassing the aneurysm sac.

While EVAR is less invasive than open aneurysm repair, the EVAR procedure typically requires lifelong surveillance by imaging after endograft placement to ensure that the graft continues to function properly. The most common complication associated with EVAR is endoleak. Endoleaks are defined as areas of persistent blood flow outside the lumen of the endograft, either within the aneurysm sac or within connected vascular segments bypassed by the graft. An endoleak following EVAR is considered a failure of the procedure as it is associated with aneurysm enlargement or even rupture. Presence of an endoleak may require additional endovascular interventions or conversion to open repair. Other complications commonly associated with conventional aneurysmic repair procedures include graft migration, thrombosis and/or kinking of the graft. Accordingly, a need exists for safe and effective devices and methods for providing support to weakened or damaged tissue that are noninvasive and reduce or altogether prevent the complications commonly associated with conventionally known support procedures.

It will be appreciated that the foregoing examples were only provided as examples and that there are numerous other indications where intervention is necessary either to remodel the underlying organ or tissue and/or to provide support thereto.

### SUMMARY

Embodiments of devices and methods are described for delivering an instrument laparoscopically to a targeted tissue. In at least one embodiment a delivery device is described, the delivery device comprising a first arm, a second arm, a handle, and a lift system. The first arm and the second arm are both capable of slidable movement relative to each other. The handle comprises a first activation system, which is coupled with the first arm and the second arm and configured to slidably move the first arm and the second arm relative to each other. The lift system is coupled with the first and second arms and comprises a first mounting bracket and a second mounting bracket. Additionally, the lift system is configured to move the first and second mounting brackets between a substantially closed position and a substantially open position upon manipulation of the first activation system of the handle. Furthermore, the delivery device may be configured such that manipulation of the first activation system of the handle moves the first and second mounting brackets between the substantially closed position and the substantially open position in a parallel fashion. In at least one embodiment, the first activation system comprises a lever.

The delivery device may be capable of laparoscopic insertion into and advancement through a body. In at least one embodiment, the delivery device may further comprise a hollow casing comprising a first end, a second end and interior extending between the first and second ends, the interior of the hollow casing comprising the first and second arms slidably disposed therein.

As previously described, the lift system of the delivery device comprises a first and second mounting bracket. The first mounting bracket may comprise a first coupling mechanism configured to releasably couple with the first component of an instrument. Likewise, the second mounting bracket of the lift system may comprise a second coupling mechanism configured to releasably couple with a second component of the instrument. Further, the handle of the delivery device may comprise a second activation system coupled with the first and second coupling mechanisms. The second activation system may comprise a lever. In at least one embodiment, the second activation system is configured to release the first and second components of the instrument from the first and second mounting brackets of the lift system upon manipulation thereof.

In certain embodiments, the second activation system comprises a first cable extending between the second activation system and the first mounting bracket and a second cable extending between the second activation system and the second mounting bracket. In addition, the first coupling mechanism may comprise one or more posts configured to releasably couple with the first component of the instrument. Furthermore, the second coupling mechanism may comprise one or more posts configured to releasably couple with the second component of the instrument. In at least one embodiment, the first cable of the second activation system is coupled with each of the one or more posts of the first coupling mechanism and the second cable of the second activation system is coupled with each of the one or more posts of the second coupling mechanism.

Each of the first and second mounting brackets of the lift system may further comprise at least one slidable component. Further, each of the one or more posts of the first coupling mechanism may be mounted on one of the at least one slidable components of the first mounting bracket and each of the one or more posts of the second coupling mechanism may be mounted on one of the at least one slidable components of the second mounting bracket.

Referring back to the instrument that may be coupled with the first and second mounting brackets of the lift system, the first component of the instrument may further comprise one or more pins moveable between a substantially retracted position and a substantially extended position, and the handle of the delivery device further comprises a third activation system. In this at least one embodiment, the third activation system of the delivery device is coupled with the first coupling mechanism of the lift system and is configured to move the one or more pins of the first component of the instrument from the substantially retracted position to the substantially extended position. The third activation system may comprise a lever.

The third activation system may further comprise at least one cable extending between the third activation system and the first mounting bracket. Furthermore, the first coupling mechanism may further comprise a latch mechanism configured to releasably couple with the first component of the instrument such that the latch mechanism is capable of interacting with and affecting the position of the one or more pins of the instrument. In at least one embodiment, the first mounting bracket may further comprise one or more slidable components and the latch mechanism may be mounted on one of the at least one slidable components of the first mounting bracket. The at least one cable of the third activation system may be coupled with the latch mechanism of the first coupling mechanism.

As described herein, the handle of the delivery device may comprise the first activation system, the second activation system and the third activation system, wherein the first activation system is capable of moving the first and second arms of the delivery device relative to one another, thereby opening and closing the lift system; the second activation system is configured to release the first and second components of the instrument from the first and second mounting brackets; and the third activation system is configured to move the one or more pins of the first component of the instrument from the substantially retracted position to the substantially extended position. Both the second and third activation systems may each comprise a lever, both of which are hingedly coupled with a pivot point. Further, in at least one embodiment, the second and third activation systems may be independently moveable relative to each other around the same pivot point.

Additional embodiments of a delivery device for facilitating the laparoscopic delivery of an instrument to a targeted tissue are described herein. Such embodiments comprise a hollow casing, a first arm and a second arm both of which are slidably disposed within the hollow casing, a lift system and a handle. The hollow casing of the delivery device may comprise an elongated tube having a proximal end and a distal end connected by a body having a hollow interior, the distal end of the hollow casing capable of laparoscopic introduction into a body. The first arm may comprise a proximal end and a distal end and the second arm may comprise a proximal end and a distal end. The lift system may comprise a first set of shafts, a second set of shafts, a first mounting bracket and a second mounting bracket, the first set of shafts coupled with the distal end of the first arm and the first mounting bracket and the second set of shafts coupled with the distal end of the second arm and the second mounting bracket such that the first and second mounting brackets are pushed away or pulled toward each other in a parallel fashion in connection with the slidable movement of the first and second arms. The handle is coupled with the proximal end of the first arm, the proximal end of the second arm and the proximal end of the hollow casing. Furthermore, the handle may comprise a first activation system capable of slidably moving the first and second arms relative to each other. In at least one embodiment, the first activation system comprises at least one lever coupled with the proximal end of the first arm and the proximal end of the second arm.

In the previously described delivery device, the first and second mounting brackets may each be configured to releasably couple with one or more components of an instrument. Furthermore, the handle may further comprise a second activation system in communication with the first and second mounting brackets of the lift system. The second activation system may comprise a first element configured to uncouple the one or more components of the instrument from the first and second mounting brackets of the lift system upon manipulation thereof. Additionally, the second activation system may comprise a second element configured to communicate with one or more pins of the one or more components of the instrument and move the one or more pins from a substantially retracted position to a substantially extended position upon manipulation thereof. Both the first and second activation systems of the delivery device may be hingedly moveable around a pivot point.

Methods for delivering an instrument to a tissue of interest are additionally described herein. In at least one embodiment, a method for delivering an instrument to a tissue of interest comprises the steps of providing a delivery device for facilitating the laparoscopic delivery of the instrument to a targeted tissue, the delivery device comprising: a first arm and a second arm, both the first and second arms capable of slidable movement relative to each other, a handle comprising a first activation system, the first activation system coupled with the first arm and the second arm and configured to slidably move the first arm and the second arm relative to each other, and a lift system coupled with the first and second arms, the lift system comprising a first mounting bracket and a second mounting bracket and configured to move the first mounting bracket and the second mounting bracket between a substantially closed position and a substantially open position upon manipulation of the first activation system of the handle, wherein the first mounting bracket is releasably coupled with a first component of an instrument and the second mounting bracket is releasably coupled with a second component of the instrument; positioning the first component of the instrument adjacent to a first surface of a targeted tissue; positioning the second component of the instrument adjacent to a second surface of a targeted tissue; and closing the first and second components of the instrument around the targeted tissue in a parallel fashion through operation of the first activation system of the delivery device. Further, the above-described method may include the step of moving the first and second mounting brackets to a substantially open position through operation of the first activation system. The method may also comprise the additional step of withdrawing the delivery device from the targeted tissue, thereby allowing the instrument to remain thereon.

In at least one embodiment, the steps of positioning the first component of the instrument adjacent to a first surface of a targeted tissue and positioning the second component of the instrument adjacent to a second surface of a targeted tissue occur simultaneously. Furthermore, the handle of the delivery device may further comprise a second activation system coupled with the first and second mounting brackets, the second activation system configured to release the first and second components of the instrument from the first and second mounting brackets upon manipulation thereof. In at least one embodiment the method, the method may further comprise the step of releasing the first and second components of the instrument from the delivery device through operation of the second activation system of the handle.

The second activation system of the delivery device provided by the method may additionally comprise a first cable extending between the second activation system and the first mounting bracket and a second cable extending between the second activation system and the second mounting bracket. In this at least one embodiment, the step of releasing the first and second components of the instrument from the delivery device through operation of the second activation system of the handle may further comprise pulling the first and second cables through operation of the second activation system.

The first mounting bracket of the delivery device provided by the method may additionally comprise one or more posts configured to releasably couple with the first component of the instrument and the second mounting bracket further comprises one or more posts configured to releasably couple with the second component of the instrument. In this at least one embodiment the step of releasing the first and second components of the instrument from the delivery device through operation of the second activation system of the handle may further comprises withdrawing the one or more posts of the first and second mounting brackets from the first and second components of the instrument, respectively, through operation of the second activation system.

The first component of the instrument may additionally comprise one or more pins moveable between a substantially retracted position and a substantially extended position and the second activation system of the delivery device may be configured to move the one or more pins of the first component of the instrument from the substantially retracted position to the substantially extended position when the first component of the instrument is releasably coupled when the first mounting bracket. In this at least one embodiment, the method for laparoscopically delivering a device to a targeted tissue may further comprise the step of moving the one or more pins of the first component of the instrument into the substantially extended position through operation of the second activation mechanism.

The device described herein is further described as being included within a kit for performing a medical procedure. In at least one embodiment, the kit may comprise an instrument comprising one or more components; a fluoroscope; and a delivery device comprising a first arm and a second arm, both the first and second arms capable of slidable movement relative to each other, a handle comprising a first activation system, the first activation system coupled with the first arm and the second arm and configured to slidably move the first arm and the second arm relative to each other, and a lift system coupled with the first and second arms, the lift system comprising a first mounting bracket and a second mounting bracket and configured to move the first mounting bracket and the second mounting bracket between a substantially closed position and a substantially open position in a parallel fashion upon manipulation of the first activation system of the handle.

### BRIEF DESCRIPTION OF THE DRAWINGS

FIG. 1A shows a perspective view of at least one embodiment of a remodeling device for remodeling and/or supporting a tissue or organ.

FIG. 1B shows a top view a component of at least one embodiment of the remodeling device shown in FIG. 1A.

FIG. 1C shows a cross-sectional view of the component of the at least one embodiment of the remodeling device shown in FIG. 1B, taken along line A-A of FIG. 1A.

FIG. 1D shows a side view of the components of the remodeling device of FIG. 1A mechanically engaged with one another.

FIG. 2 shows a top view of at least one alternative configuration of the remodeling device shown in FIG. 1A.

FIG. 3 shows a side view of at least one alternative configuration the remodeling device shown in FIG. 1A.

FIGS. 4A and 4B show perspective views of a component of the remodeling device of FIG. 1A having moveable pins extending therefrom.

FIG. 5A shows a schematic view of at least one embodiment of the remodeling device of FIGS. 1A-1D.

FIG. 5B shows the remodeling device of FIG. 5A applied to a targeted tissue.

FIG. 5C shows a cross-sectional view of at least one embodiment of the remodeling device of FIG. 5A further comprising a resistance mechanism.

FIG. 6A shows a cross-sectional view of at least one pin of the remodeling device of FIGS. 5A and 5B.

FIGS. 6B and 6C show embodiments of a pin of the remodeling device of FIGS. 5A and 5B.

FIGS. 7A and 7B show perspective views of at least one embodiment of the remodeling device of FIGS. 5A and 5B applied to an underlying targeted tissue.

FIG. 8A shows a side view of one component of the remodeling device of FIG. 1A, as well as enlarged views of at least one embodiment of the pins of the remodeling device of FIG. 1A.

FIG. 8B shows a cross sectional view of a pin of FIG. 8A mechanically engaged with a second component of the remodeling device of FIG. 1A.

FIG. 9 shows a top view of a component of at least one embodiment of the remodeling device shown in FIG. 1A being released from another component of the at least one embodiment of the remodeling device of FIG. 1A.

FIG. 10A shows a perspective view of at least one embodiment of a remodeling device for remodeling and/or supporting a tissue or organ, the remodeling device comprising at least one embodiment of an engagement mechanism.

FIG. 10B shows an enlarged view of the engagement mechanism of the remodeling device of FIG. 10A.

FIG. 11A shows a perspective view of at least one embodiment of a remodeling device for remodeling and/or supporting a tissue or organ, the remodeling device comprising an alternative embodiment of an engagement mechanism.

FIGS. 11B and 11C show enlarged views of the engagement mechanism of the remodeling device of FIG. 11A.

FIG. 12A shows a perspective view of at least one embodiment of a remodeling device for remodeling and/or supporting a tissue or organ, the remodeling device comprising an alternative embodiment of an engagement mechanism.

FIG. 12B shows an enlarged view of the engagement mechanism of the remodeling device of FIG. 12A.

FIG. 12C shows a cross-sectional view of the engagement mechanism of FIG. 12A.

FIG. 13A shows a side view of the remodeling device of FIG. 1A positioned on a stomach.

FIG. 13B shows a cross-sectional view of the stomach of FIG. 13A taken along line B-B.

FIGS. 14A and 14B show perspective views of at least one embodiment of a remodeling device for remodeling and/or supporting a tissue or organ.

FIG. 15A shows a side view of the remodeling device of FIGS. 14A and 14B positioned on a stomach.

FIG. 15B shows a cross-sectional view of the stomach of FIG. 15A taken along line C-C.

FIG. 16A shows a perspective view of at least one embodiment of a remodeling device for remodeling and/or supporting a tissue or organ.

FIGS. 16B and 16C show side views of the remodeling device of FIG. 16A in open and closed configurations.

FIG. 17 shows a side view of the remodeling device of FIGS. 16A-16C positioned on a stomach.

FIGS. 18A-18C show perspective views of a delivery device for delivering the remodeling devices disclosed herein to a targeted tissue.

FIG. 19 shows a cross-sectional view of the handle of the delivery device of FIGS. 18A-18C.

FIG. 20 shows a perspective view of the handle of FIG. 19.

FIGS. 21A-21C show perspective views of the lift system of the delivery device shown in FIGS. 18A-18C.

FIGS. 22A and 22B show perspective views of a first mechanical coupling of the delivery device of FIGS. 18A-18C.

FIG. 22C shows a top view of a component of at least one embodiment of a remodeling device for remodeling and/or supporting a tissue or organ, the remodeling device configured to releasably couple with the mechanical coupling of FIGS. 22A and 22B.

FIG. 22D shows a schematic view of the proximal end of the component of the remodeling device of FIG. 22C.

FIGS. 23A and 23B show cross-sectional views of the pin portions of the component of the remodeling device of FIG. 22C.

FIGS. 24A and 24B show perspective views of a second mechanical coupling of the delivery device of FIGS. 18A-18C.

FIG. 24C shows a schematic view of the proximal end of a component of at least one embodiment of a remodeling device for remodeling and/or supporting a tissue or organ, the remodeling device configured to releasably couple with the mechanical coupling of FIGS. 24A and 24B.

FIG. 25 shows a flow chart of a method for laparoscopically delivering embodiments of the remodeling device disclosed herein to a targeted tissue.

FIG. 26 shows a flow chart of a method for laparoscopically delivering embodiments of the remodeling device disclosed herein through the use of the delivery device of FIGS. 18A-18C.

FIGS. 27A-27F show perspective illustrations of the steps of the method shown in FIG. 26.

FIGS. 28A-28C show perspective views of a clamp device for delivering a stacking clamp to the remodeling devices disclosed herein in order to facilitate secure placement of the same on a targeted tissue.

FIG. 29 shows a flow chart of a method for laparoscopically delivering embodiments of the remodeling device disclosed herein through the use of the delivery device of FIGS. 18A-18C and the clamp device of FIGS. 28A-28C.

FIG. 30 shows a perspective view of the delivery device of FIGS. 18A-18C coupled with the remodeling device disclosed herein, both in conjunction with the clamp device of FIGS. 28A-28C.

### DETAILED DESCRIPTION

Reference will now be made to the embodiments illustrated in the drawings and specific language will be used to describe the same. It will nevertheless be understood that no limitation of scope is intended by the description of these embodiments.

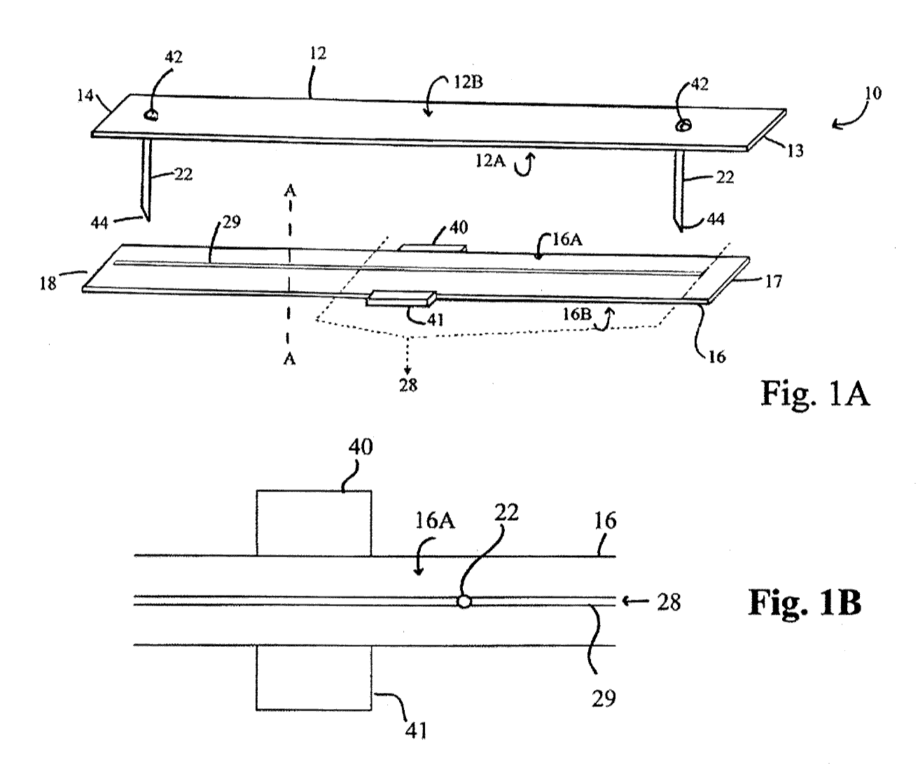

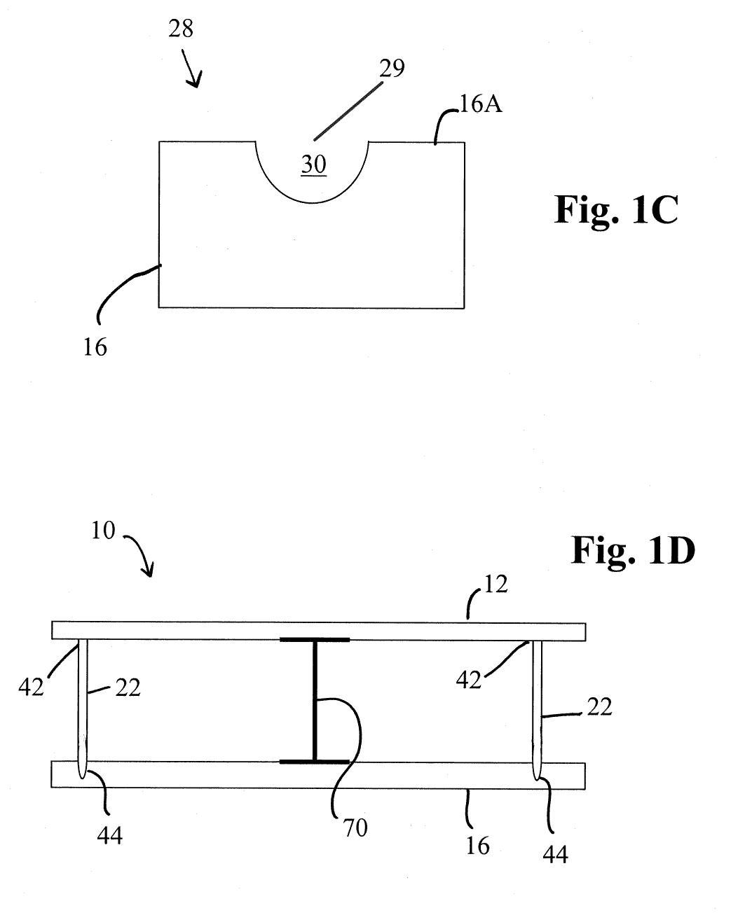

FIGS. 1A, 1B and 1D show schematic views of a remodeling device 10 for remodeling a tissue or organ. In addition, FIG. 1C shows a cross-sectional view of a component of the remodeling device 10 taken along line A-A of FIG. 1A. In the at least one embodiment of the remodeling device 10 shown in FIGS. 1A-1D, the remodeling device 10 comprises an implantable device and does not require sutures or staples that could lead to dehiscence (e.g., an abnormal connection between organs or tissue), or other complications. In addition, while the remodeling device 10 is available for temporary and chronic placement within a patient's body, remodeling procedures performed through the use of the device 10 are reversible through minimally invasive procedures.

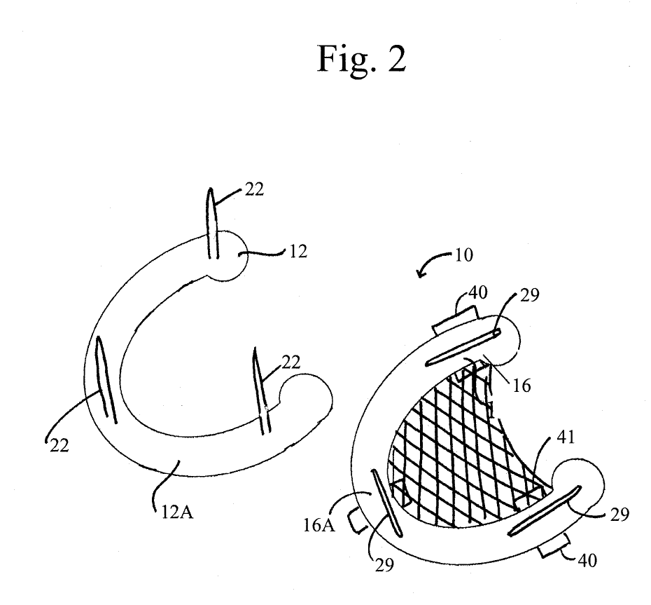

Now referring to FIG. 1A, the remodeling device 10 is comprised of a first component 12 and a second component 16. The first component 12 comprises a first shape and the second component 16 comprises a second shape that matches at least a portion of the first shape of the first component 12. For example, and without limitation, the first and second components 12, 16 may be configured in a straight bar configuration as shown in FIG. 1A. Alternatively, the first and second components 12, 16 may be configured in a curved, circular or other configuration. In addition, the first and second components 12, 16 may be configured such that the shape of each of the components 12, 16 defines an interior 70 as shown in FIG. 2. In at least one embodiment, the first and/or second component 12, 16 may comprise a section of mesh disposed across a portion of such interior as described in detail in U.S. patent application Ser. No. 12/307,113, filed Dec. 30, 2008 and International Application Number PCT/U.S.07/15267, filed Jun. 29, 2007, which are both incorporated by reference herein. It will be understood that the first and second components 12, 16 of the remodeling device 10 may be configured in any shape and may be flexible, semi-flexible, or articulated. Furthermore, it is contemplated that a clinician may select the desired configuration of the components 12, 16 of the remodeling device 10 based on the particular patient that is being treated and/or pursuant to the application for which the remodeling device 10 is being used to ensure that the remodeling device 10 appropriately conforms to the tissue or organ of interest.

The first component 12 of the remodeling device 10 comprises a proximal end 13, a body having a first side 12A and a second side 12B, and a distal end 14; The first side 12A of the first component 12 is configured to be positioned adjacent to or in contact with a tissue or organ of interest. Likewise, the second component 16 comprises a proximal end 17, a body having a first side 16A and a second side 16B, and a distal end 18. The first side 16A of the second component 16 is configured to be positioned adjacent to or in contact with the tissue or organ of interest.

The first component 12 and the second component 16 each comprise a material suitable to resist corrosion, such as and without limitation, polyurethane, polytetrafluoroethylene (“PTFE”), silastic, titanium, stainless steel or any other material suitable for use in the medical arts that is corrosion resistant. Accordingly, the remodeling device 10 can withstand chronic placement within a body without the risk of deterioration. In at least one embodiment, the first and second components 12, 16 of the remodeling device 10 are comprised of ultra high density polyethylene.

The remodeling device 10 further comprises at least one pin 22. Each of the at least one pins 22 comprises a rigid material and may comprise any diameter that is suitable for the particular application. In at least one embodiment, the pins 22 are comprised of a material suitable to resist corrosion, such as and without limitation, polyurethane, PTFE, silastic, titanium, stainless steel or any other material suitable for use in the medical arts that is corrosion resistant. Further, in at least one embodiment, each of the at least one pins 22 of the remodeling device 10 comprises a diameter of less than or about 1 millimeter.

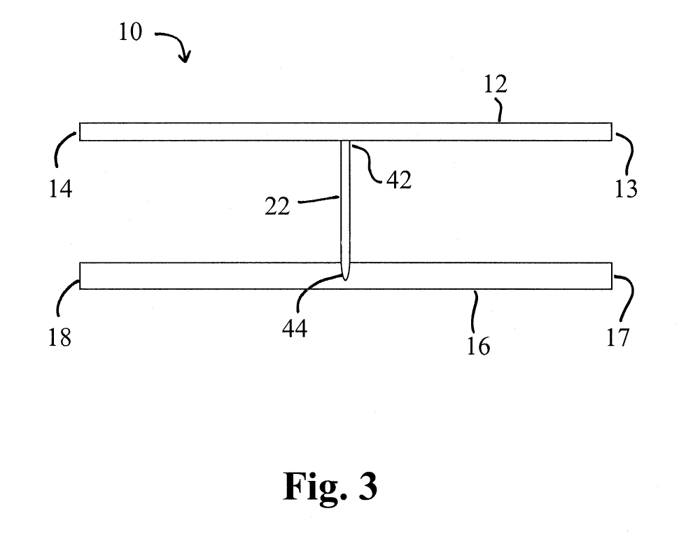

Referring back to FIG. 1A, in at least one embodiment, each of the at least one pins 22 may extend from the first side 12A of the first component 12 such that each pin 22 can mechanically engage the second component 16 when the first and second components 12, 16 are in an engaged configuration. It will be appreciated that the at least one pin 22 may be coupled with the first side 12A of the first component 12 in any location and any number of pins 22 may be used in connection with the remodeling device 10. For example, as shown in FIG. 3, the remodeling device 10 may only comprise a single pin 22. In at least one alternative embodiment of the remodeling device 10, at least one pin 22 extends from the first side 12A of the first component 12 and at least one pin 22 extends from the first side 16A of the second component. In this manner, each of the at least one pins 22 can mechanically engage the opposite component 12, 16 when the first and second components 12, 16 are in an engaged configuration. It is contemplated that the number of pins 22 of the remodeling device 10 and placement thereof with respect to the components of the remodeling device 10 will be determined based on the particular patient into which the remodeling device 10 is to be implanted, the tissue of interest, and the application for which the remodeling device 10 is to be used.

Each of the at least one of pins 22 comprises a proximal end 42 and a distal end 44. The proximal end 42 and the distal end 44 of each pin 22 may be configured similarly or differently, in a blunt, tapered or other type of configuration. In at least one embodiment, each of the at least one pins 22 is metallic and comprises a distal end 44 having a tapered configuration such that the pins 22 can easily puncture a tissue when the distal ends 44 are applied thereto.

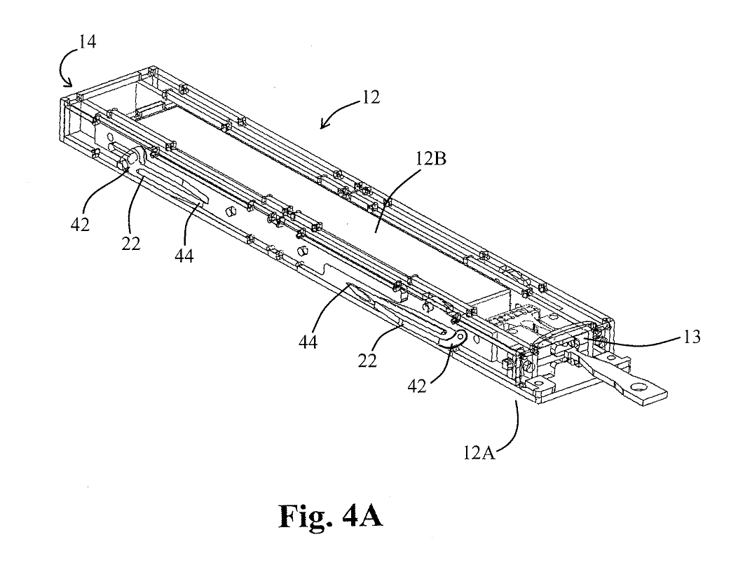

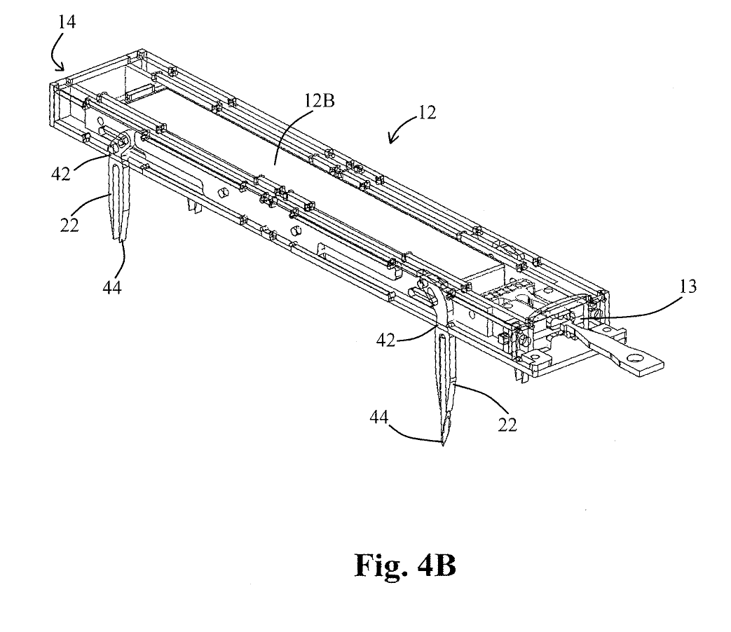

In at least one embodiment of the remodeling device 10, each of the pins 22 of the remodeling device 10 is capable of moving between a substantially retracted position wherein the pin 22 is positioned substantially parallel to the body of the first component 12 (see FIG. 4A), and a substantially extended position wherein the pin 22 is positioned substantially perpendicular to the body of the first component 12 (see FIG. 4B). In the at least one embodiment shown in FIGS. 4A and 4B, the proximal end 42 of each of the at least one pins 22 may be hingedly coupled with the first component 12. Further, where a remodeling device 10 comprises two or more pins 22, each of the pins 22 can move independently of the other pins 22 such that one or more of the pins 22 may be in the substantially retracted position while one or more of the pins 22 are in the substantially extended position.

The mobility of the at least one pin 22 between a substantially retracted and substantially extended position facilitates the laparoscopic delivery of the remodeling device 10 to the tissue of interest. For example, the device 10 may be advanced through the body with the at least one pin 22 in the retracted position in order to prevent inadvertent damage to surrounding tissue. In addition, introduction of the device 10 with the at least one pin 22 in the retracted position allows for the use of a laparoscopic port having a smaller diameter. After the components 12, 16 are positioned adjacent to the targeted tissue, the clinician can then deploy to the at least one pin 22 into the substantially extended position.

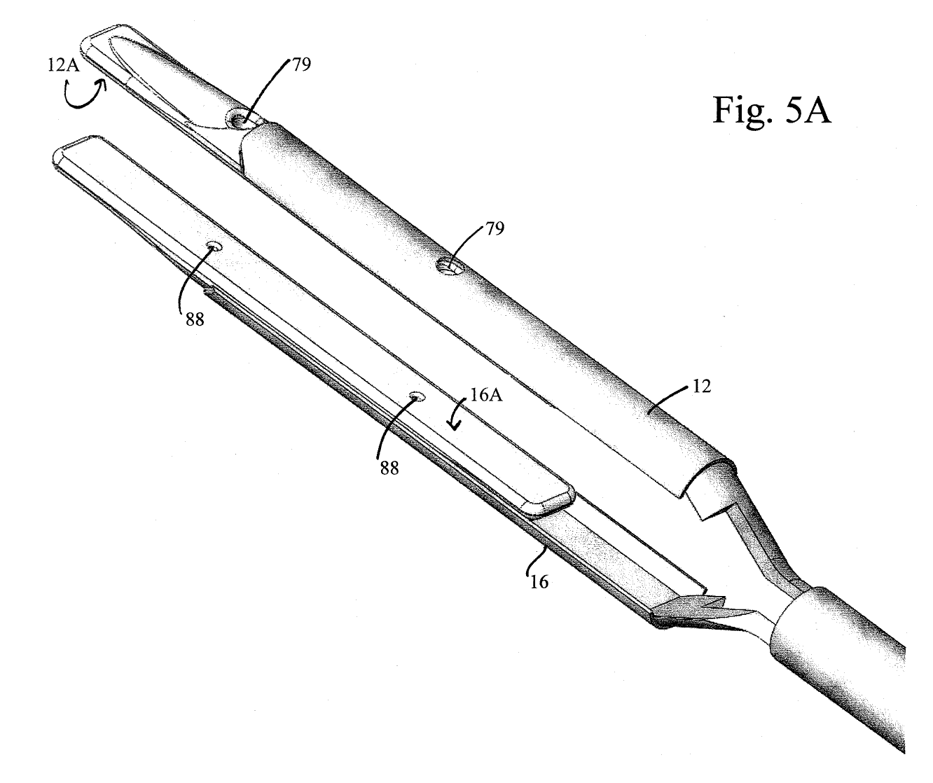

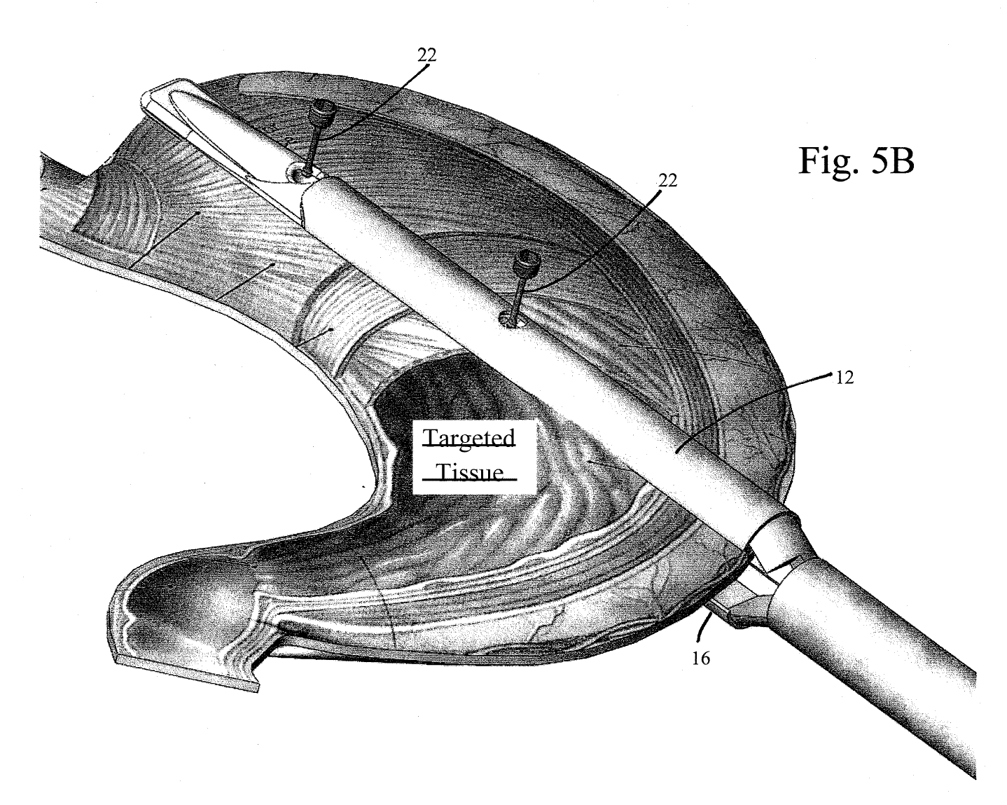

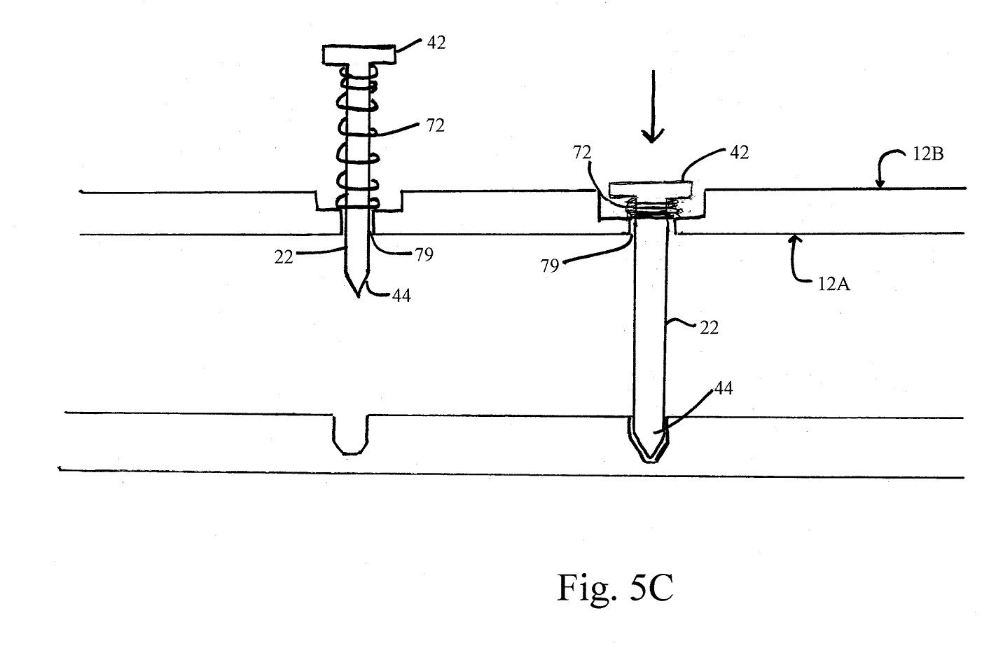

In an alternative embodiment, the at least one pin 22 of the remodeling device 1Q may initially be independent of either of the first or second components 12, 16. In this manner, the at least one pin 22 may resemble more of a conventional screw, wherein its proximal end 42 is larger in diameter than its body and distal end 44. As shown in FIG. 5A, in this at least one embodiment, the first component 12 comprises one or more holes 79 that are in communication with both the first and second sides 12A, 12B of the first component 12. Accordingly, the holes 79 are configured such that the body and distal end 44 of the pin 22 may be slidably received therethrough, resulting in the distal end 44 of the pin 22 extending from the first side 12A of the first component 12 (as shown in FIG. 1A). However, in this at least one embodiment, the diameter of each of the at least one holes 79 is less than the diameter of the proximal end 42 of each of the at least one pins 22. In this manner, when a pin 22 is inserted through a hole 79 in the first component 12, the proximal end 42 of the pin 22 is unable to fit through the hole 79 and is thus retained therein. As shown in FIG. 5B, in application, after the components 12, 16 of the remodeling device 10 are positioned adjacent to the targeted tissue, a clinician can insert the at least one pin 22 into the body laparoscopically and thread the same through the appropriate holes 79 in the first component 12 using a laparoscopic device inserted through a separate abdominal port.

In at least one embodiment, a resistance mechanism may further be coupled with each of the pins 22 to provide bias such that each of the pins 22 is prevented from fully extending from the first side 12A of the first component 12 until a sufficient downward force is applied to the proximal end 42 of each pin 12. In the non-limiting example shown in FIG. 5C, the resistance mechanism comprises a spring 72 coiled around each of the pins 22 such that when the pin 22 is inserted into a hole 79 in the first component 12, the spring 72 is sandwiched in between the proximal end 42 of the pin and the hole 79.

In this at least one embodiment, the spring 72 is biased such that when no downward force is applied to the proximal end 42 of the pin 22 and/or the distal end 44 of the pin 22 is not locked in place within the second component 16, the spring 72 remains in an expanded position and stores little, if any, potential energy. However, when downward force is applied to the proximal end 42 of the pin 22 (e.g., to drive the distal end 44 of the pin 22 through a targeted tissue) the spring 72 is compressed and thus stores potential energy. In this manner, the spring 72 of the resistance mechanism can provide enough resistance that each pin 22 having the spring 72 coupled therewith is prevented from moving into a fully extended position until pressure is applied.

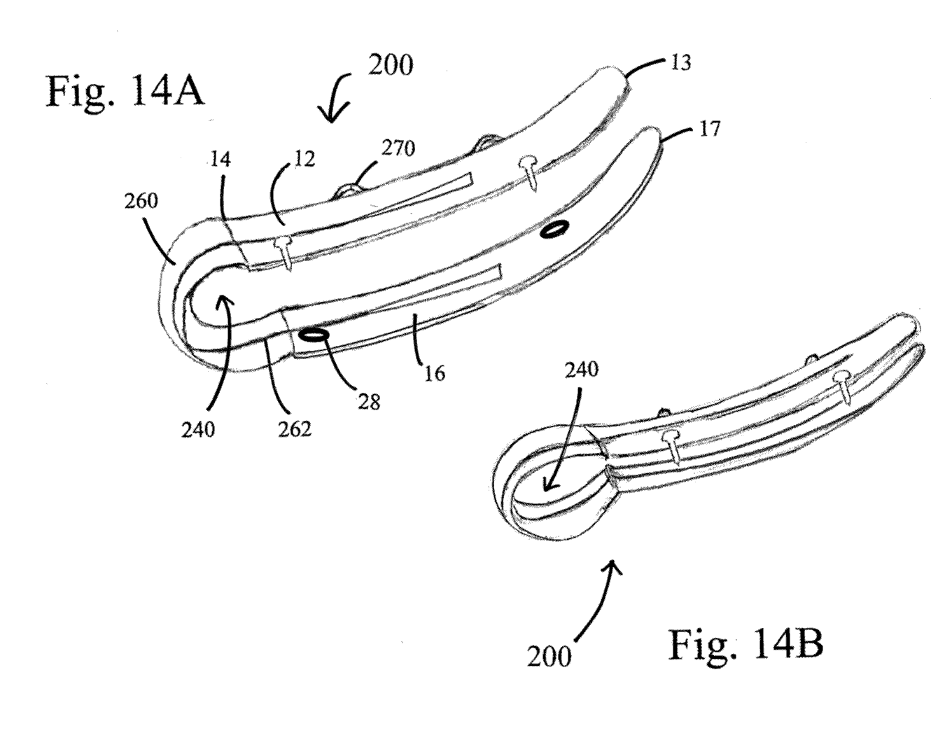

Inclusion of the resistance mechanism with the pins 22 of the remodeling device 10 facilitates the safe laparoscopic delivery of the remodeling component 10 to the targeted tissue by decreasing the likelihood that the distal ends 44 of such pins 22 will inadvertently interact with a tissue upon delivery of the device 10. This is especially applicable with respect to certain embodiments of the remodeling device 10 described herein where the first and second components 12, 16 are coupled together prior to delivery of the device 10 (see FIGS. 14A-17). While embodiments of the spring 72 have been described herein as an example of the resistance mechanism, it will be appreciated that the resistance mechanism may comprise any other device capable of providing resistance against the one or more pins 22 fully extending through the holes 79 of the first component 12.

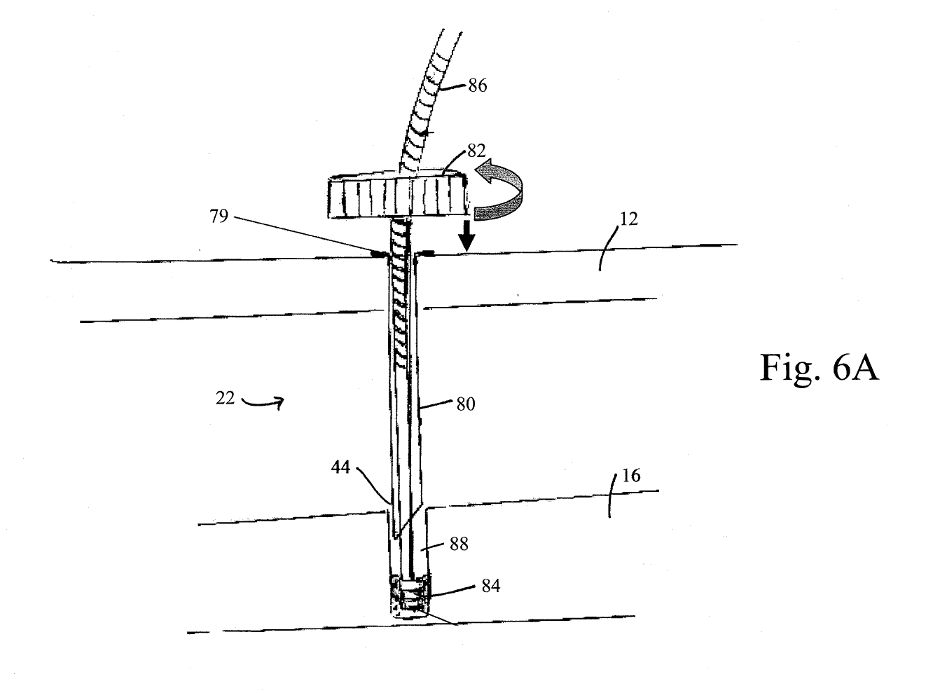

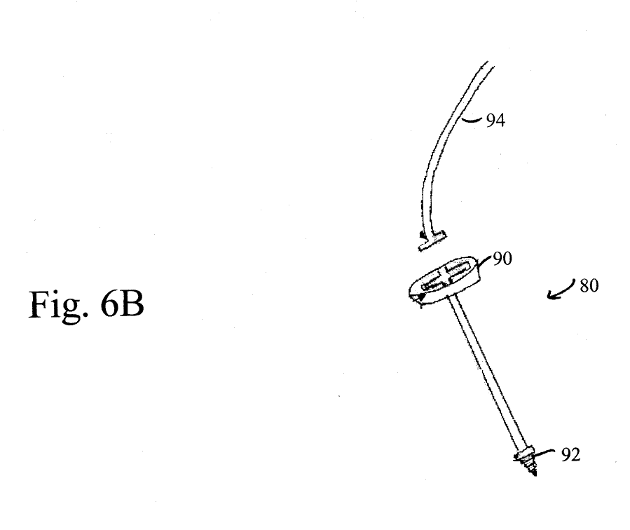

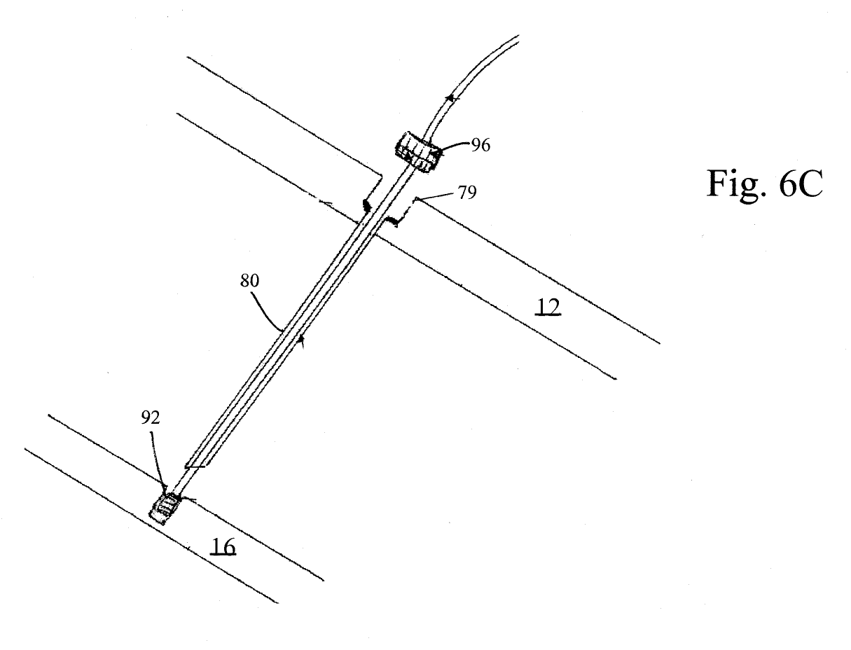

Now referring to FIG. 6A, at least one alternative embodiment of the remodeling device 10 is shown. In this at least one alternative embodiment, the remodeling device 10 may comprise at least one independent pin 22, however, the at least one pin 22 comprises a needle pin 80 having an open proximal end 42, an open distal end 44 and a body having hollow interior extending therebetween. The proximal end 42 of the needle pin 80 further comprises a screw nut 82 comprising a diameter that is larger than the one or more holes 79 disposed within the first component 12. The screw nut 82 of the needle pin 80 may comprise a magnetic material and have an opening therein, the opening in the screw nut 82 in communication with the hollow interior of the needle pin 80 and comprising screw-like threading. The distal end 44 and the body of the needle pin 80 are configured to be slidably inserted through the one or more holes 79 disposed in the first component 12.

When the distal end 44 of the needle pin 80 is inserted through the one or more holes 79 in the first component 12, because the screw nut 82 of the proximal end 42 of the needle pin 80 has a diameter that is larger than the holes 79, the needle pin 80 is prevented from fully traversing the hole 79 in which it is inserted. Accordingly, as shown in FIG. 6A, when the needle pin 80 is completely inserted into a hole 79 of the first component 12, the screw nut 82 retains the needle pin 80 such that the distal end 44 of the needle pin 80 is positioned adjacent to the first side 16A of the second component 16. Furthermore, in at least one embodiment, the first side 16A of the second component 16 may comprise one or more indentations 88 disposed therein. In this embodiment, the body of the needle pin 80 may optionally be longer such that the distal end 44 may at least partially extend within the relevant indentation 88.

In this at least one embodiment, the at least one needle pin 80 is used in conjunction with a screw bar 86 having a screw 84 positioned at its distal-most end. The screw bar 86 may be configured for laparoscopic insertion with a body and may be flexible, semi-flexible or rigid. However, the screw bar 86 comprises sufficient torsional rigidity such that it can be used to rotate the screw 84 positioned at its distal end. The screw 84 of the screw bar 86 may be comprised of any material and, in at least one embodiment, comprises a ferromagnetic material. In addition, the screw 84 of the screw bar 86 comprises screw-like threads extending around the exterior thereof. Likewise, a portion of the body of the screw bar 86 may also comprise a plurality of screw-like threads extending along the exterior thereof.

The hollow interior of the needle pin 80 is configured to receive the distal end (i.e. the screw 84) of the screw bar 86 therethrough. In this manner, the screw 84 and the screw bar 86 are configured such that they can be easily inserted into the opening of the screw nut 82 and advanced through the interior and the open distal end 44 of the needle pin 80. It will be appreciated that when the screw bar 86 is inserted into the opening of the screw nut 82, the threads of the screw 84 and the threads of the body of the screw bar 86 are configured to engage with the threads within the opening of the screw nut 82. Accordingly, the screw bar 86 is advanced through the interior of the needle pin 80 by rotating the threads of the screw 84 and/or the screw bar 86 therethrough. Furthermore, the screw nut 82 may be calibrated such that a one full rotation of the screw nut 82 is associated with a defined linear distance covered by the screw bar 86. For example and without limitation, the screw nut 82 may be calibrated to 1 millimeter per full rotation such that the screw bar 86 advances therethrough 1 millimeter per full turn of the screw nut 82.

As is illustrated in FIG. 6A, the screw bar 86 may extend the entire length of the needle pin 80. Due to the placement of the distal end 44 of the needle pin 80 relative to the first side 16A of the second component 16 when the hollow needle pin 80 is inserted through the at least one hole of the first component 12, when the screw bar 86 is inserted through the interior of the needle pin 80 and advanced through the open distal end 44 thereof, the screw 84 of the screw bar 86 is capable of interacting with the first side 16A of the second component 16. Specifically, in the at least one embodiment wherein the first side 16A of the first component 16 further comprises indentations 88, the screw 84 is configured to be inserted into an indentation 88 such that the screw-like threads of the exterior of the screw 84 engage with the sides of the indentation. In this manner, the screw 84 is capable of being securely seated within the first side 16A of the second component 16. Furthermore, due to the calibration of the screw nut 82, a clinician can identify exactly how far apart the first and second bars 12, 16 of the remodeling device 10 are disposed based on the number of rotations made by the screw nut 82.

Now referring to FIG. 6B, an alternative embodiment of the needle pin 80 configuration is shown. In this at least one embodiment, while the needle pin 80 still comprises a proximal end 42, a distal end 44, and a body extending therebetween, both the proximal and distal ends 42, 44 of the needle pin 80 need not be open. Rather, in this at least one embodiment, the proximal end 42 of the needle pin 80 comprises a screw top 90 and the distal end 44 of the needle pin 80 comprises a screw 92. In the at least one embodiment shown in FIG. 6B, the screw 92 of the distal end 44 of the needle pin 80 comprises a conical configuration; however, it will be appreciated that it may comprise any configuration so long as the screw 92 is capable of threaded engagement with an indentation 88 disposed in the first side 16A of the second component 16. For example and without limitation, as shown in FIG. 6C, the screw 92 may alternatively comprise a cylindrical configuration.

Similar to the prior embodiments described with respect to the needle pin 80, the screw top 90 of the proximal end 42 of the needle pin 80 has a diameter that is larger than the holes 79 such that the needle pin 80 is prevented from fully traversing the hole 79 in which it is inserted. The screw top 90 may further be configured such that its proximal-most portion comprises a receptacle configured to receive a magnetic twister or torque catheter 94 therein (see FIG. 6B). Additionally, the screw top 90 may be comprised of a ferromagnetic material to facilitate interaction between the screw top 90 and the twister or torque catheter 94. Accordingly, when the twister or torque catheter 94 is engaged with the screw top 90, a clinician can cause the screw 92 of the needle pin 80 to be more deeply seated within the indentation 88 of the second component 16 by rotating the needle pin 80 by way of the twister or torque catheter 94.

In yet another at alternative embodiment of the needle pin 80, the screw top 90 may be flat, but comprised of a ferromagnetic material and configured to be received within a magnetic folder 96 comprising an attractive polarity therewith (see FIG. 6C). Here, when the magnetic folder 96 is inserted into the body cavity laparoscopically, a clinician can insert the magnetic folder 96 over the screw top 90 and cause the screw 92 of the distal end 44 of the needle pin 80 to be more deeply seated within the indentation 88 of the second component through rotation of the needle pin 80. Specifically, in this at least one embodiment, when the magnetic folder 96 is inserted over the screw top 90, rotation of the magnetic folder 96 is translated to rotation of the needle pin 80.

The screw top 90 may be calibrated such that a one full rotation of the screw top 90 is associated with a defined linear distance. For example and without limitation, the screw top 90 may be calibrated to 1 millimeter per full rotation such that when the screw top 90 is rotated once, the screw 92 comprising the distal end 44 of the needle pin 80 is advanced 1 millimeter into the indentation 88. In this manner, a clinician can easily identify the distance at which the first and second bars 12, 16 are being held apart by the at least one needle pin 80 based on the number of rotations made by the screw top 90.

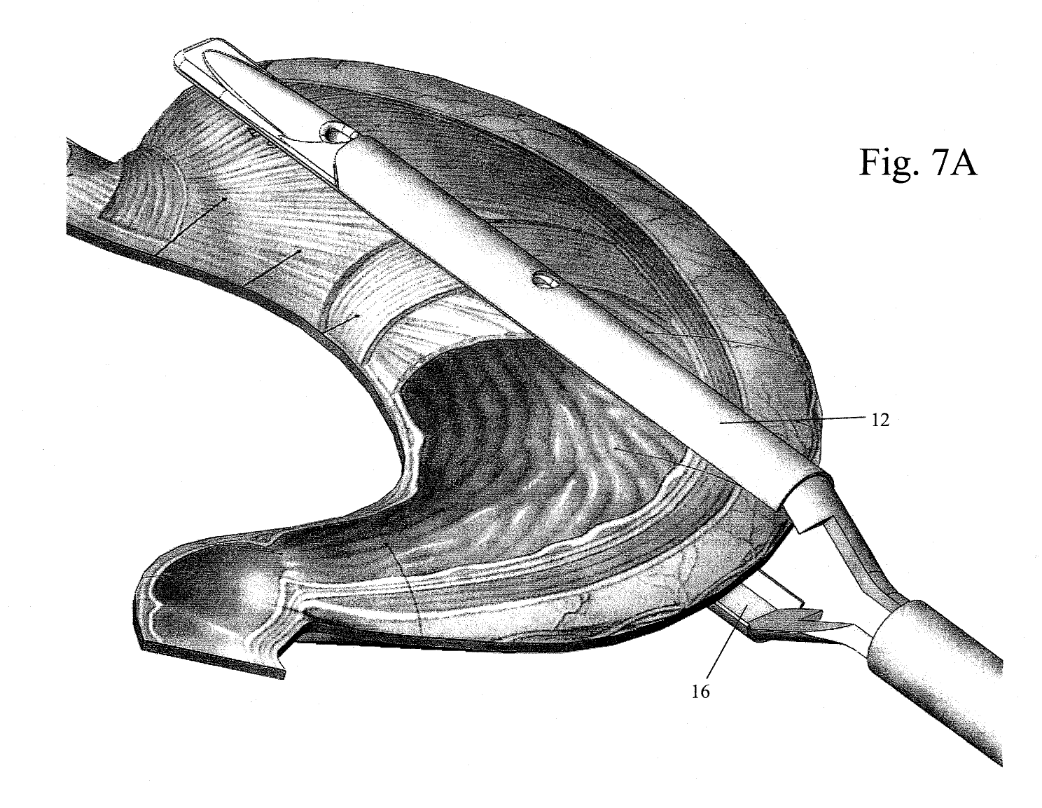

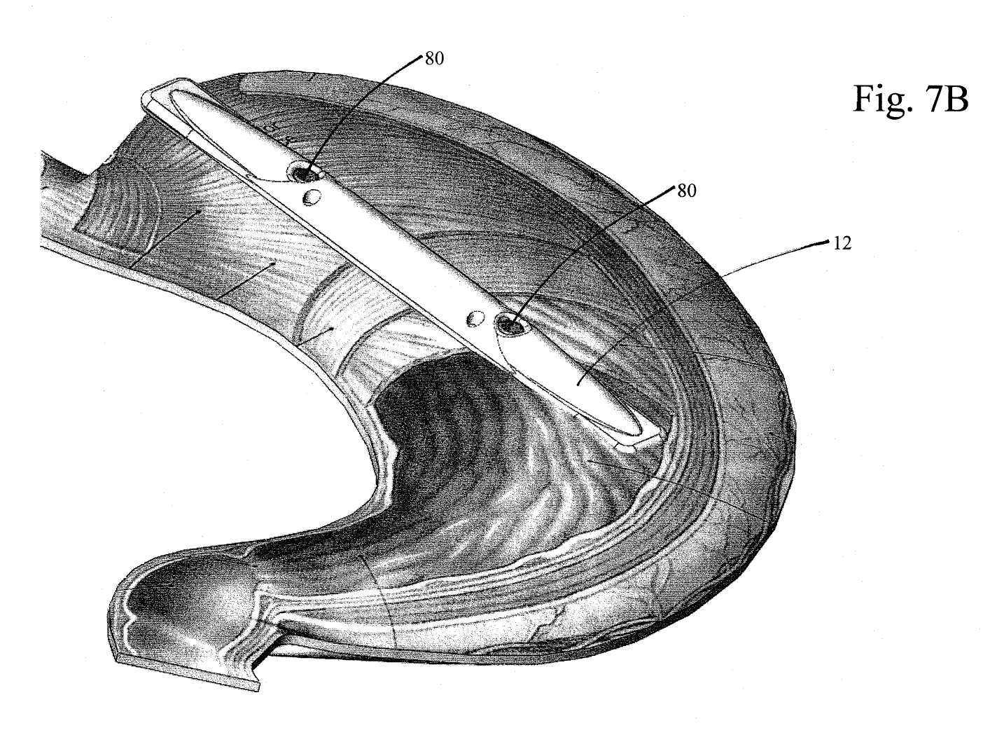

In application, after the first and second components 12, 16 of the remodeling device 10 are properly positioned with respect to a targeted tissue (see FIG. 7A), the needle pins 80 are inserted into the body laparoscopically through a separate port. Accordingly, following the laparoscopic introduction of the needle pins 80, each of the needle pins 80 are inserted into a hole 79 in the first component 12 as shown in FIG. 5B.

Thereafter, downward pressure is applied to the proximal ends 42 of the needle pins 80 such that the distal ends 44 of the needle pins 80 puncture the underlying tissue and engage with the first side 16A of the second component 16. Any standard laparoscopic instrument can be used to apply this downward pressure to the proximal ends 42 of the needle pins 80.

After the needle pins 80 are fully inserted within the holes 79 of the first component 12 (see FIG. 7B), the screw bar 86 is laparoscopically advanced through the body, inserted into the opening in the screw nut 82, and advanced through the hollow interior of the body of the needle pin 80 such that the screw 84 of the distal end 44 threadedly engages the indentation 88 in the second component 16. Upon making contact with the indentation 88 of the second component 16, the rotation of the screw bar 86 is carefully monitored in order to precisely approximate the distance between the first and second components 12, 16. After the prescribed distance between the first and second plates has been achieved, the screw bar 86 may be cut at the superior level of the screw nut 82 via a laparoscopic scissor instrument or otherwise.

Alternatively, in the at least one embodiment of the needle pin 80 wherein the proximal end 42 of the needle pin 80 comprises a screw top 90 configured to receive a twister or torque catheter 94, a twister or torque catheter 94 may be advanced laparoscopically through the body, mated with the screw top 90 of the needle pin 80 and used to rotate the needle pin 80 within the hole 79 such that the distal end 44 of the needle pin 80 comprising the screw 92 is more deeply seated within the indentation 88 of the second component 16. In yet the additional embodiment wherein the proximal end 42 of the needle pin 80 comprises a screw top 90 configured to magnetically engage and be encompassed by the magnetic folder 96, the magnetic folder 96 may be laparoscopically delivered adjacent to the first component 12 of the remodeling device 10, magnetically engaged with the screw top 90 of the needle pin 80 and used to rotate the needle pin. 80 within the hole 79 such that the distal end 44 of the needle pin 80 comprising the screw 92 is more deeply seated within the indentation 88 of the second component 16. In both of the aforementioned embodiments, upon the desired approximation of the first and second components 12, 16 of the remodeling device 10, the twister or torque catheter 94 or, alternatively, the magnetic folder 96 may simply be withdrawn through the abdominal port. Regardless of the configuration of the needle pin 80, in each of the described embodiments, the result of causing the rotation of the needle pin 80 (or screw bar 86) is that the distance between the first and second bars 12, 16 can be precisely fixed through an entirely laparoscopic procedure.

Now referring to FIG. 6B, an alternative embodiment of the needle pin 80 configuration of the at least one pin 22 is shown. In this at least one embodiment, while the needle pin 80 still comprises a proximal end 42, a distal end 44, and a body extending therebetween, both the proximal and distal ends 42, 44 of the needle pin 80 are not open. Rather, in this at least one embodiment, the proximal end 42 of the needle pin 80 comprises a screw top 90 and the distal end 44 of the needle pin 80 comprises a screw 92. In the at least one embodiment shown in FIG. 6B, the screw 92 of the distal end 44 of the needle pin 80 comprises a conical configuration; however, it will be appreciated that it may comprise any configuration so long as the screw 92 is capable of threaded engagement with an indentation 88 disposed in the first side 16A of the second component 16.

Similar to the prior embodiments described with respect to the needle pin 80, the screw top 90 of the proximal end 42 of the needle pin 80 has a diameter that is larger than the holes 79 such that the needle pin 80 is prevented from fully traversing the hole 79 in which it is inserted. The screw top 90 may further be configured such that its proximal-most portion comprises a receptacle configured to receive a magnetic twister or torque catheter 94 therein. Additionally, the screw top 90 may be comprised of a ferromagnetic material to facilitate interaction between the screw top 90 and the twister or torque catheter 94.

In at least one embodiment, the screw top 90 may be calibrated such that a one full rotation of the screw top 90 is associated with a defined linear distance. For example and without limitation, the screw top 90 may be calibrated to 1 millimeter per full rotation such that when the screw top 90 is rotated once, the screw 92 comprising the distal end 44 of the needle pin 80 is advanced 1 millimeter into the indentation 88.

In application, when the at least one needle pin 80 is inserted into a hole 79 in the first component 12, a twister or torque catheter 94 may be advanced laparoscopically through the body, mated with the screw top 90 of the needle pin 80 and used to rotate the needle pin 80 within the hole 79. In this manner, the distal end 44 of the needle pin 80 comprising a screw 92 in threaded engagement with the indentation 88 of the second component 16 may be more deeply seated within the indentation 88 with every rotation of the screw top 90. Similar to the previous embodiments of the needle pin 80 described, due to the calibration of the screw top 90, a clinician can easily calculate the distance at which the first and second bars 12, 16 are being held apart by the at least one needle pin 80 based on the number of rotations made by the screw top 90.

Referring back to the at least one pin 22 of the remodeling device 10 generally, the at least one pin 22 may comprise any length so long as the pin 22 is of a sufficient size to move through a laparoscopic port and is capable of holding the first component 12 and the second component 16 a distance apart when the first and second components 12, 16 are mechanically engaged. In at least one embodiment, each of the at least one pins 22 is about 7 to about 16 millimeters long. As previously noted, when the first side 12A of the first component 12 is mechanically engaged with the first side 16A of the second component 16 through the at least one pin 22, the at least one pin 22 functions to maintain the first component 12 a target distance from the second component 16.

Now referring back to the embodiments of the remodeling device 10 shown in FIGS. 1A-1D, when the first and second components 12, 16 are mechanically engaged with one another via the at least one pin 22, the distal end 44 of the at least one pin 22 is mechanically secured within the second component 16. In this manner, the at least one pin 22 secures the first component 12 to the second component 16 such that an interior space 70 is formed therebetween (similar to as described in connection with the independent pin 22 embodiments). When the remodeling device 10 is applied to a tissue of interest, any tissue disposed between the first and second components 12, 16 is punctured by the at least one pin 22 and gently compressed between the first and second components 12, 16 of the remodeling device 10. Because the at least one pin 22 bears the majority of the compressional load exerted on the underlying tissue, the remodeling device 10 prevents overcompression of the tissue and dehiscence of the underlying tissue.

The interior space 70 comprises a depth that somewhat correlates with the length of the at least one pin 22 of the remodeling device 10. It will be understood that the size of the interior space 70 can be manipulated by a clinician depending on the thickness of tissue and/or organ to be treated or other factors. For example, to achieve a larger interior space 70, the length of the at least one pin 22 may be increased and/or the thickness of the first and second components 12, 16 may be adjusted. Additionally, in certain embodiments, the engagement mechanism used to secure the distal end 44 of the at least one pin 22 to the opposite component 12, 16 (described in more detail below) can be manipulated to achieve the desired size of the interior space 70. Accordingly, a clinician can easily modify the remodeling device 10 such that it may be optimally configured for a particular application on a particular tissue.

In addition to maintaining an interior space 70 between the components 12, 16 when the components 12, 16 are mechanically engaged with each other, the at least one pin 22 of the remodeling device 10 additionally functions to secure the remodeling device 10 to the underlying tissue and secure the mechanical engagement between the first and second components 12, 16. In other words, the one or more pins 22 of the remodeling device 10 enable the remodeling device 10 to remain securely implanted in the appropriate position and thus resist being shifted or becoming dislodged from its original site of implantation.

To facilitate a secure engagement between the one or more pins 22 and the opposite component 12, 16, an engagement mechanism 28 may be employed. While the engagement mechanism 28 is shown in FIGS. 1A-1D in connection with the second component 16, it will be appreciated that the engagement mechanism 28 can be used in connection with the first component 12 exclusively, or with both the first and second components 12, 16 concurrently, depending upon the desired configuration of the remodeling device 10 and the number and placement of the at least one pin 22.

The engagement mechanism 28 functions to receive the distal end 44 of each of the at least one pins 22 such that the same can not be easily released without further manipulation on the part of the clinician. In the at least one embodiment of the engagement mechanism 28 illustrated in FIGS. 1A-1D, the engagement mechanism 28 comprises at least one elongated opening 29 disposed in the first side 16A of the second component 16. Further, the elongated opening 29 comprises an interior area 30 for receiving the distal end 44 of at least one pin 22.

The elongated opening 29 may extend along the entire length of the first side 16A of the second component 16 or along only a portion thereof, so long as the interior area 30 of the elongated opening 29 is capable of receiving the distal end 44 of the at least one pin 22 when the first component 12 is aligned with respect to the second component 16. Alternatively, more than one elongated openings 29 may be disposed within the first side 16A of the second component 16 if the first component 12 comprises more than one pin 22.

The width of the elongated opening 29 is slightly less than the diameter of the at least one pin 22 of the remodeling device 10. Accordingly, when the distal end 44 of the at least one pin 22 is forced into the interior area 30 of the elongated opening 29, the elongated opening 29 and therefore the first side 16A of the second component 16 are locally deformed to accommodate the distal end 44 of the pin 22 (see FIG. 1B). This results in a tight fit between the distal end 44 of the pin 22 and the portion of the elongated opening 29 receiving the same. In this manner, when the distal end 44 of the pin 22 is lodged within the elongated opening 29, the first and second components 12, 16 are securely held in position relative to each other and the interior space 70 is formed. While the strength of the engagement between the first and second components 12, 16 may vary depending on the materials used, in at least one embodiment, the first and second components are capable of withstanding about 5 pounds of force without becoming disengaged.

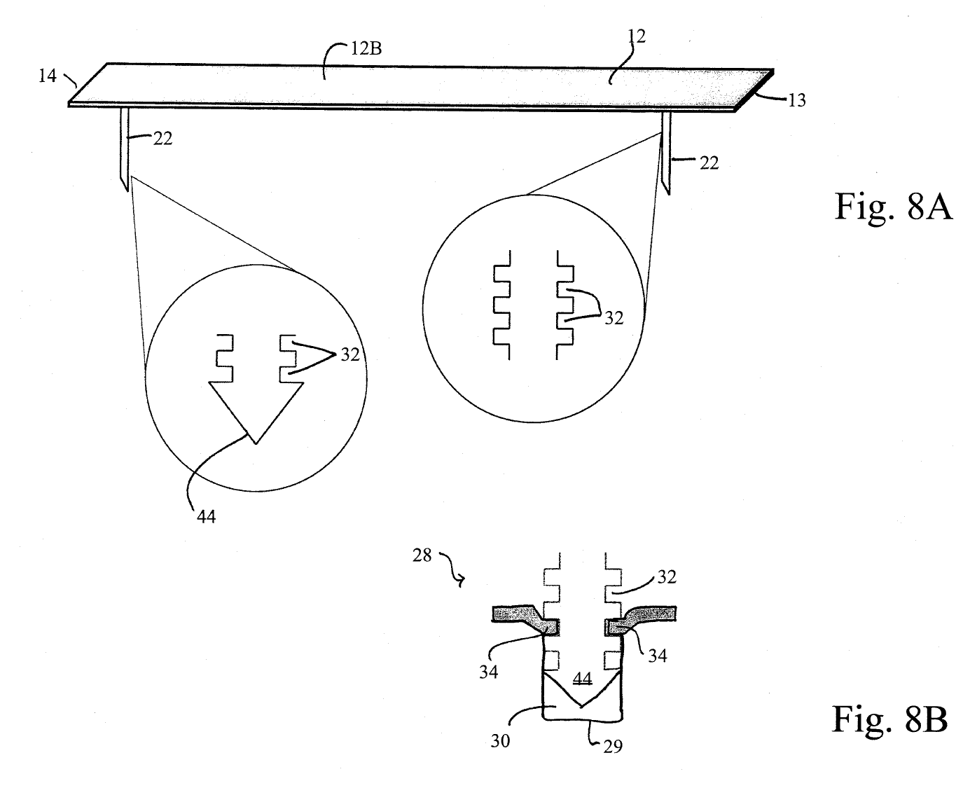

Now referring to FIGS. 8A and 8B, the engagement mechanism 28 may further be configured to provide additional support for the mechanical engagement between the distal end 44 of the pin 22 and the elongated opening 29. For example and without limitation, the portion of the edge defining the elongated opening 29 of the engagement mechanism 28 may comprise a lip 34 that extends into the interior area 30 of the elongated opening 29 as shown in FIG. 8B. Due to the configuration and placement of the lips 34 with respect to the elongated opening 29, in this at least one embodiment, the lips 34 narrow the initial width of the elongated opening 29 such that its diameter is even narrower than initially described.

In at least one embodiment, the at least one pin 22 additionally comprises one or more notches 32 formed around its perimeter (see FIG. 8A). The notches 32 of the pin 22 are configured to receive the lips 34 that extend from the edge of the elongated opening 29 when the distal end 44 of the pin 22 is inserted into the interior area 30 of the elongated opening 29. While an initial amount of force is required to initially insert the distal end 44 of the pin 22 into the narrowed width of the elongated opening 29, after the pin 22 is advanced such that the distal-most notch 32 on the pin 22 receives the lips 34, the distal end 44 of the at least one pin 22 is held comfortably therein without stressing or locally deforming the elongated opening 29.

The dimension(s) of the one or more notches 32 of the pin 22 may be specifically configured in order to facilitate the proper sizing of the interior space 70 between the first and second components 12, 16. For example, because the distal-most notch 32 of a pin 22 is located a set distance from the proximal end 42 of the pin 22, a clinician can easily determine the width of the interior space 70 between the first and second components 12, 16 when the lips 34 of the elongated opening 29 are engaged with the distal-most notch 32 of the pin 22. This is particularly helpful with respect to laparoscopic introductions of the remodeling device 10 as a clinician can immediately obtain the size of the interior space 70 without performing any difficult and/or time-consuming measuring procedures.

Furthermore, in an embodiment where the at least one pin 22 comprises more than one notch 32 disposed thereon, should the clinician desire to decrease the width of the interior space 70, the clinician can simply apply downward pressure to the pin 22 in order to drive the distal end 44 of the pin 22 deeper into the interior area 30 of the elongated opening 29. If the at least one pin 22 comprises more than one notches disposed thereon, the pin 22 can then be forced into the interior space 30 until the next subsequent notch 32 of the pin 22 is received by the lips 34. As the distance between each notch 32 of the pin 22 is predetermined, a clinician can easily and accurately decrease or increase the width of the interior space 70 simply by applying pressure and either withdrawing the pin 22 from or advanced the pin 22 into the interior area 30 of the elongated opening 29. Accordingly, the engagement mechanism 28 in this at least one embodiment functions not only to facilitate a secured mechanical engagement between the first and second components 12, 16, but also to allow a clinician to easily identify the width of the interior spaced 70 formed between the first and second components 12, 16 even when the remodeling device 10 is implanted via laparoscopic delivery.

In each of the embodiments of the engagement mechanism 28 heretofore described, the engagement mechanism 28 is capable of facilitating a secure, albeit releasable, engagement between the first and second components 12, 16 via the at least one pin 22. The remodeling procedure performed through the use of the remodeling device 10 can be easily reversed and the remodeling device 10 can be removed from the patient's body through a minimally invasive procedure. In at least one embodiment of the remodeling device 10 described in connection with FIGS. 1A-1D, the engagement mechanism 28 may further comprise a first plate 40 and a second plate 41 to facilitate the ease with which a clinician can laparoscopically release the engagement between the first and second components 12, 16 of the remodeling device 10.

FIGS. 1A, 1B and 1D show the first plate 40 and the second plate 41 coupled with the sides of the second component 16. Specifically, the first and second plates 40, 41 are coupled with the lateral sides of the second component 16 such that the first and second plates 40, 41 are positioned opposite of each other and the lateral axis of the elongated opening 29 extends along the first side 16A of the second component 16 therebetween. Further, the first and second plates 40, 41 are positioned at a location on the second component 16 such that when the at least one pin 22 of the first component 12 is lodged within the elongated opening 29, the pin 22 is not positioned at a point directly between the first and second plates 40, 41.

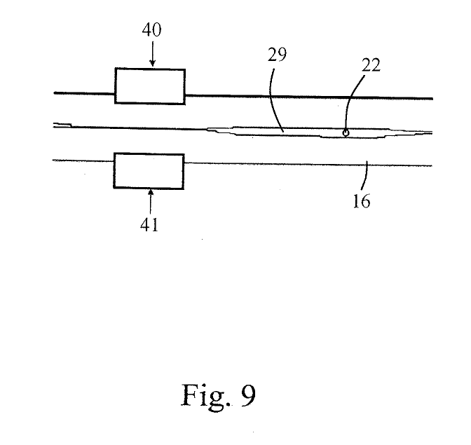

Because the elongated opening 29 that extends along the first side 16A of the second component 16 forms the interior area 30, the elongated opening 29 represents a weakened portion of the second component 16. When a force is applied to both the first and second plates 40, 41 in a direction that is substantially perpendicular to the linear axis of the elongated opening 29 (as illustrated in FIG. 9), the force causes the portion of the elongated opening 29 extending between the two plates 40, 41 to become compressed. Accordingly, force exerted in this manner on the first and second plates 40, 41 translates to a compressional force that is capable of bending the portion of the elongated opening 29 positioned immediately between the first and second plates 40, 41 and thereby decreasing the related interior area 30 thereof.

When a portion of the elongated opening 29 is compressed and bent, the interior area 30 that is immediately adjacent to the bent portion of the elongated opening 29 is increased to accommodate the adjacent compression (see FIG. 9). In this manner, the width of the adjacent portion of the elongated opening 29 may be increased to a size greater than the diameter of the distal end 44 of the pin 22. Accordingly, in at least one embodiment, the first and second plates 40, 41 are positioned on the second component 16 in a location adjacent to where the at least one pin 22 of the first component is inserted into the elongated opening 29. In this manner, when force is applied to the first and second plates 40, 41 such that the portion of the elongated opening 29 disposed between the plates 40, 41 is compressed, the adjacent area of the elongated opening 29 is increased in size which thus releases the pin 22 therefrom. Accordingly, through applying a sufficient force to both the first and second plates 40, 41, the engagement mechanism 28 of the remodeling device 10 is capable of releasing the at least one pin 22 coupled therewith, which allows for the simple removal the remodeling device 10 from a targeted tissue.

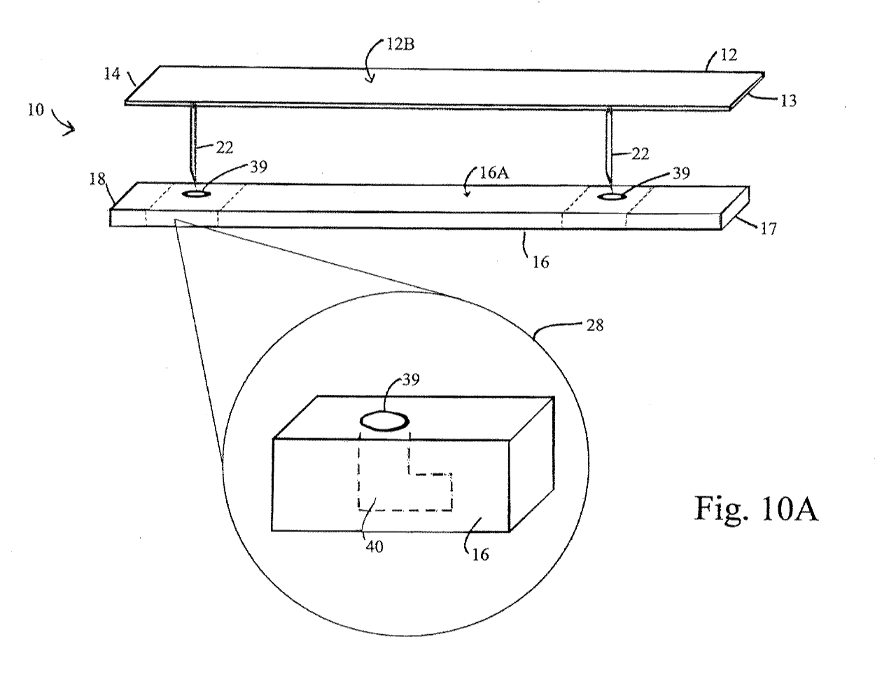

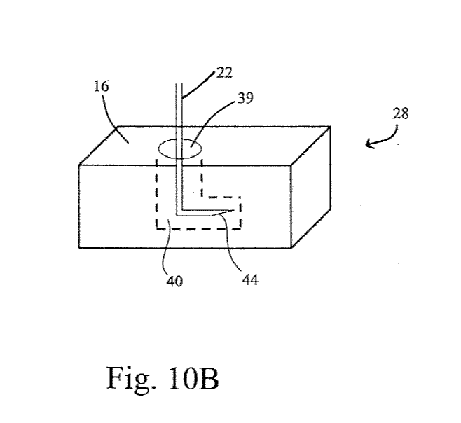

Now referring to FIGS. 10A and 10B, at least one alternative embodiment of the engagement mechanism 28 of the remodeling device 10 is shown. In this at least one embodiment, the engagement mechanism 28 comprises at least one opening 39 in the first side 16A of the second component 16, and an interior area 40 in communication therewith. The interior area 40 may be configured in any manner, so long as the shape of the interior area 40 comprises an angle or bend. For example and without limitation, the interior area 40 shown in FIGS. 10A and 10B comprises an L-shape. While engagement mechanism 28 shown in FIG. 10A comprises two openings 39 and two interior areas 40 disposed in the first side 16A of the second component 16, it will be understood that the engagement mechanism 28 may comprise any number of openings 39 and related interior areas 40 disposed on either or both of the first and second components 12, 16.

In this at least one embodiment of the engagement mechanism 28, the at least one opening 39 is configured to receive the distal end 44 of the at least one pin 22 when the first and second components 12, 16 are mechanically engaged. Both the opening 39 and the interior area 40 comprise diameters that are larger than the diameter of the at least one pin 22. Accordingly, in this at least one embodiment, the distal end 44 of the at least one pin 22 need not be forced through the at least one opening 39 of the engagement mechanism 28. As such, a clinician delivering the remodeling device 10 to a targeted tissue can easily insert the distal end 44 of the at least one pin 22 into the at least one opening 39 of the engagement mechanism 28 through laparoscopic means or otherwise.

Similar to the manner in which a deployed staple is prevented from being removed through the hole in which it is inserted, the angled or bent configuration of the interior area 40 is capable of facilitating the retention of the distal end 44 of the at least one pin 22 therein. In this at least one embodiment, the distal end 44 of the pin 22 may be configured such that when enough pressure is applied, the distal end 44 is capable of bending. As shown in FIG. 10B, when the distal end 44 of the at least one pin 22 is advanced into the bent or angled interior area 40 of the engagement mechanism 28 and a sufficient amount of downward force is applied, the at least one pin 22 assumes a bent configuration. In this manner, the at least one pin 22 of the remodeling device 10 serves to retain the distal end 44 of the pin 22 within the interior area 40 and thereby enables the first and second components 12, 16 to remain securely mechanically engaged with one another even when opposing pressure is applied.

As with previously described embodiments of the engagement mechanism 28, this at least one embodiment of the engagement mechanism 28 is also reversible. Here, a remodeling procedure performed through use of the remodeling device 10 can be easily reversed by simply using enough force to separate the first and second components 12, 16 such that the distal end 44 of the pin 22 unbends. For example, and without limitation, when the first component 12 is mechanically engaged with the second component 16 via the engagement mechanism 28, a clinician can pull the first component 12 away from the second component 16 with a sufficient force such that the distal end 44 of the pin 22 is forced into a substantially straightened configuration and is easily withdrawn from the at least one opening 39.

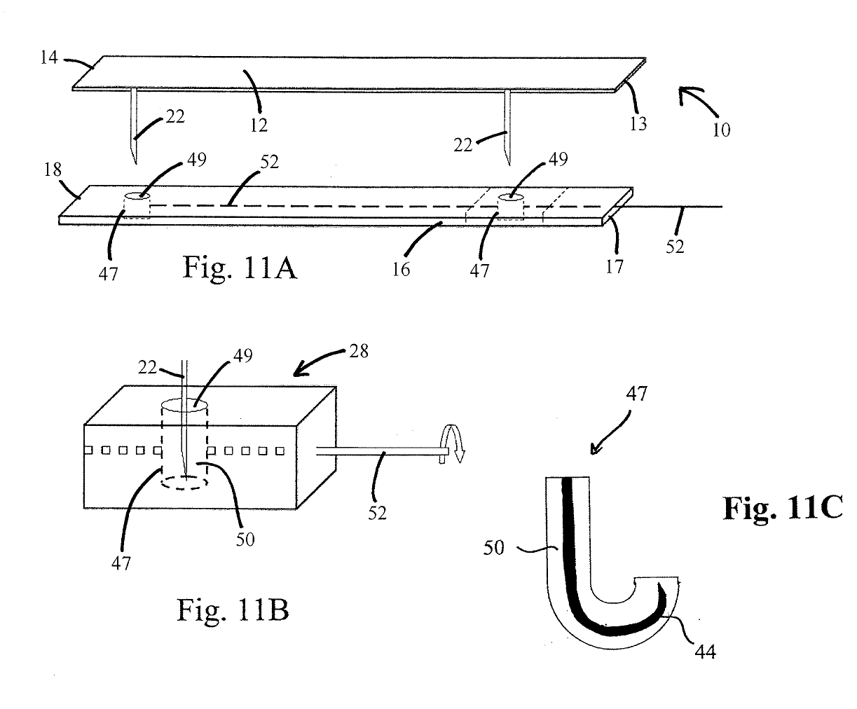

Referring now to FIGS. 11A-11C, an additional embodiment of the engagement mechanism 28 of the remodeling device 10 is shown. In this at least one embodiment, the engagement mechanism 28 comprises at least one opening 49 in the first side 16A of the second component 16 and at least one receptacle 47. Each receptacle 47 of the engagement mechanism 28 defines an interior area 50 and is in communication with at least one of the openings 49. As shown in FIG. 11B, the receptacle 47 at least initially comprises a straight configuration and, for example, may be cylindrical in nature. Furthermore, the receptacle 47 may be formed of a flexible or semi-flexible material such that its shape is capable of being manipulated, thereby altering the shape of the related interior area 50. While the engagement mechanism 28 shown in FIGS. 11A and 11B comprises two openings 49, two receptacles 47 and two interior areas 50, it will be understood that the engagement mechanism 28 may comprise any number of openings 49 and related receptacles 47 and interior areas 50, and may be disposed on either or both of the first and second components 12, 16.

In this at least one embodiment of the engagement mechanism 28, the second component 16 of the remodeling device 10 additionally comprises a rotatable shaft 52 extending between the proximal and distal ends 17, 18 of the second component 16. Each of the receptacles 47 of the engagement mechanism 28 is mounted on the rotatable shaft 52 such that, when the shaft 52 is rotated, the shape of at least a portion of each of the receptacles 47 is manipulated. More specifically, due to the interaction between the shaft 52 and the receptacle(s) 47, rotation of the shaft 52 causes the one or more receptacles 47 of the engagement mechanism 28 to similarly rotate. As a result, the interior area(s) 50 associated with each receptacle 47 is caused to transform from a substantially cylindrical configuration to a configuration comprising a curve (see FIG. 11C).

The rotatable shaft 52 may extend a distance beyond the proximal end 17 of the second component 16 such that the rotatable shaft 52 may be easily rotated using a laparoscopic device or otherwise. In addition, the rotatable shaft 52 may be comprised of titanium, stainless steel or any other material that is capable of being rotated in clockwise and counterclockwise directions. Both the at least one opening 49 and the related interior area 50 have diameters that are greater than the diameter of the distal end 44 of the at least one pin 22 and are configured to slidably receive the distal end 44 of the at least one pin 22 therein. Accordingly, similar to the embodiments of the engagement mechanism 28 described in connection with FIGS. 10A and 10B, the distal end 44 of the at least one pin 22 need not be forced through the at least one opening 49 or the interior area 50 of the engagement mechanism 28, which facilitates ease of delivery of the remodeling device 10 to a targeted tissue.

In this at least one embodiment, the engagement mechanism 28 utilizes the rotation of the shaft 52 and the receptacle(s) 47 to lock the distal end 44 of the at least one pin 22 within the related interior area 50. Specifically, when the distal end 44 of the at least one pin 22 is positioned within the interior area 50 of a receptacle 47 and the shaft 52 is rotated, a rotational force is applied to the distal end 44 of the at least one pin 22. As shown in FIG. 11C, the application of this rotational force to the distal end 44 of the pin 22 causes the distal end 44 of the pin 22 to bend in order to conform with the curved configuration of the interior space 50 in which it is inserted. In this manner, this at least one embodiment of the engagement mechanism 28 is capable of bending the distal end 44 of the at least one pin 22 into a substantially hooked, curved or arched configuration that is embedded within the similarly configured interior space 50. Accordingly, due to the curved nature of the distal end 44 of the pin 22, the distal end 44 of the pin 22 resists removal from the receptacle 47 in which it is inserted and thereby functions to mechanically secure the first and second components 12, 16 of the remodeling device 10 together. Furthermore, in at least one embodiment, due to the force required to straighten the distal end 44 of the pin 22 once it has been manipulated into a curved and/or bent configuration, the inadvertent back rotation of shaft 52 and thus the receptacle 47 in which the pin(s) 22 is/are embedded is prevented.

In the event it is desirable for the first and second components 12, 16 to become disengaged from one another, the curved configuration of the distal end 44 of the at least one pin 22 may be easily reversed. For example, in at least one embodiment, to reverse the secured engagement between the distal end 44 of the pin 22 and the interior space 50 of the engagement mechanism 28, a sufficient rotational force may be applied to the rotatable shaft 52 in a direction that is opposite of the curved or bent configuration of the receptacle 47 and the distal end 44 of the pin 22. In this manner, the rotatable shaft 52 causes the receptacle 47 and related interior area 50 to rotate back into a substantially cylindrical configuration, thereby unbending the distal end 44 of the at least one pin 22. Accordingly, when the distal end 44 of the pin 22 again comprises a substantially straight configuration, the pin 22 may be easily withdrawn from the interior space 50 through the opening 49, thereby releasing the first component 12 from the second component 16.

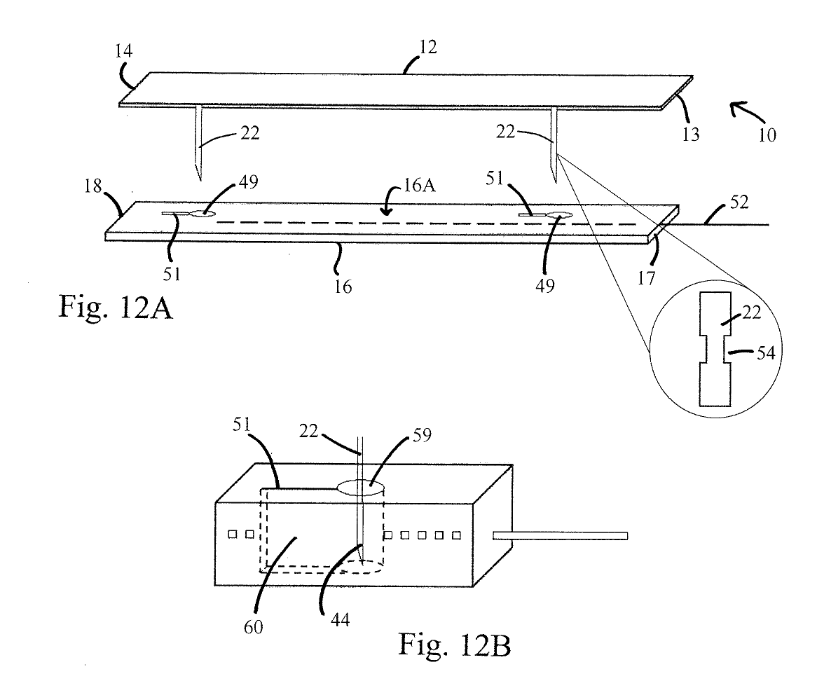

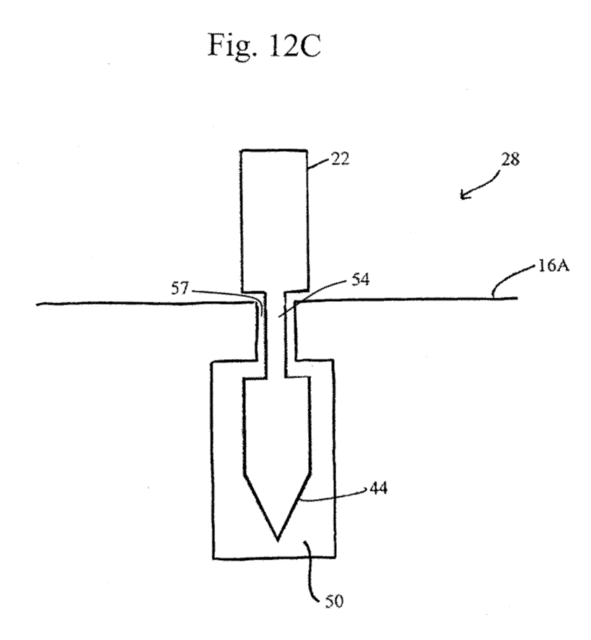

In yet another alternative embodiment of the engagement mechanism 28, the engagement mechanism 28 may comprise at least one opening 49 in the first side 16A of the second component 16, an interior area 50 in communication therewith, and a slot 51 coupled with both the at least one opening 49 and the interior area 50 as shown in FIGS. 12A-12C. While the engagement mechanism 28 shown in FIG. 12A comprises two openings 49, two interior areas 50, and two slots 51 disposed in the first side 16A of the second component 16, it will be understood that the engagement mechanism 28 may comprise any number of openings 49, related interior areas 50, and slots 51 disposed on either or both of the first and second components 12, 16.

In this at least one embodiment of the engagement mechanism 28, the at least one opening 49 and the related interior area 50 comprise diameters that are greater than the largest diameter of the distal end 44 of the at least one pin 22. In addition, the at least one opening 49 and the related interior area 50 are configured to slidably receive the distal end 44 of the at least one pin 22 therein when the first and second components 12, 16 of the remodeling device 10 are mechanically engaged. Accordingly, in this at least one embodiment, the distal end 44 of the at least one pin 22 need not be forced through the at least one opening 49 of the engagement mechanism 28 and, as such, a clinician delivering the remodeling device 10 to a targeted tissue can easily insert the distal end 44 of the at least one pin 22 into the at least one opening 49 of the engagement mechanism 28 through laparoscopic means or otherwise. However, the width of the slit 51 does comprise a diameter that is less than the largest diameter of the distal end 44 of the pin 22.

In this at least one embodiment of the engagement mechanism 28, the pin 22 further comprises one or more notches 54 disposed thereon as shown in FIG. 12A. The notches 54 function to decrease the diameter of the pin 22 in specific locations along its length such that the diameter of the pin 22 at each of the one or more notches 54 is less than the width of the slit 51 of the engagement mechanism 28. Accordingly, as shown in FIG. 12B, the distal end 44 of the pin 22 may be inserted through the opening 49 and into the portion of interior space 50 in communication therewith. Thereafter, the at least one notch 54 of the pin 22 is aligned with the slit 51 and the pin 22 can be slid away from the opening 49 and into the slit 51 such that the notch 54 receives the edges of the slit 51 therein (see FIG. 12C showing a cross-section view of the interaction). Because the diameter of the pin 22 immediately adjacent to the notch 54 is greater than the width of the slit 51, the distal end 44 of the pin 22 is thus secured within the engagement mechanism 28 of the remodeling device 10 and functions to hold the first and second components 12, 16 of the remodeling device 10 in mechanical engagement with each other.

Similar to the one or more notches 32 described in connection with the at least one embodiment of the engagement mechanism 28 shown in FIGS. 8A and 8B, the dimension(s) of the one or more notches 54 of the pin 22 may be specifically configured in order to facilitate the proper sizing of the interior space 70 between the first and second components 12, 16. For example, because the distal-most notch 54 of a pin 22 is located a set distance from the proximal end 42 of the pin 22, a clinician can easily determine the width of the interior space 70 between the first and second components 12, 16 when the distal-most notch 54 is engaged with the slit 54 of the engagement mechanism 28. Furthermore, in an embodiment where the at least one pin 22 comprises more than one notch 54 disposed thereon, should a clinician desire to decrease the width of the interior space 70, the clinician can simply engage the next subsequent notch 54 located on the pin 22 with the slit 51 of the engagement mechanism 28. As the distance between each notch 54 of the pin 22 is predetermined, a clinician can easily and accurately decrease or increase the width of the interior space 70 simply by engaging the proper notch 54 on the pin 22 with the slit 51. Accordingly, in this at least one embodiment, the engagement mechanism 28 functions not only to facilitate a secured mechanical engagement between the first and second components 12, 16, but also allows a clinician to easily identify the width of the interior spaced 70 formed between the first and second components 12, 16 even when the remodeling device 10 is implanted via laparoscopic delivery. Additionally, while the engagement mechanism 28 of this at least one embodiment facilitates a secure mechanical engagement between the first and second components 12, 16, should it be desirable to reverse the remodeling procedure, a clinician need only slidably move the first component 12 relative to the second component 16 such that the pin 22 slides through the slit 51 in a direction toward the related opening 49. In this manner, the notch 54 of the pin 22 that is engaged with the slit 51 of the engagement mechanism 28 can be slidably removed from the slit 51, and the distal end 44 of the pin 22 can be easily withdrawn through the opening 49.

In operation, the remodeling device 10 may be applied to an organ or tissue of interest in order to remodel the underlying tissue or organ into a desired configuration and/or provide support to the same. As will be discussed in further detail below, the remodeling device 10 may be used for temporary or chronic implantation within a body without the risk of the first and second components 12, 16 migrating through the underlying tissue. Furthermore, because the remodeling device 10 does not require sutures or staples to achieve remodeling or provide support to the tissue of interest, implantation of the remodeling device 10 is entirely reversible and, if desired, the remodeling device 10 may be easily removed from the organ or tissue of interest through a laparoscopic procedure.

As previously described, the specifications of the remodeling device 10 may be modified to achieve a desired result. For example, and without limitation, the dimensions of the components 12, 16 and/or the one or more pins 22 may be chosen for a particular application for which the remodeling device 10 is to be used and/or based on the patient. In addition, the particulars of the engagement mechanism 28 utilized as well as the number of pins 22 employed may also be determined pursuant to the needs of the patient and/or application of the remodeling device 10. Accordingly, while certain embodiments of the remodeling device 10 may be described in connection with particular tissues or organs, it will be appreciated that any of the embodiments of the remodeling device 10 described herein may also be applied to any tissue or organ of interest in a similar manner and use of the particular embodiments of the remodeling device 10 in lieu of others may be determined based on the patient's specifications, the specific application, and/or the tissue or organ in question.

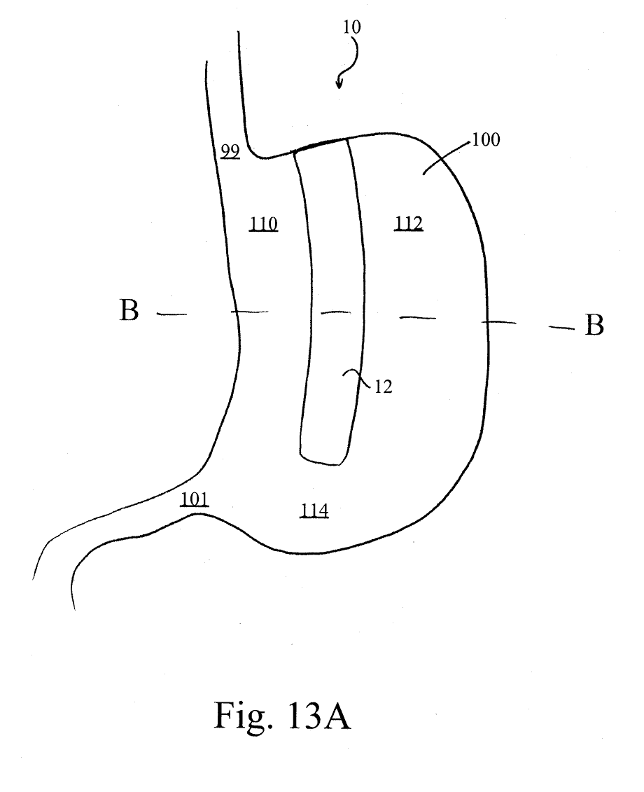

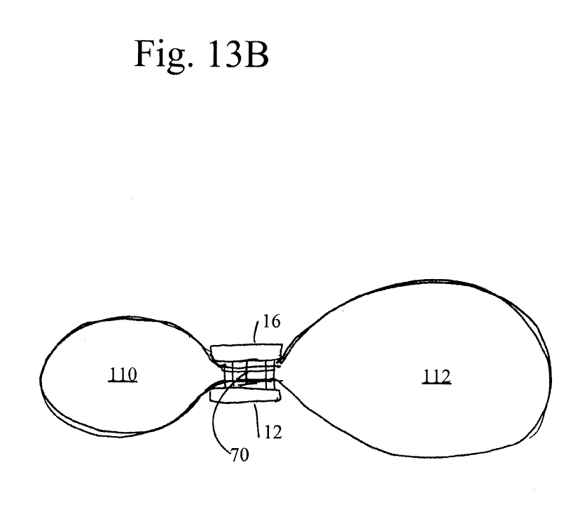

In operation, the remodeling device 10 may be applied to a stomach 100 as shown in FIG. 13A. Specifically, in this at least one embodiment, the first side 12A of the first component 12 is positioned adjacent to the anterior wall of the stomach 100 and the first side 16A of the second bar 16 is positioned adjacent to the posterior wall of the stomach 100. While the remodeling device 10 is shown in FIG. 13A in a vertical placement with respect to the stomach 100, it will be understood that the remodeling device 10 may alternatively be positioned in a horizontal configuration or an angular configuration with respect to the stomach 100.

After the first and second components 12, 16 are positioned in the desired location with respect to the stomach 100, if the at least one pin 22 is not already extending from the first side 12A of the first component 12, it is either deployed into the substantially extended configuration or inserted through the applicable hole in the first component 12, dependent upon which embodiment of pins 22 are employed. Thereafter, pressure is applied to both the first and second components 12, 16 such that the distal end 44 of the at least one pin 22 pierces the underlying anterior and posterior portions of the stomach tissue 100. When the distal end 44 of the at least one pin 22 is protruding through the posterior stomach wall 100, the distal end 44 mechanically engages the first side 16A of the second component 16 and securely couples therewith. In this manner, the portion of the stomach 100 disposed between the two components 12, 16 is encased within the interior space 70 defined by the at least one pin 22 of the first component 12. Because the at least one pin 22 prevents the remodeling device 10 from overly compressing the underlying tissue and the pin 22 assumes the majority of the compressional force, while this portion of the stomach 100 is somewhat compressed between the two components 12, 16 and thereby remodeled from its normal anatomical shape, the underlying tissue is not overly compressed, thereby preventing the formation of adhesions and/or the permanent remodeling of the tissue.

As shown in FIG. 13B, when the first and second components 10, 12 of the remodeling device 10 are positioned on the stomach 100 and mechanically engaged, the stomach is divided into two portions—one small gastric pouch 110 and one larger, residual stomach portion 112. As the small gastric pouch 110 receives ingested matter directly from the gastroesophageal junction 99, the placement of the remodeling device 10 as shown in FIG. 13A thus inhibits ingested matter from moving into the residual stomach portion 112. Instead, such ingested matter is directed through the smaller gastric pouch 110 and into the pyloric canal 101 where a significant portion of the ingested matter is evacuated from the stomach 100. Due to the size of the small gastric pouch 110, the amount of food that the patient can consume at one time is significantly reduced and satiety is more quickly achieved.

However, because the remodeling device 10 does not extend along the entire length of the stomach 100, an outflow tract 114 is formed caudally of the remodeling device 10. This outflow tract 114 allows an amount of ingested matter received through the gastroesophageal junction 99 to move into the residual gastric pouch 112 in a controlled manner such that the ingested matter can proceed through normal digestion. In addition, the outflow tract 114 allows any food matter or enzymes residing within the residual gastric pouch 112 to evacuate the stomach 100.

While the delineation formed by the remodeling device 10 between the small gastric pouch 110 and the residual gastric pouch 112 is not leak-proof, the mechanical engagement between the first and second bars 12, 16 of the remodeling device 10 provides support to the anterior and posterior walls of the stomach 100 such that the stomach 100 is prevented from distending. In this manner, most of the food matter received into the small gastric pouch 112 through the gastroesophageal junction 99 is maintained therein and the patient exhibits the sensation of satiety earlier.

Now referring to FIGS. 14A and 14B, an additional embodiment of the remodeling device 10 is shown. Remodeling device 200 is configured similarly to the remodeling device 10, with like reference numerals referring to like components. As shown in FIG. 14A, in addition to the first and second components 12, 16, the at least one pin 22 and an engagement mechanism 28, the remodeling device 200 additionally comprises a lace component 260 and at least one spring element 262. The lace component 260 comprises a semi-rigid membrane lace material such as polyurethane, PTFE, silastic, or any other material suitable in the medical arts, and is coupled with both the distal end 14 of the first component 12 and the distal end 18 of the second component 16. The lace component 260 is configured such that when the first and second components 12, 16 are mechanically engaged with each other via the at least one pin 22, the lace component 260 defines an interior space 240.

The at least one spring element 262 of the remodeling device 200 is embedded within the first component 12, the lace component 260 and the second component 16 and is biased to oppose the mechanical engagement of the first and second components 12, 16. The spring element 262 may comprise any degree of flexibility or rigidity. For example, the degree of pliability of the spring element 262 may be determined by a clinician pursuant to the particular application for which the remodeling device 200 is to be used.

Because the spring element 262 is biased to oppose the mechanical engagement of the first and second components 12, 16, the spring element 262 facilitates the delivery of the remodeling device 200 to a targeted tissue by maintaining the first and second components 12, 16 a prescribed distance apart. In this manner, the spring element 262 reduces the chance that the distal ends 44 of the pins 22 of the remodeling device 200 will inadvertently interact with a tissue, be it the targeted tissue or a non-targeted tissue, upon delivery of the device 200.

Furthermore, in at least one embodiment of the remodeling device 200, both the first and second bars 12, 16 may comprise at least one stitch loop 270 extending therefrom (see FIG. 14A). The at least one stitch loop 270 may comprise any configuration so long as the stitch loop 270 is capable of coupling with sutures to assist in securing the remodeling device 200 to the targeted tissue.

In operation, when the first and second components 12, 16 are positioned opposite portions of a targeted tissue, the lace component 260 functions to secure at least the distal ends 14, 18 of the two components 12, 16 together. Similar to the remodeling device 10 as described above, the remodeling device 200 is capable of remodeling the underlying tissue while avoiding constriction and excessive compression thereof. Further, the addition of the lace component 260 allows a portion of the targeted tissue to remain uncompressed or be less compressed than the adjacent tissue disposed between the first and second components 12, 16 of the remodeling device 200.

For example and without limitation, when the remodeling device 200 is used to treat a stomach 100, in at least one embodiment the remodeling device 200 is transversely delivered to the stomach 100 as shown in FIG. 15A. Accordingly, the first and second components 12, 16 are positioned adjacent to the anterior and posterior portions of the stomach 100, respectively, and the lace component 260 is disposed proximate to or around the lesser curvature of the stomach 100. After the at least one pin 22 is mechanically engaged with the opposite component of the remodeling device 200 and the two components 12, 16 are securely coupled as previously described with respect to the remodeling device 10, due to the alternative placement of the device 200 as compared to the previous example, the small gastric pouch 110 is formed above the remodeling device 200 and the residual gastric portion 112 is formed below.

Additionally, the closed loop of the lace component 260 defines the interior space 240. As shown in FIG. 15B, in this application, the interior space 240 defines an outlet 116 from the small gastric pouch 110 so that an outflow of digested matter can flow therethrough and into the lower residual gastric portion 112 of the stomach 100. Accordingly, the size of the interior space 240 affects how quickly food and other digested matter may exit the small gastric pouch 110. The diameter of the outflow tract 116 is directly associated with the size of the interior space 240 formed by the lace component 260 and, as such, modification of the length of the lace component 260 adjusts the resulting diameter of the outflow tract 116. It will be appreciated that a clinician can modify the dimensions of the lace component 260 as is necessary depending on variables such as the precise treatment for which the remodeling device 200 is to be used and the particular patient.

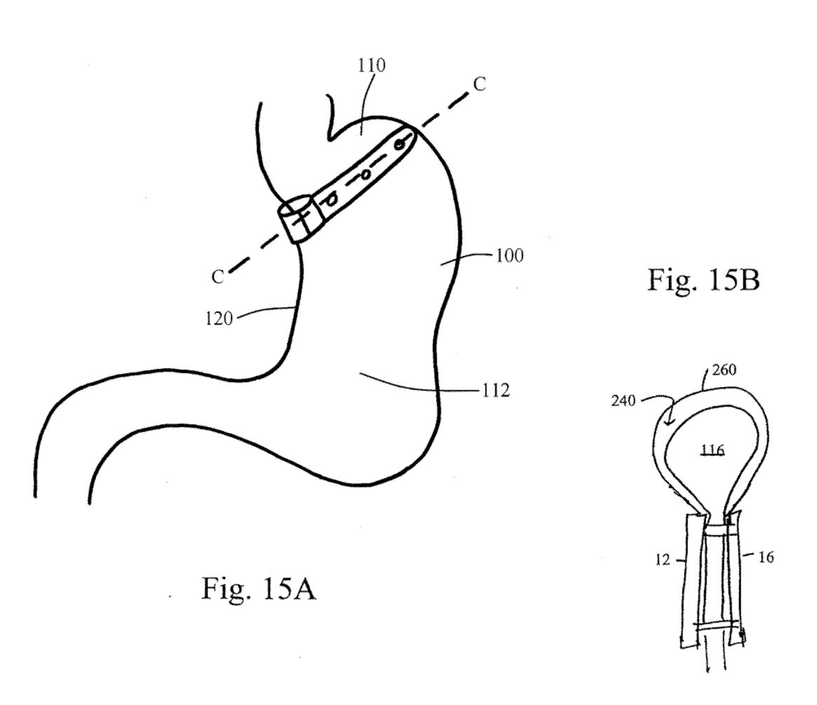

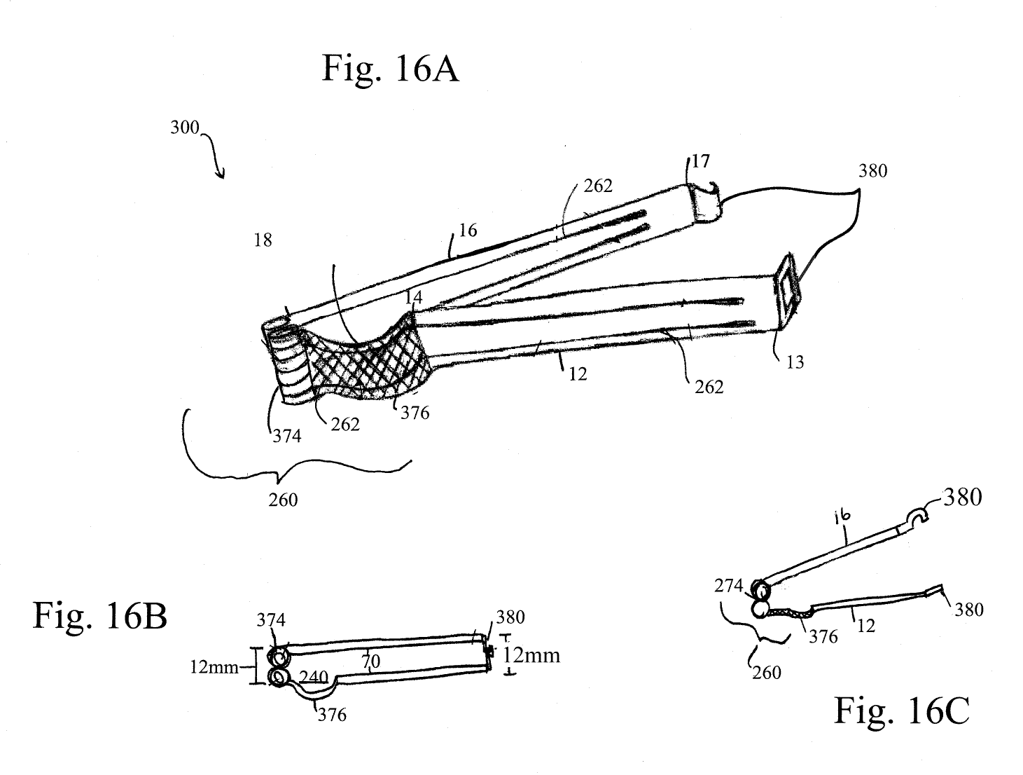

Now referring to FIGS. 16A-16C, at least one alternative embodiment of the remodeling device 200 is shown. Remodeling device 300 is configured similarly to the remodeling device 200 with like reference numerals referring to like components. As shown in FIG. 16A, in addition to the first and second components 12, 16 and the lace component 260, the remodeling device 300 further comprises a latch mechanism 380. In addition, the lace component 260 of the remodeling device 300 further comprises a spring system 374 coupled with a stent-shaped lace component 376. In this at least one embodiment of the remodeling system 300, the inclusion of at least one pin 22 and the engagement mechanism 28 is optional and, as shown in FIGS. 16A-16C, such additional components are not required.

As previously noted, the lace component 260 of the remodeling device 300 further comprises a spring system 374 and a stent-shaped lace component 376. As shown in FIG. 16A, the spring system 374 is coupled with the distal end 18 of the second component 16 and the stent-shaped lace component 376 is coupled with the distal end 14 of the first component 12. The spring system 374 comprises at least two springs positioned adjacent to each other and, in at least one embodiment, is about 12 millimeters wide and about 12 millimeters tall. It will be appreciated that the spring system 374 may comprise any dimensions, provided that the spring system 374 is capable of being inserted through an abdominal port and advanced laparoscopically through the body. Further, in at least one embodiment of the remodeling device 300, the spring system 374 comprises stainless steel, although any similar material may be employed. Furthermore, the spring system 374 is biased such that some movement is possible between the at least two springs of the spring system 374.

The stent-shaped lace component 376 may be comprised of pliable wire tubes, polyurethane, PTFE mesh or any other materials so long as the stent-shaped lace component 376 is at least semi-flexible and capable of being implanted within a body for extended periods of time. The stent-shaped lace component 376 is coupled at its distal end with at least one of the springs of the spring system 374 and coupled at its proximal end with the distal end 14 of the first component 12. Furthermore, the at least one spring element 262 embedded within both the first and second components 12, 16 also runs through the spring system 374 and stent-shaped lace component 376 of the remodeling device 300. As with remodeling device 200, the at least one spring element 262 is biased to oppose mechanical engagement between the first and second components 12, 16 of the remodeling device 300. In this manner, the at least one spring element 262 provides some rigidity to the lace component 260 of the remodeling device 300 and further keeps the lace component 260 in an open configuration, which facilitates delivery of the lace component 260, and ultimately the remodeling device 300, to the targeted tissue.

Additionally, the proximal end 13 of the first component 12 and the proximal end 17 of the second component 16 are coupled with components of a latch mechanism 380. As shown in FIGS. 16A-16C, the latch mechanism 380 may comprise a clip and hook combination or it may be comprised of any other clip or latch mechanism that is easily fastened and capable of maintaining a secured engagement over a period of time. In at least one embodiment, when the latch mechanism 380 is fastened, the latch mechanism 380 comprises a width of about 12 millimeters. In addition to the width of the spring system 374, the width of the fastened latch mechanism 380 assists in defining the interior space 70 between the first and second components 12, 16 (see FIG. 16C). In the at least one embodiment of the remodeling device 300 comprising at least one pin 22 (not shown), when the latch mechanism 380 is fastened, the interior space 70 is additionally defined by the at least one pin 22 in addition to the latch mechanism 380 and the spring system 374.

Due to the flexibility of the spring system 374 and the stent-shaped lace component 376, the remodeling device 300 is capable of moving between an open configuration and a closed configuration. As illustrated in FIG. 16B, when this embodiment of the remodeling device 300 is in the open configuration, the stent-shaped lace component 376 is extended in length and positioned in somewhat of a collapsed configuration. Further, because of the extended nature of the stent-shaped lace component 376, the proximal end 13 of the first component 12 extends a distance beyond the proximal end 17 of the second component 16. In addition, due to the bias of the at least one spring element 262 extending throughout the remodeling device 300, when the remodeling device 300 is in the open configuration the first and second components 12, 16 are positioned farther apart from one another than when the latch mechanism 380 is fastened.

When the remodeling device 300 of this at least one embodiment is moved into the closed configuration as shown in FIG. 16C, the stent-shaped lace component 376 is moved into an expanded, arch-shaped configuration, thereby defining the interior space 240 and drawing the proximal end 13 of the first component 12 adjacent to the proximal end 17 of the second component 16. In this manner, the latch mechanism 380 of the remodeling device 200 can be engaged, thereby mechanically securing the first and second components 12, 16 together. Furthermore, one of the additional effects of fastening the latch mechanism 380 is that the first and second components 12, 16 are moved closer together and any tissue disposed therebetween is gently compressed.

In general, the remodeling device 300 functions similarly to the application of the remodeling device 200 previously described. However, because the remodeling device 300 comprises two closed ends, the remodeling device 300 is most effectively used in connection with a targeted tissue having a circumference that is capable of being surrounded by the remodeling device 300.

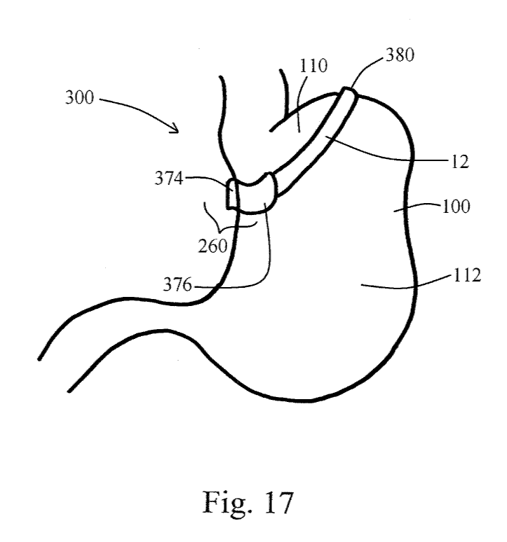

In at least one embodiment, the remodeling device 300 is capable of remodeling an underlying tissue while avoiding constriction and excessive compression thereof. For example and without limitation, in at least one embodiment where the remodeling device 300 is used to treat a stomach 100, the remodeling device 300 can be transversely delivered to the stomach 100 as shown in FIG. 17. Accordingly, the first and second components 12, 16 are positioned adjacent to the anterior and posterior portions of the stomach 100, respectively, and the lace component 260 is disposed proximate to or around the lesser curvature of the stomach 100. To mechanically engage the proximal ends 13, 17 of the first and second components 12, 16, while the second component 16 is held stationary, the first component 12 is pushed forward such that an arched configuration is formed by the stent-shaped lace component 376 as shown in FIG. 16C. In this manner, the proximal end 13 of the first component 12 can be positioned adjacent to the proximal end 17 of the second component 16 and the latch mechanism 380 disposed thereon can be mechanically engaged. Due to the width of the latch mechanism 380 when the components thereof are mechanically engaged and/or the width of the lace component 260 and any pins 22 used in connection with the remodeling device 300, an interior space 70 is formed between the first and second components 12, 16 such that the tissue sandwiched therebetween is not overly compressed.

Similar to the lace component 260 of the remodeling device 200, the arched configuration of the stent-shaped lace component 376 defines the interior space 240. As shown in FIG. 16C, when the remodeling device 300 is applied to the stomach 100 as shown in FIG. 17, the interior space 240 defines an outlet (not shown) from the small gastric pouch 110 so that an outflow of digested matter can flow therethrough and into the lower residual gastric portion 112 of the stomach 100. It will be appreciated that a clinician can modify the dimensions of the lace component 260 and/or the latch mechanism 380 as is necessary to achieve the desired size of the interior space 240 and/or the interior space 70 depending on variables such as the precise treatment for which the remodeling device 300 is to be used and the particular patient.

As described herein, application of the remodeling devices 10, 200, 300 allows a clinician to remodel a targeted tissue while avoiding constriction and excessive compression of the same. Further, the various embodiments described herein allow a clinician to tailor the remodeling device 10, 200, 300 to multiple remodeling applications and various different types of tissues. Permanent remodeling of the tissue is avoided, which prevents adhesions from developing in the underlying targeted tissue and allowed for the complete reversal of the remodeling procedure. Additionally, the remodeling devices 10, 200, 300 are simple to deliver and, as such, the devices 10, 200, 300 may be used in conjunction with other techniques or surgical procedures.

Specifically with respect to the application of the remodeling devices 10, 200, 300 to the stomach 100, using the remodeling devices 10, 200, 300 described herein in the treatment of obesity avoids the nutritional and metabolic deficiencies observed after Malabsorptive Procedures because the normal process of digestion is not altered. In addition, the remodeling devices 10, 200, 300 do not require sutures or staples, which may lead to dehiscence or fistula formation, or produce the degree of regurgitation and vomiting observed in connection with conventional methods used to treat obesity. Moreover, each of the embodiments described herein may be inserted into the body cavity laparoscopically, thereby decreasing the patient's stress associated with the procedure and the patient's recovery time. It will be recognized that any of the devices described herein may be employed in combination with other conventional bariatric procedures.

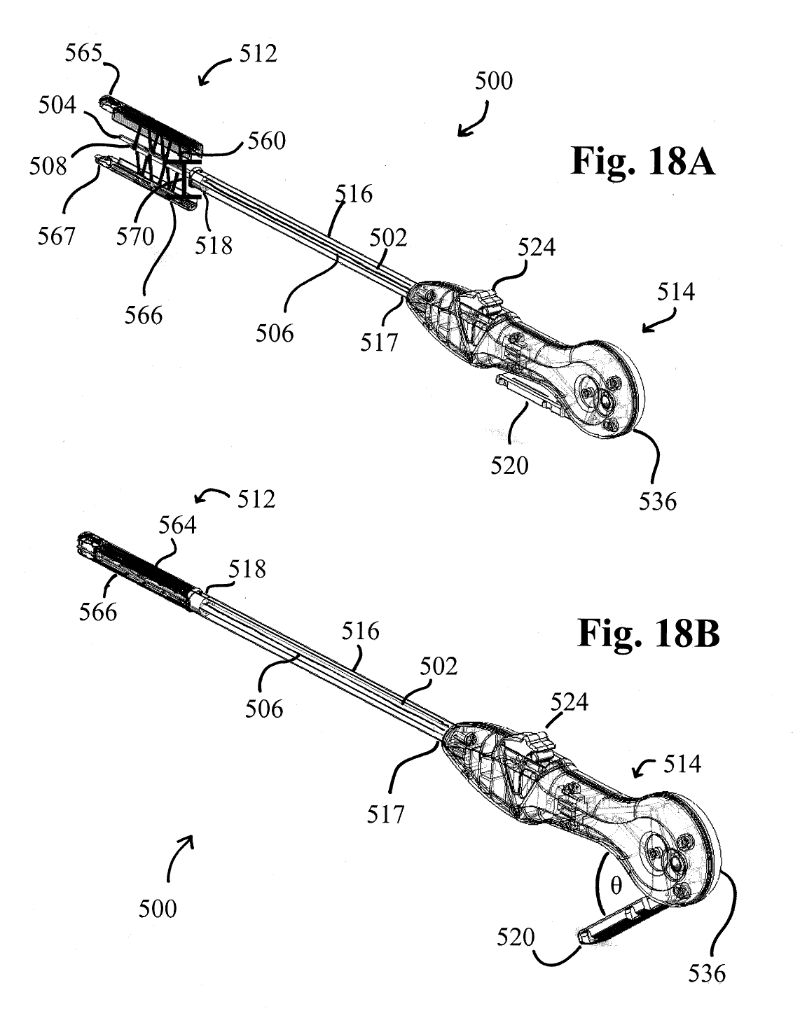

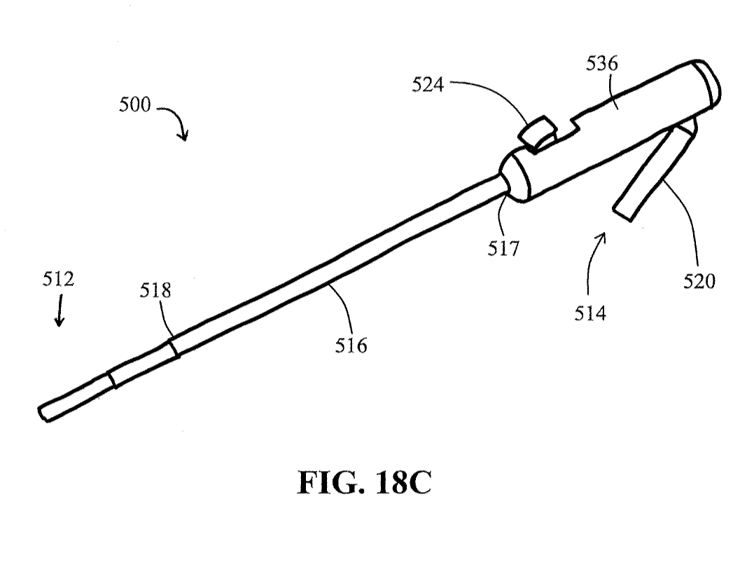

Now referring to FIGS. 18A-18C, at least one embodiment of a delivery device 500 is shown. The delivery device 500 may be used to deliver any of the aforementioned remodeling devices 10, 200, 300 to a tissue or organ of interest laparoscopically and through a parallel closure technique. Specifically, the delivery device 500 utilizes a push-pull mechanism to deliver and deploy a remodeling device 10, 200, 300 to a targeted tissue through a laparoscopic procedure.

FIG. 18A shows a perspective view of at least one embodiment of the delivery device 500. The delivery device 500 comprises a first arm 502, a second arm 506, a lift system 512, and a handle 514. As shown in FIGS. 18A-18C, portions of the first arm 502 and the second arm 506 are slidably disposed within a hollow casing 516 configured for laparoscopic delivery. Furthermore, when the lift system 512 is in the closed configuration (as shown in FIGS. 18B and 18C and described in more detail herein), the lift system 512 may also be slidably disposed within the interior of the hollow casing 516.

The hollow casing 516 comprises a proximal end 517 and a distal end 518. The proximal end 517 of the hollow casing 516 is coupled with the handle 514, while the distal end 518 is open and configured for advancement through the body of a patient. In this manner, the first arm 502, the second arm 506, and the lift system 512 may be disposed within the interior of the hollow casing 516 during laparoscopic delivery and thereafter advanced through the open distal end 518 of the hollow casing 516 to deliver the remodeling device 10, 200, 300 to a targeted tissue.

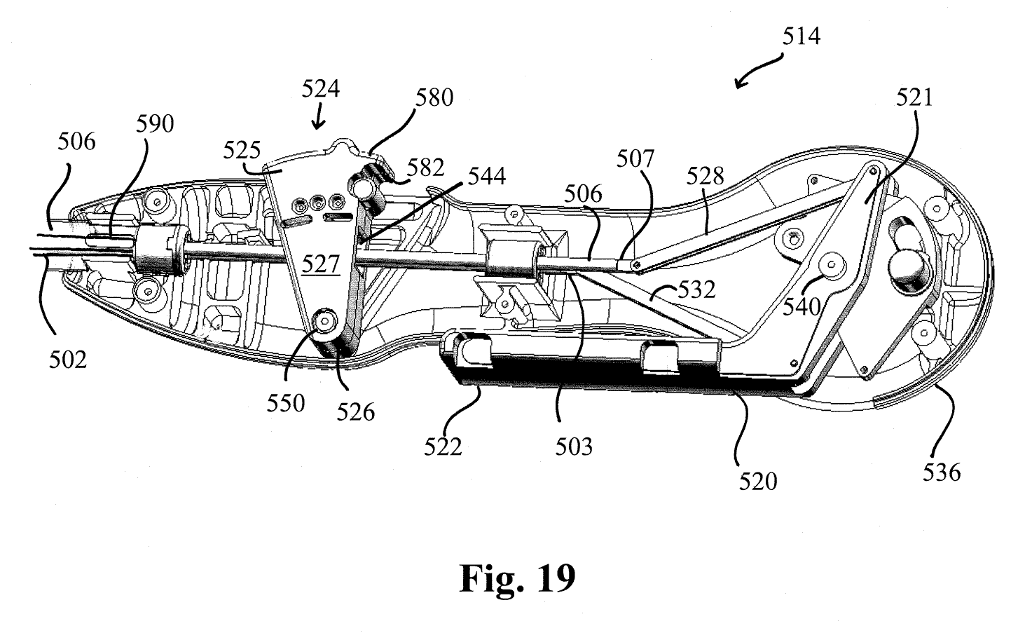

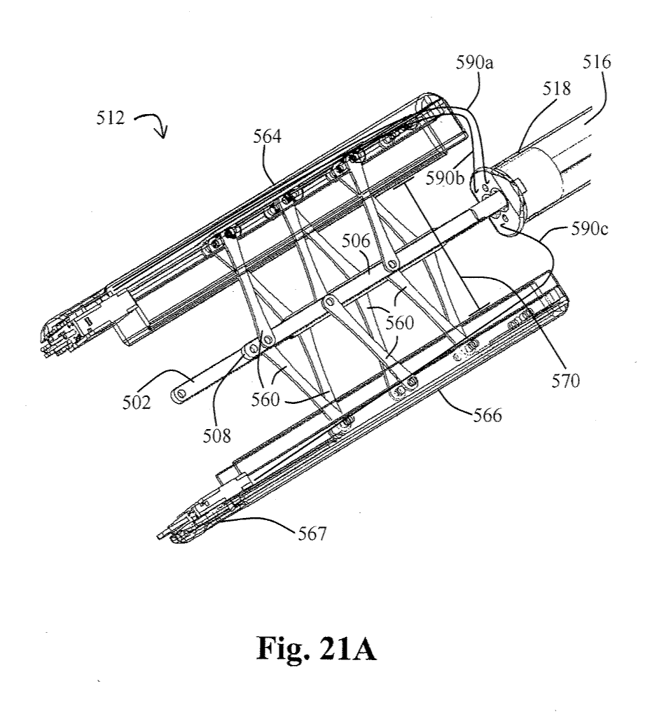

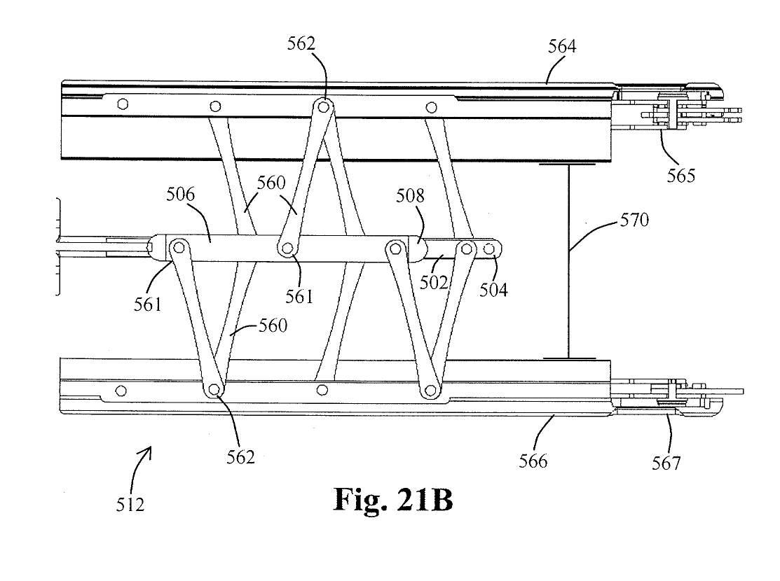

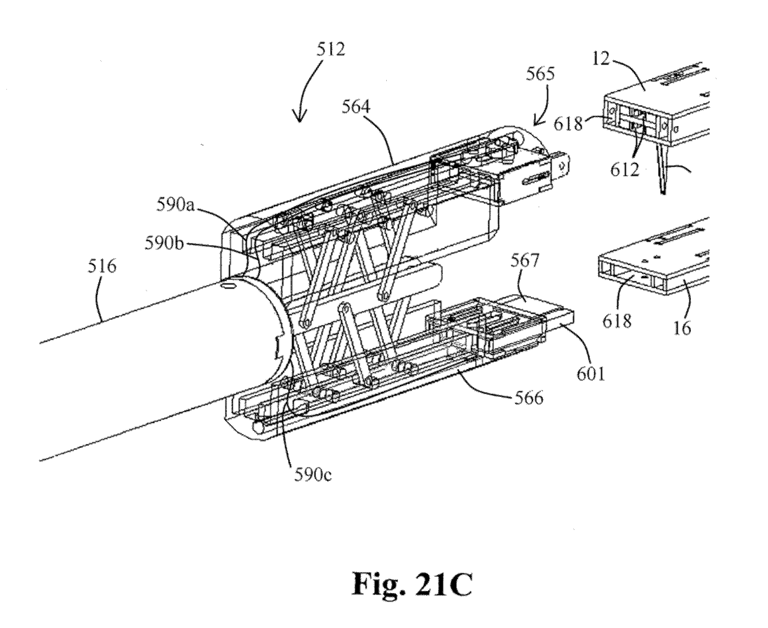

The first and second arms 502, 506 of the delivery device 500 are slidably disposed within the hollow casing 516. In at least one embodiment of the delivery device 500, the first arm 502 has a proximal end 503 and a distal end 504 and the second arm 506 has a proximal end 507 and a distal end 508. The proximal ends 503, 507 of the first and second arms 502, 506 are coupled with the handle 514 (see FIG. 19) and the distal ends 504, 508 of the first and second arms 502, 506 are coupled with the lift system 512 (see FIGS. 21A and 21B). Accordingly, the handle 514 may be manipulated to control the lift system 512 through directing the slidable motion of the first and second arms 502, 506 relative to each other. It will be appreciated that the first arm 502 and the second arm 506 of the delivery device 500 are independent of each other such that a clinician can advance the distal end 504 of the first arm 502 independently of advancing the distal end 508 of the second arm 506 (and vice versa). The movement of the first arm 502 and the second arm 506 relative to each other will be discussed in more detail below.

Now referring to FIG. 19, a cross section of the handle 514 is shown. The handle 514 comprises a first lever 520, a second lever 524, a first shaft 528, a second shaft 532 and a casing 536, and is configured such that a clinician or other user can easily manipulate the first and second levers 520, 524 to facilitate the delivery of the remodeling device 10, 200, 300 to a targeted tissue laparoscopically. The first lever 520 comprises a first end 521 and a second end 522 and may be configured in an angular configuration as is shown in FIG. 19, or in any other configuration that may be suitable to the application. The second end 522 of the first lever 520 extends from the casing 536 and, in at least one embodiment, is configured as a hand grip.

The first end 521 of the first lever 520 is disposed within the casing 536 and is coupled with a hinge 540 such that it is pivotally moveable with respect thereto. For example, the first lever 520 is capable of moving between a collapsed position and an extended position around the hinge 540. In at least one embodiment, when the first lever 520 is in the collapsed position, the second end 522 of the first lever 520 is positioned adjacent to the exterior of the handle 514 as shown in FIGS. 18A and 19. Further, because the first lever 520 is pivotally mounted around the hinge 540, when the first lever 520 is in the collapsed position, the first end 521 of the first lever 520 is positioned proximally relative to the second end 522 of the first lever 520 with respect to the handle 514.

A non-limiting example of the first lever 520 positioned in the extended position is shown in FIG. 18B. In this embodiment, when the first lever 520 is in the extended position, an angle θ is formed between the handle 514 and the second end 522 of the lever 520. As shown in FIG. 18B, the angle θ may comprise an acute angle; however, it will be appreciated that the angle θ may have any value (e.g., 70°, 90°, 100°, 120°, etc.) so long as the second end 522 of the lever 520 is moved some distance away from the handle 514. Furthermore, when the first lever 520 is in the extended position, the first end 521 of the first lever 520 is moved distally within the casing 536 such that it is more closely aligned with the second end 522 of the first lever 520 along the vertical plane of the delivery device 500. In at least one embodiment, the first lever 520 may be spring loaded such that it is biased towards either the extended or the collapsed position when no pressure is applied thereto.

As previously stated, the proximal ends 503, 507 of the first and second arms 502, 506 are coupled with the handle 514. Specifically, in the at least one embodiment shown in FIG. 19, the second arm 506 extends through the interior of the casing 536 and is coupled with the first end 521 of the first lever 520 via the first shaft 528. Similarly, the first arm 502 extends through the interior of the casing 536 adjacent to the second arm 506 and is coupled with the first lever 520 via the second shaft 532.

Because both of the first and second arms 502, 506 are coupled with the first lever 520 via the shafts 528, 532, when the first lever 520 is pivotally moved around the hinge 540, for example between the collapsed and extended positions, the movement is translated to the first and second arms 502, 506 which are slidably moved with respect to each other. Specifically, when the first lever 520 is in the collapsed position, the distal end 504 of the first arm 502 extends beyond the distal end 508 of the second arm 506 (see FIG. 18A). However, when the first lever 520 is moved into the extended position as shown in FIG. 18B, the first shaft 528 advances the second arm 506 and the second shaft 532 retracts the first arm 502 in a concurrent fashion such that the distal end 508 of the second arm 506 extends beyond the distal end 504 of the first arm 502.

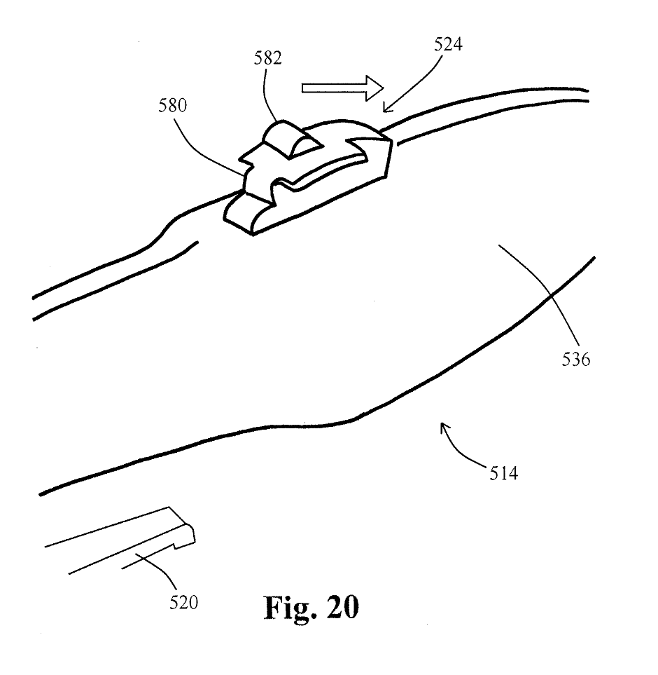

The second lever 524 of the handle 514 comprises a first component 580 and a second component 582 that are independently moveable relative to each other. As is shown in FIGS. 19 and 20, the first and second components 580, 582 may be positioned immediately adjacent to each other; however, it will be appreciated that the two components 580, 582 of the second lever 524 may be positioned in any fashion so long as each of the components 580, 582 is independently moveable relative to the other.

Each of the components 580, 582 of the second lever 524 comprises a first end 525, a second end 526, and a body 527 extending therebetween. The body 527 may be configured in a triangular configuration or in any other configuration that may be suitable. The first end 525 of each of the components 580, 582 of the second lever 524 extends from the casing 536 and may be configured as a thumb pad. The second end 526 of the second lever 524 is disposed within the casing 536 and is coupled with a hinge 550 such that both components 580, 582 of the second lever 524 are pivotally moveable with respect thereto.

The body 527 of each of the components 580, 582 may comprise a channel 544 that is configured to slidably receive the first and/or second arms 502, 506 therethrough. For example, in at least one embodiment, the channel 544 of the first component 580 may be configured to slidably receive the second arm 506 therethrough and the channel 544 of the second component 582 may be configured to slidably receive the first arm 502 therethrough. In at least one alternative embodiment, the channels 544 of the first and second components 580, 582 may each comprise a notch positioned thereon such that the channel 544 on the first component 580 mirrors the channel 544 of the second component 582 (see FIG. 19). Accordingly, due to the proximity and positioning of the first and second components 580, 582, the two channels 544 of the first and second components 580, 582 unite and thereby form one channel 544 through which both the first and second arms 502, 506 extend.

Each of the components 580, 582 of the second lever 524 is further coupled with at least one cable 590. As shown in FIG. 19, the at least one cable 590 may be coupled with a portion of the body 527 of each of the components 580, 582, but it will be understood that the at least one cable 590 may be coupled with any portion of the components 580, 582 of the second lever 524 provided the cable 590 is not attached directly at the hinge 550. In this manner, when one of the components 580, 582 is moved relative to the hinge 590, the relevant component 580, 582 is capable of pulling the cable 590 attached thereto.

Each of the at least one cables 590 of the delivery device 500 extends from the second lever 524 of the handle 514, through the interior of the hollow casing 516 and terminates within either the first or second mounting brackets 564, 566. In the at least one embodiment shown in FIG. 21C, the delivery device 500 comprises a first cable 590a and a second cable 590b, both of which are coupled with the second lever 524 (not shown), extend through the interior of the hollow casing 516 and couple with the distal end of the first mounting bracket 564. Furthermore, a third cable 590c is coupled with the second lever 524 (not shown), extends through the interior of the hollow casing 516 and is coupled with the distal end of the second mounting bracket 566. Details with respect to actuation of the delivery device 500 through the use of the second lever 524 and the cables 590a, 590b, 590c will be described in further detail herein in connection with embodiments of the coupling mechanisms 565, 567 of the first and second mounting brackets 564, 566.

As previously described, the distal ends 504, 508 of the first and second arms 502, 506 are coupled with the lift system 512. The lift system 512 of the delivery device 500 may be any device that is capable of moving the first and second components 12, 16 of the remodeling device 10 relative to each other during the laparoscopic delivery of the remodeling device 10. As shown in FIGS. 21A-21C, in at least one embodiment, the lift system 512 comprises a plurality of shafts 560, a first mounting bracket 564, and a second mounting bracket 568. Each of the plurality of shafts 560 comprises a first end 561 and a second end 562, with the first end 561 hingedly mounted to either the distal end 504 of the first arm 502 or the distal end 508 of the second arm 506 and the second end 562 hingedly mounted to either the first mounting bracket 564 or the second mounting bracket 566. As described in more detail herein, the plurality of shafts 560 enable the lift system 512 to move the mounting brackets 564, 568 (and thereby the components 12, 16 of the remodeling device 10) relative to each other. More specifically, the configuration of the plurality of shafts 560 with respect to the other components of the lift system 512 enables a clinician to move the lift system 512 between an extended position and a collapsed position through the use of the first lever 520 of the handle 514.

The mounting brackets 564, 566 of the lift system 512 are configured to releasably couple with the first and second components 12, 16 of the remodeling device 10, 200, 300. The specific configuration of the mounting brackets 564, 566 enables a clinician to release the proximal ends 13, 17 of the first and second component 12, 16 from the lift system 512 when a release mechanism is triggered through deployment of the second lever 524 of the handle 514. Additionally, either or both of the mounting brackets 564, 566 may further be configured to move the at least on pin 22 of the remodeling device 10, 200, 300 coupled therewith from the substantially retracted position to the substantially extended position when an actuation mechanism is triggered through deployment of the second lever 524 of the handle 514. Accordingly, the delivery device 500 provides a clinician with further control over the placement and delivery of the remodeling device 10, 200, 300 through noninvasive means.

In at least one embodiment, the first mounting bracket 564 comprises a first coupling mechanism 565 disposed on the distal-most end of the first mounting bracket 564. The first coupling mechanism 565 is configured to releasably couple with the proximal end 13 of the first component 12 of the remodeling device 10, 200, 300. Likewise, the second mounting bracket 566 comprises a second coupling mechanism 567 disposed on the distal-most end of the second mounting bracket 566, and is configured to releasably couple with the proximal end 17 of the second component 16 of the remodeling device 10, 200, 300.

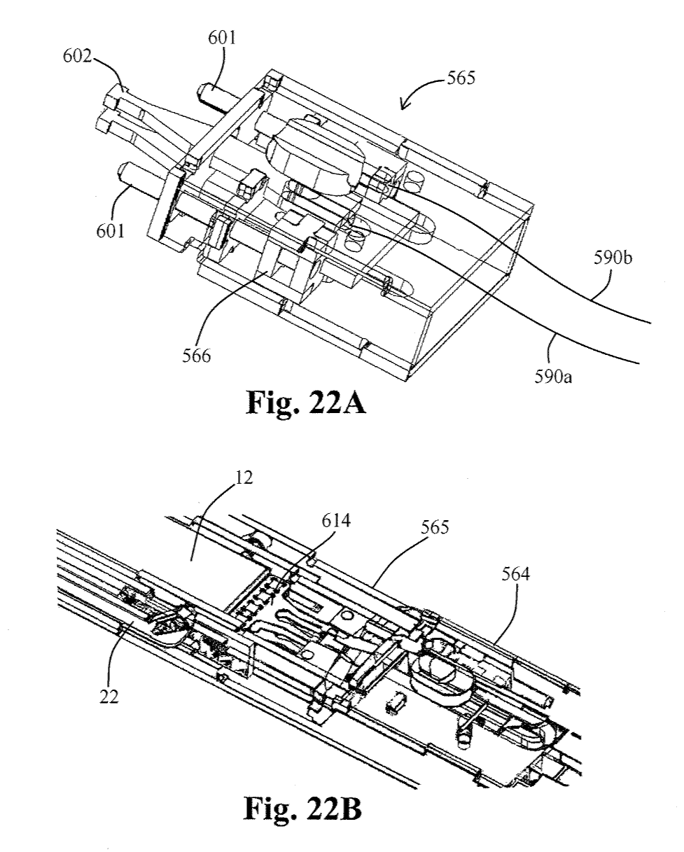

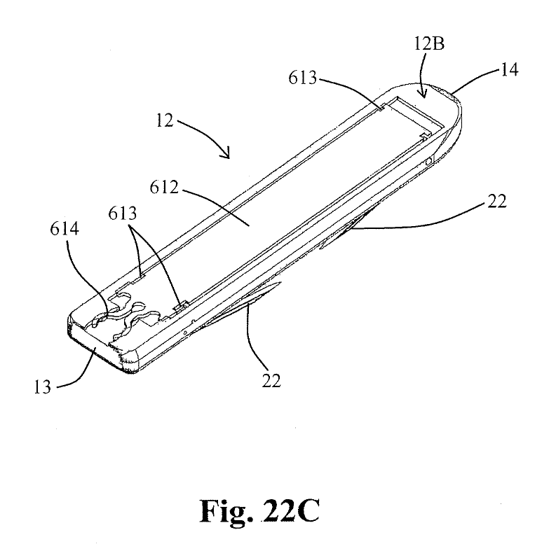

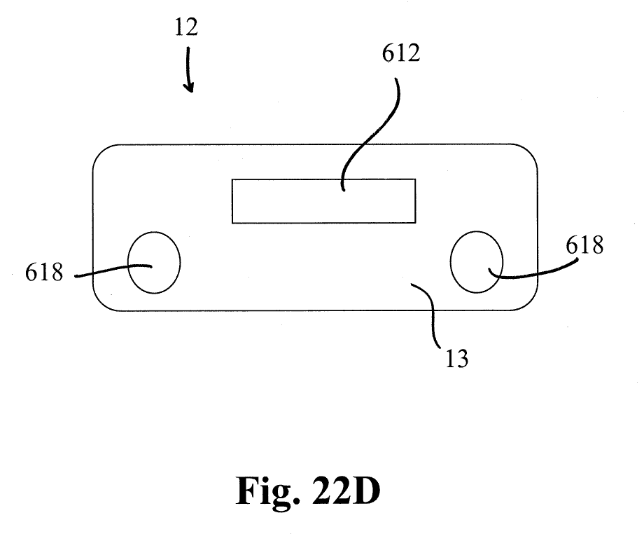

Now referring to FIGS. 22A-22D, several views of the first coupling mechanism 565 are shown. Specifically, FIG. 22A shows a perspective view of at least one embodiment of the first coupling mechanism 565. FIG. 22B shows a perspective view of at least one embodiment of the first coupling mechanism 565 coupled with the proximal end 13 of the first component 12 of the remodeling device 10. FIG. 22C shows a top view of at least one embodiment of the first component 12 configured to be capable of securely, albeit releasably, coupling with the first coupling mechanism 565 of the lift system 512 and facilitating the actuation of the pins 22. Furthermore, FIG. 22D shows a schematic view of the proximal end 13 of the first component 12 of the remodeling device 10 configured to be capable of releasably coupling with the first coupling mechanism of the first mounting bracket 564.

The first coupling mechanism 565 of the first mounting bracket 564 comprises at least one post 601 and a latch mechanism 602. At least portions of both the at least one post 601 and the latch mechanism 602 of the first mounting bracket 564 extend in a cantilevered fashion from the distal end of the mounting bracket 564. In at least one embodiment of the delivery device 500, the latch mechanism 602 of the first coupling mechanism 565 is configured to enable a clinician to move the at least one pin 22 of the component 12, 16 of the remodeling device 10, 200, 300 coupled with the first mounting bracket 564 from the substantially retracted position to the substantially extended position through manipulation of the first component 580 of the second lever 524 of the handle 514. Likewise, the at least one post 601 of the first coupling mechanism 565 is configured to releasably, albeit securely, couple with the proximal end 13, 17 of the component 12, 16 for laparoscopic delivery of the remodeling device to a targeted tissue and thereafter enable a clinician to release the same therefrom upon manipulation of the second component 582 of the second lever 524.

The latch mechanism 602 is coupled with the first component 580 of the second lever 524 via the at least one cable 590 (labeled 590a in FIGS. 21A and 22A for clarification purposes) and may comprise any mechanism that is capable of releasably coupling with the proximal end 13 of the first component 12 of the remodeling device 10, 200, 300. As shown in FIG. 22A, in at least one embodiment, the latch mechanism 602 of the first coupling mechanism 565 may comprise one or more t-shaped pins extending in a cantilevered fashion from the distal end of the first mounting bracket 564. Further, the latch mechanism 602 of the first coupling mechanism 565 is coupled with the at least one cable 590a and additionally may be mounted on a slidable component (not shown) disposed within the distal end of the first mounting bracket 564. In at least one embodiment, the slidable component may comprise a spring-loaded system such that the latch mechanism 602 is biased to remain extended from the distal end of the first mounting bracket 564.

Because the opposite end of the at least one cable 590a is coupled with the first component 580 of the second lever 524 of the handle 514, actuation of the first component 580 of the second lever 524 is translated through the at least one cable 590a and pulls the latch mechanism 602 in a proximal direction along the slidable component. Accordingly, in this at least one embodiment, the latch mechanism 602 extends a prescribed distance from the distal end of the first mounting bracket 564 unless and until the latch mechanism 602 is pulled in a proximal direction by the at least one cable 590a.

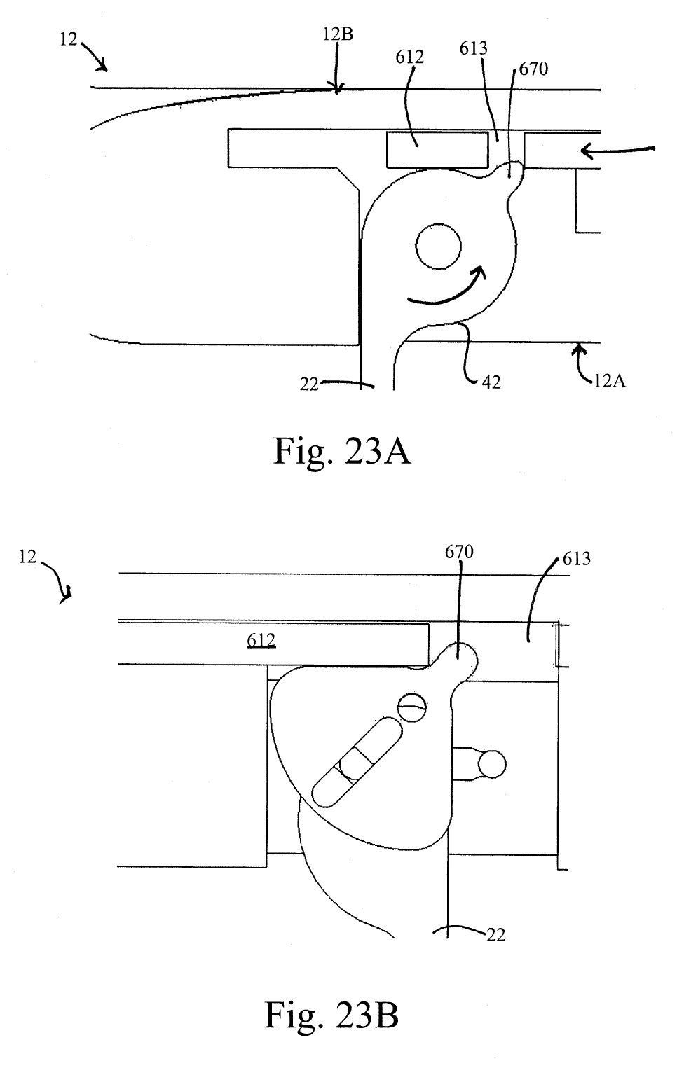

In at least one embodiment, the remodeling device 10, 200, 300 is configured to work in conjunction with the delivery device 500 such that a clinician can control the actuation of the pins 22 of the remodeling device 10, 200, 300 through the second lever 524 of the handle 514. For example, in the at least one embodiment shown in FIG. 22C, the first component 12 further comprises a pin actuation mechanism 612 comprising one or more slots 613 and two prongs 614. In addition, each of the at least one pins 22 of the first component 12 further comprises one or more gear teeth 670 disposed on the proximal end 42 thereof (see FIG. 23). It will be appreciated that while this at least one embodiment is described in connection with the first component 12 of the remodeling device 10, any of the components 12, 16 of the remodeling devices 10, 200, 300 described herein may be similarly configured and used in connection with the first mounting bracket 564 of the delivery device 500.

As shown in FIG. 22C, the pin actuation mechanism 612 comprises a slidable plate embedded in or disposed on the second side 12B of the first component. The one or more slots 613 of the pin actuation mechanism 612 are formed along the edges of the plate and disposed in accordance with the positioning of the at least one pin 22 of the first component 12 of the remodeling device 10. Accordingly, as shown in FIG. 22C, the one or more slots 613 are positioned proximate to each of the at least one pins 22 of the first component 12 and configured such that the gear teeth 670 of the respective pin 22 can extend therethrough (see FIGS. 23A and 23B).

The slidable plate of the pin actuation mechanism 612 is capable of sliding along the second side 12B of the first component 12 in a defined path. As such, the slidable plate is prevented from traversing the proximal or distal ends 13, 14 of the first component 12 by way of ridges, obstructions, or other means extending from the second side 12B of the first component 12. When the pin actuation mechanism 612 is caused to slide along the second side 12B of the first component 12, due to the placement of the slots 613 relative to the gear teeth 670 of the pins 22, the sliding of the plate causes the slots 613 to move, which thereby causes the pins 22 to move between the substantially retracted position and the substantially extended position. FIGS. 23A and 23B show cross-sectional views of the pins 22 of the first component 12 and their movement relative to the movement of the pin actuation mechanism 612 along the second side 12B of the first component 12.

Referring back to FIGS. 22A and 22B, the two prongs 614 of the pin actuation mechanism 612 extend from the proximal-most end of the pin activation mechanism 612 and are configured to releasably, albeit securely, receive the latch mechanism 602 of the first coupling mechanism 565. At least one specific embodiment of the two prongs 614 is shown in FIGS. 22A and 22B. Here, the two prongs 614 comprise a hairpin configuration such that a space is formed therebetween. Accordingly, the two prongs 614 are capable of securely retaining the t-shaped end of the at least one pin of the latch mechanism 602 of the delivery device 500 after the at least one pin is positioned therein. In addition, due to the configuration, the two prongs 614 are further capable of releasing the latch mechanism 602 when a sufficient amount of proximal force is applied to the interior surfaces of the two prongs 614.

When a sufficient amount of pressure is applied to the interior surfaces of one or both of the prongs 614 of the pin actuation mechanism 612, the two prongs 614 are capable of releasing the portion of the latch mechanism 602 retained therein. In at least one embodiment, the two prongs 614 are configured such that when sufficient pressure is applied to the interior of the prongs 614, the mating mechanism 612 widens and the space between the two prongs 614 is increased. Accordingly, while the two prongs 614 are biased to remain a prescribed distance apart and thus exhibit a prescribed width, when a sufficient force is applied to the interior surfaces of the two prongs 614, the space between the two prongs 614 increases, thereby releasing any component contained therein.

Due to the configuration of the delivery device 500, a clinician can easily manipulate the first component 580 of the second lever 524 of the handle 514 to move the pins 22 of the remodeling device 10 from the substantially retracted position to the substantially extended even during a complete laparoscopic delivery of the remodeling device 10. For example and without limitation, when the first component 580 of the second lever 524 is manipulated, the cable 590a coupled therewith is pulled in a proximal direction (i.e. toward the handle 514 of the delivery device 500). As the latch mechanism 602 is mounted on a sliding component (not shown) within the first mounting bracket 564, the force provided through the cable 590a moves the latch mechanism 602 in a proximal direction along the sliding component. In this manner, the t-shaped pin seated within the two prongs 614 of the pin actuation mechanism 612 is withdrawn toward and/or into the interior of the first mounting bracket 564, which causes the pin actuation mechanism 612 to slide along the second side 12B of the first component 12 toward the proximal end 13 thereof. In this manner, the gear teeth 670 of the pins 22 are moved in accordance with the slots 613 through which they extend, thereby causing the pins 22 of the remodeling device 10 to rotate from the substantially retracted position to the substantially extended position.

When the slidable plate of the pin actuation mechanism 612 is prevented from further proximal movement due to the ridges, obstructions and/or other means extending from the second side 12B of the first component 12, the pressure exerted on the interior portion of the prongs 614 by way of the latch mechanism 602 seated therein via the cable 590a is sufficiently increased such that the two prongs 614 of the pin actuation mechanism 612 open and release the t-shaped pin(s) of the latch mechanism 602. In this manner, a clinician can deploy the pins 22 of the remodeling device 10, 200, 300 simply through manipulation of the first component 580 of the second lever 524 of the handle 514, thereby allowing for the delivery procedure to be entirely laparoscopic in nature.

It is contemplated that any number of configurations can be used in connection with the latch mechanism 602 and the pin actuation mechanism 612. For example and without limitation, at least one alternative configuration of the pin actuation mechanism 612 of the first component 12 of the remodeling device 10, 200, 300 is shown in FIG. 22C. This at least one embodiment of the pin actuation mechanism 612 is adapted to receive a latch mechanism 602 comprising a rounded configuration as opposed to the t-shaped configuration of the at least one pin shown in FIGS. 22A and 22B.

It will be appreciated that while specific embodiments of the latch mechanism 602 and the proximal end 13 of the first component 12 of the remodeling device 10 have been described and illustrated herein, the latch mechanism 602 may comprise any mechanism that is capable of moving the pins 22 of the remodeling device 10 from the substantially retracted position to the substantially extended position through operation of the handle 514. Furthermore, the configuration of the proximal ends 13, 17 of the first and second components 12, 16 of the remodeling device 10, 200, 300 may be constructed in any fashion capable of releasably mating with the latch mechanism 602 of the delivery device 500, and it will be appreciated that the latch mechanism 602 and the related mating mechanism 612 may be used in connection with any of the first and second mounting brackets 564, 566 and/or the first and second components 12, 16 of the remodeling device 10, 200, 300.

As previously described, the first coupling mechanism 565 further comprises at least one post 601 extending from the distal end of the first mounting bracket 564 in a cantilevered fashion. The posts 601 of the first coupling mechanism 565 enable a clinician to releasably couple the a component 12, 16 of the remodeling device 10, 200, 300 with the delivery device 500 and thereafter disengage the component 12, 16 from the first mounting bracket 564 through manipulation of the second component 582 of the second lever 524 of the handle 514. In this manner, a clinician can easily release the first or second component 12, 16 when the remodeling device 10, 200, 300 is properly positioned on a targeted tissue. Accordingly, the at least one post 601 is coupled with the second component 582 of the second lever 524 via the at least one cable 590 (labeled 590b in FIGS. 21A and 22A for clarification purposes).

The at least one post 601 of the first coupling mechanism 565 may comprise any configuration, provided it is capable of releasably coupling with the proximal end 13 of a first component 12 of the remodeling device 10, 200, 300. By way of example and without limitation, as shown in FIG. 22A, the first coupling mechanism 565 may comprise two cylindrical posts 601 disposed on lateral portions of the first coupling mechanism 565 with the latch mechanism 602 positioned therebetween. Each of the at least one posts 601 of the first coupling mechanism 565 is coupled with the cable 590b and is mounted on a slidable component (not shown) disposed within the distal end of the first mounting bracket 564, which, in at least one embodiment, may comprise a spring-loaded system. Further, in at least one embodiment, each of the at least one posts 601 may comprise a ferromagnetic component for promoting the releasable engagement between the first coupling mechanism 565 and the component 12, 16 of the remodeling device 10, 200, 300.

Because the least one cable 590b is additionally coupled with the second component 582 of the second lever 524 of the handle 514, actuation of the second component 582 of the second lever 524 is translated through the at least one cable 590b and thus pulls the at least one post 601 in a proximal direction along the slidable component. Accordingly, in this at least one embodiment, the at least one post 601 extends a prescribed distance from the distal end of the first mounting bracket 564 unless and until the at least one post 601 is pulled in a proximal direction by the at least one cable 590b.

As previously described, a component 12, 16 of the remodeling device 10, 200, 300 may be securely, albeit releasably, coupled with the first mounting bracket 564 by way of the at least one post 601 by slidably inserting the cantilevered portion(s) of the at least one post 601 of the first coupling mechanism 565 into the proximal end 13, 17 of the applicable component 12, 16 of the remodeling device 10, 200, 300. Thereafter, a clinician can release the first and/or second components 12, 16 of the remodeling device 10, 200, 300 from the delivery device 500 through manipulation of the second component 582 of the second lever 524 of the handle 514.

Specifically, as shown in FIG. 22D, in at least one embodiment, the proximal end 13 of the first component 12 of the remodeling device 10 may comprise at least one receptacle 618 disposed thereon that is configured to receive the at least one post 601 of the first coupling mechanism 565. Furthermore, the at least one receptacle 618 may comprise a means for retaining the at least one post 601 therein until a sufficient amount of proximal force is applied to withdraw the at least one post 601 from the receptacle 618.

While FIG. 22D illustrates an embodiment comprising two receptacles 618 having a circular configuration and disposed on the lateral portions of the component 12, 16, it will be appreciated that the receptacle(s) 618 may be positioned on any portion of the relevant component 12, 16 of the remodeling device 10, 200, 300 so long as the receptacle(s) 618 can receive the at least one post 601 when the first mounting bracket 564 is coupled with the proximal end 13, 17 of the component 12, 16. It will further be appreciated that the receptacle 618 may also comprise any configuration provided, however, that the at least one post 601 is capable of slidably mating with the corresponding receptacle 618 when the first component 12 is coupled with the first mounting bracket 564.

Due to the configuration of the delivery device 500 and the first component 12, a clinician can easily manipulate the second component 582 of the second lever 524 of the handle 514 to release the at least one post 601 from the applicable receptacle 618, and thus the first component 12 of the remodeling device 10, during a laparoscopic procedure. For example and without limitation, when the second component 582 of the second lever 524 is manipulated, the cable 590b coupled therewith is pulled in a proximal direction (i.e. toward the handle 514 of the delivery device 500). Because the posts 601 are mounted on a sliding component (not shown) within the first mounting bracket 564, the force provided through the cable 590b moves the posts 601 in a proximal direction along the sliding component. In this manner, the posts 601 are withdrawn from the receptacles 618, which causes the first component 12 of the remodeling device 10 to release from the delivery device 500 (provided the latch mechanism 602 has been previously uncoupled from the pin actuation mechanism 612). In this manner, a clinician can easily release the first component 12 from the delivery device 500 simply through manipulation of the first component 580 of the second lever 524 of the handle 514, thereby allowing for the delivery procedure to be entirely laparoscopic in nature.

It will be appreciated that while specific embodiments of the at least one post 601 and the proximal end 13 of the first component 12 of the remodeling device 10, 200, 300 have been described and illustrated herein, the at least one post 601 may comprise any releasable mechanism that is capable of securely, albeit releasably, coupling with a component 12, 16 of the remodeling device 10, 200, 300 and thereafter releasing the same therefrom through operation of the handle 514. Furthermore, the configuration of the proximal ends 13, 17 of the first and second components 12, 16 of the remodeling device 10, 200, 300 may be constructed in any fashion capable of releasably mating with the at least one post 601 of the delivery device 500, and it will be appreciated that the at least one post 601 and related receptacle(s) 618 may be used in connection with any of the first and second mounting brackets 564, 566 and/or the first and second components 12, 16 of the remodeling device 10, 200, 300.

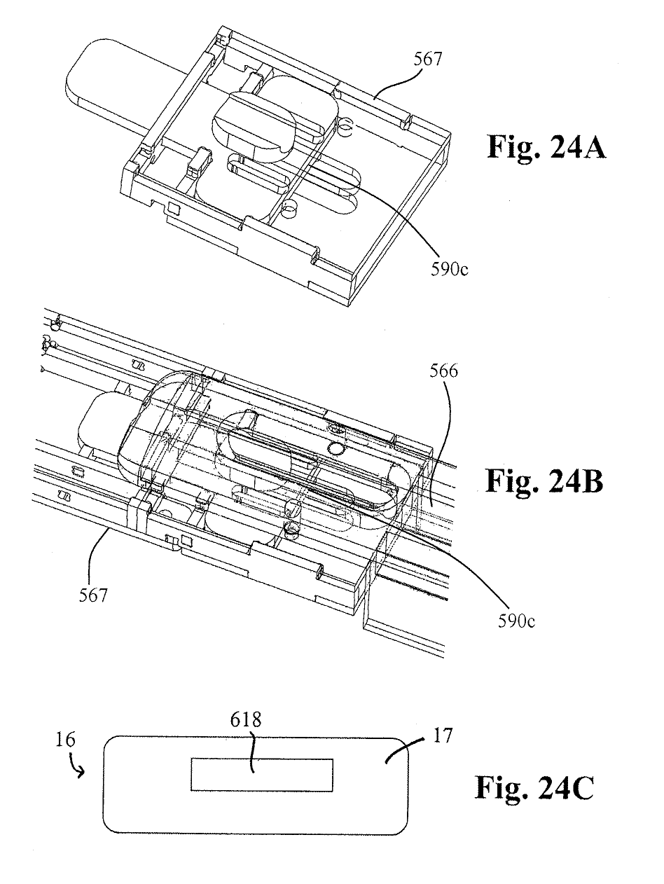

As previously described, the second mounting bracket 566 comprises a second coupling mechanism 567. Now referring to FIGS. 24A-24C, perspective views are shown of the second coupling mechanism 567. In this at least one embodiment, the proximal end 17 of the second component 16 of the remodeling device 10 is configured to releasably engage with the second coupling mechanism 567 such that the second component 16 is securely coupled therewith (see FIG. 24B). Further, the second coupling mechanism 567 is configured to release the proximal end 17 of the second component 16 when the release mechanism is triggered through deployment of the second component 582 of the second lever 524 of the handle 514.

In at least one embodiment and similar to the first mounting bracket 564, the second mounting bracket 566 further comprises a second coupling mechanism 567 having at least one post 601 extending from the distal end thereof in a cantilevered fashion. The at least one post 601 is configured to be slidably received within the proximal end 17 of the second component 16 of the remodeling device 10 and is further coupled with the second component 582 of the second lever 524 via the at least one cable 590 (labeled 590c in FIGS. 21A and 24A for clarification purposes). Further, in at least one embodiment, each of the at least one posts 601 of the second mounting bracket 566 may comprise a ferromagnetic component for promoting the releasable engagement between the second coupling mechanism 567 and the component 12, 16 of the remodeling device 10, 200, 300.

The at least one post 601 of the second coupling mechanism 567 may comprise any configuration, provided it is capable of releasably coupling with the proximal end 17 of a second component 16 of the remodeling device 10. By way of example and without limitation, as shown in FIG. 24A, the second coupling mechanism 567 may comprise a single post 601 configured in a plate-like configuration and extending from the center portion of the distal-most end of the second mounting bracket 566. Alternatively, the at least one post 601 of the second mounting bracket 566 may be comprised similarly to the at least one posts of the first mounting bracket 564 previously described herein.

Irrespective of the number and/or configuration of the at least one posts 601 of the second coupling mechanism 567, each of the at least one posts 601 of the second coupling mechanism 567 is coupled with the cable 590c and is mounted on a slidable component (not shown) disposed within the distal end of the second mounting bracket 566, which, in at least one embodiment, may comprise a spring-loaded system. Because the least one cable 590c is additionally coupled with the second component 582 of the second lever 524 of the handle 514, actuation of the second component 582 of the second lever 524 is translated through the at least one cable 590c and pulls the at least one post 601 of the second coupling mechanism 567 in a proximal direction along the slidable component. Accordingly, in this at least one embodiment, the at least one post 601 extends a prescribed distance from the distal end of the second mounting bracket 566 unless and until the at least one post 601 is pulled in a proximal direction by the at least one cable 590c.

As previously described in connection with the first mounting bracket 564, a clinician can release the first and/or second components 12, 16 of the remodeling device 10, 200, 300 from the delivery device 500 through manipulation of the second component 582 of the second lever 524 of the handle 514. Specifically, with respect to the second mounting bracket 566, a component 12, 16 of the remodeling device 10, 200, 300 may be securely, albeit releasably, coupled therewith by way of the slidable insertion of the cantilevered portion(s) of the at least one post 601 into the proximal end 13, 17 of the applicable component 12, 16 of the remodeling device 10, 200, 300.

As shown in FIG. 24C, in at least one embodiment, the proximal end 17 of the second component 16 of the remodeling device 10 may comprise at least one receptacle 618 disposed thereon that is configured to receive the at least one post 601 of the second coupling mechanism 566. Furthermore, similar to the receptacles 618 described in connection with the first coupling mechanism 564, the at least one receptacle 618 of the second coupling mechanism 567 may comprise a means for retaining the at least one post 601 therein until a sufficient amount of proximal force is applied to withdraw the at least one post 601 from the receptacle 618.

Due to the configuration of the delivery device 500 and the second component 17, a clinician can easily manipulate the second component 582 of the second lever 524 of the handle 514 to release the at least one post 601 from the applicable receptacle 618, and thus the second component 17 of the remodeling device 10, during a laparoscopic procedure.

For example and without limitation, when the second component 582 of the second lever 524 is manipulated, the cable 590c coupled therewith is pulled in a proximal direction (i.e. toward the handle 514 of the delivery device 500). Because the posts 601 are mounted on a sliding component (not shown) within the second mounting bracket 566, the force provided through the cable 590c moves the at least one post 601 in a proximal direction along the sliding component. In this manner, the post 601 is withdrawn from the receptacle 618 on the second component 16, which causes the second component 16 of the remodeling device 10 to release from the delivery device 500. Accordingly, through use of the second lever 524 of the handle 514, a clinician can easily release the second component 16 from the delivery device 500, thereby allowing for the delivery procedure to be entirely laparoscopic in nature. Furthermore, it should be noted that in at least one embodiment, activation of the release mechanism through manipulation of the second component 582 of the second lever 524 of the handle 514 is not only limited to the release of the second component 16 from the second mounting bracket 566. Rather, because both cables 590b and 590c may be coupled with the second lever 524, activation of the second component 524 can simultaneously cause the cables 590b and 590c to pull in a proximal direction, thereby causing the posts 601 of both the first and second coupling mechanisms 565, 567 to simultaneously withdraw from the components 12, 16 and releasing the same from the delivery device 500.

It will be appreciated that while specific embodiments of the at least one post 601 and the proximal end 17 of the second component 16 of the remodeling device 10 have been described and illustrated herein, the at least one post 601 may comprise any releasable mechanism that is capable of securely, albeit releasably, coupling with a component 12, 16 of the remodeling device 10, 200, 300 and thereafter releasing the same therefrom through operation of the handle 514. Furthermore, the configuration of the proximal ends 13, 17 of the first and second components 12, 16 of the remodeling device 10, 200, 300 may be comprised in any fashion capable of releasably mating with the at least one post 601 of the second coupling mechanism 567 of the delivery device 500, and it will be appreciated that the at least one post 601 and related receptacle(s) 618 may be used in connection with any of the first and second mounting brackets 564, 566 and/or the first and second components 12, 16 of the remodeling device 10, 200, 300.

Now referring back to FIGS. 18A-18C and 21A-21C, certain capabilities of the lift system 512 to move the first and second mounting brackets 564, 566 (and thus the components 12, 16 of the remodeling device 10 coupled therewith) will be described. In the at least one embodiment shown in FIGS. 18A and 21A, each of the shafts 560 is coupled with either the first or second arms 502, 506 and the first or second mounting brackets 564, 566 in such a manner that when the distal end 504 of the first arm 502 is extended beyond the distal end 507 of the second arm 504 the plurality of shafts 560 are in a substantially extended configuration (see FIGS. 18A, 21A and 21B), and when the distal end 507 of the second arm 506 is advanced beyond the distal end 504 of the first arm 502 through operation of the first lever 520, the plurality of shafts 560 are moved into a collapsed configuration (see FIGS. 18B and 18C). Specifically, when the lift system 512 is in the expanded configuration, the mounting brackets 564, 566 are held a distance 570 apart by the plurality of shafts 560. For example, the distance 570 may be greater than 3.5 centimeters and, in at least one embodiment, the distance 570 may comprise between about 3.8 and about 4 centimeters. Alternatively, when the lift system 512 is in the collapsed configuration, each shaft 560 is substantially parallel with the first and second arms 502, 506 and forms a folded configuration. Accordingly, in the collapsed configuration, the distance 570 between the mounting brackets 564, 566 may be negligible. In at least one embodiment, when the lift system 512 is in the collapsed configuration, the total diameter of the lift system 512 and any components 12, 16 of the remodeling device 10, 200, 300 coupled therewith is less than about 1.5 centimeters. In this manner, the delivery device 500 and the remodeling device 10 are capable of being inserted into the abdominal cavity of a patient through a 1.5 centimeter trocar.

The movement of the lift system 512 from the extended configuration to the collapsed configuration decreases the distance 570 between the first and second mounting brackets 564, 566 and simultaneously moves the mounting brackets 564, 566 toward each other. Accordingly, when the delivery device 500 is coupled with the first and second components 12, 16 of the remodeling device 10, the movement of the lift system 512 may used to achieve the parallel closure of the first and second components 12, 16 of the remodeling device 10 around a tissue of interest. Due to this parallel closure technique, the at least one pin 22 of the remodeling device 10 positioned in the substantially extended configuration is driven through the targeted tissue not only by the force of the opposing component against which the distal end 44 of the at least one pin 22 will eventually mechanically engage, but also by the force of the component to which the proximal end 42 of the at least one pin 22 is attached. In this manner, the delivery device 500 facilitates a safe and effective delivery technique of the remodeling device 10, 200, 300 to the desired location on the targeted organ or tissue.

For the sake of simplicity, while the delivery device 500 is described herein in connection with delivering the remodeling device 10, it will be appreciated that the delivery device 500 may also be utilized to laparoscopically deliver the remodeling device 200 and/or the remodeling device 300 to a targeted tissue. Accordingly, except where expressly stated, any reference herein with respect to use of the delivery device 500 in connection with the remodeling device 10 will be considered to also be applicable to use of the delivery device 500 in connection with the remodeling device 200 and/or the remodeling device 300.

In operation, the remodeling device 10 is coupled with the first and second mounting brackets 564, 566 as previously described herein and the lift system 512 is moved into the collapsed position. In at least one embodiment, the lift system 512 and/or the remodeling device 10 coupled therewith may be withdrawn into the interior of the hollow casing 516 in preparation for laparoscopic delivery. Alternatively, the remodeling device 10 and the lift system 512 may be positioned distally of the distal end 518 of the hollow casing 516.

While the lift system 512 may be advanced laparoscopically into the patient's body in the collapsed position, it will be appreciated that the lift system 512 may be positioned somewhere between the substantially collapsed and substantially extended positions so long as the lift system 512 is capable of being easily advanced through an abdominal port and/or trocar. When the delivery device 500 and the remodeling device 10 are in the desired position, the distal end of the delivery device 500 is advanced, under fluoroscopic guidance or otherwise, through the abdominal cavity and to a location adjacent to the targeted tissue.

Thereafter, the first and second components 12, 16 of the remodeling device 10 are maneuvered out of the hollow casing 516 through the simultaneous advancement of the first and second arms 202, 206 (if the same are not already positioned accordingly). The clinician can then manipulate the lift system 512 by way of the first and second levers 520, 524 of the handle 514 to maneuver the first and second components 12, 16 relative to each other and the targeted tissue such that the remodeling device 10 can be accurately delivered to the tissue of interest. For example, as previously described in connection with the specific components of the delivery device 500, a clinician can move the lift system 512 between the substantially collapsed and substantially extended positions through manipulation of the first lever 520, actuate the pins 22 of the first component 12 of the remodeling device 10 through manipulation of the first component 580 of the second lever 524, and/or release the first and second components 12, 16 from the lift system 512 altogether through manipulation of the second component 582 of the second lever 524.

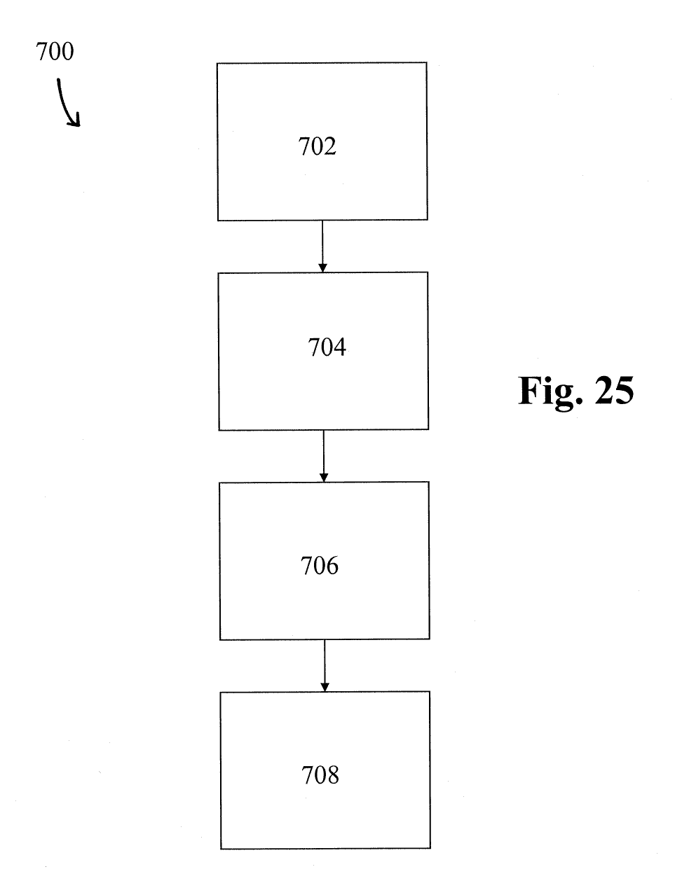

Now referring to FIG. 25, a flow chart of a method 700 for laparoscopically delivering the remodeling device 10 is shown. For ease of understanding, the steps of the related methods described herein will be discussed relative to the components of the remodeling device 10 and, at least in part, the delivery device 500, but it will be appreciated that any device can be used to perform these methods so long as the device is capable of achieving a secure mechanical engagement through a piece of tissue without overly compressing the tissue disposed therebetween. Furthermore, while the methods described herein are described in connection with embodiments of the remodeling device 10, 200, 300, and/or the delivery device 500, it will be appreciated that various additional devices may be used to facilitate the laparoscopic delivery of the remodeling device 10, 200, 300 such as a camera, light and/or a device for delivering a gas to a targeted area.

At step 702, the first and second components 12, 16 are advanced laparoscopically into the patient's body. In at least one embodiment, the remodeling device 10 may be inserted through a 15 millimeter cannula under insufflation into the appropriate cavity of the patient's body. Under fluoroscopic, direct camera control or otherwise, at step 704, the first side 12A of the first component 12 is positioned adjacent to the desired surface of a targeted tissue. Accordingly, in an embodiment where the first side 12A of the first component 12 comprises one or more pins 22, the distal end 44 of each of the pins 22 is positioned proximate to the desired surface of the targeted tissue. As such, in those embodiments of the remodeling device 10, 200, 300 where the at least one pin 22 is moveable between the collapsed and extended configurations, the at least one pin 22 is moved into the extended configuration at step 704.

At step 706, also under fluoroscopic, direct camera control or otherwise, the first side 16A of the second component 16 is positioned on an opposite side of the targeted tissue such that the desired affect may be achieved when the first and second 12, 16 are mechanically engaged with each other. It will be appreciated that steps 704 and 706 may occur simultaneously or subsequently, depending upon the preference of the clinician and the specifics of the patient and the targeted tissue.

Thereafter, the first and second components 12, 16 are mechanically engaged through the targeted tissue at step 708 such that the targeted tissue is sandwiched and secured therebetween. In the embodiments of the remodeling device 10, 200, 300 where either or both of the first sides 12A, 16A of the first and second components 12, 16 comprise one or more of pins 22 extending therefrom, the first sides 12A, 16A of the two components 12, 16 are simultaneously moved toward each other—either by the application of pressure to the second sides 12B, 16B or otherwise—thereby causing the one or more pins 22 to pierce the underlying targeted tissue and register into the engagement mechanism 28 disposed in the first side 12A, 16A of the opposite component 12, 16. In this manner, the at least one pin 22 of the remodeling device 10, 200, 300 takes up the majority of the compressional load exerted on the underlying tissue by the remodeling device 10 and prevents the over-compression of the same.

Alternatively, the method 700 may be slightly modified to accommodate certain embodiments of the remodeling device 300. Specifically, in at least one embodiment of the method 700, at step 708 the two components 12, 16 may simultaneously be moved toward each other and, concurrently or consecutively, the first component 12 may be moved in a distal direction relative to the second component 16 such that the components of the latch mechanism 380 are aligned and the stent-shaped lace component 376 is moved into the arched configuration. After the proximal ends 13, 17 of the first and second components 12, 16 are aligned, the latch mechanism 380 is secured through use of any laparoscopic device known in the art that may be inserted into the abdominal cavity through a separate port. For example and without limitation, a pair of laparoscopic graspers may be employed to facilitate the distal movement of the first component and/or to secure the latch mechanism 380.

After the latch mechanism 380 is secured, the remodeling device 300 compresses the underlying tissue between the first and second components 12, 16, yet bears the majority of the compressional load due to the defined interior 70. Accordingly, in each of the embodiments of the method 700 described herein, the remodeling device 10, 200, 300 is capable of effectively remodeling and/or providing support to the underlying tissue or organ in a desired manner.

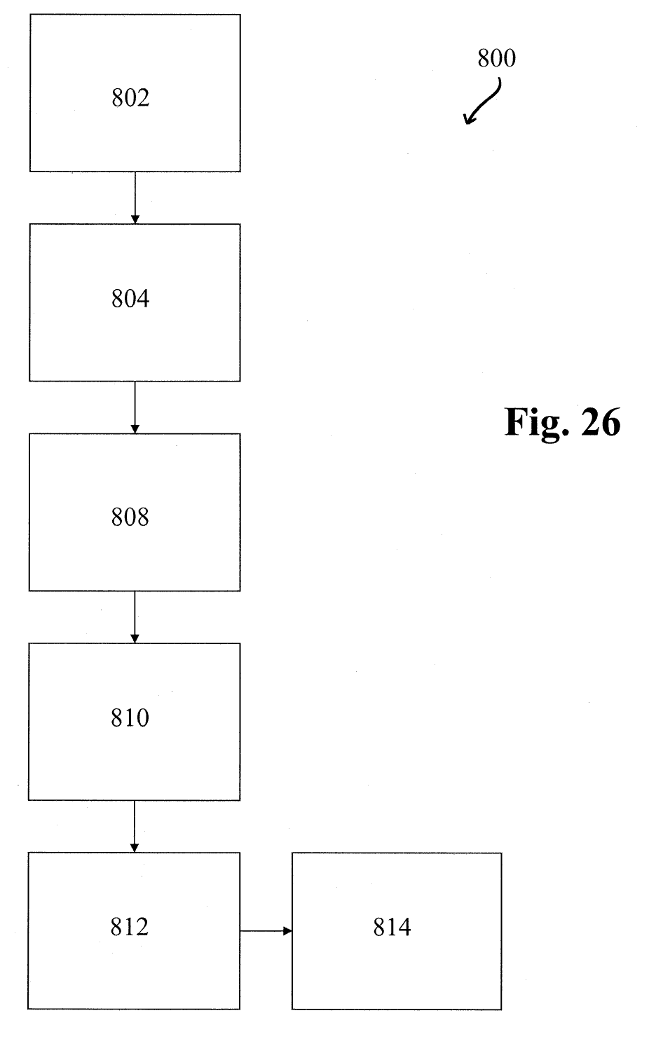

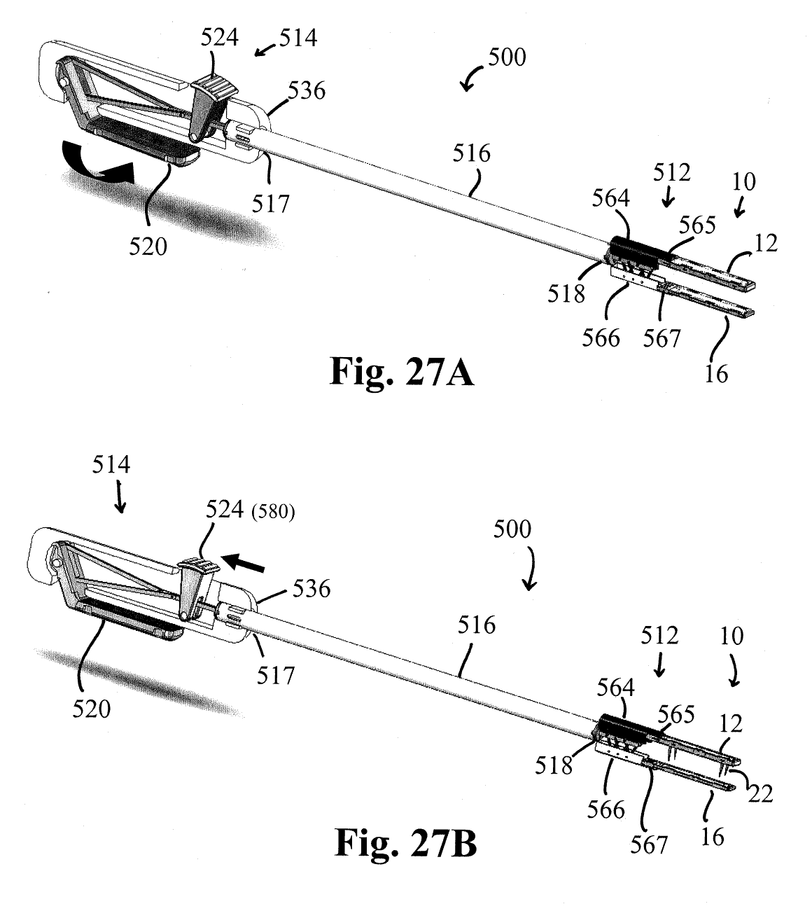

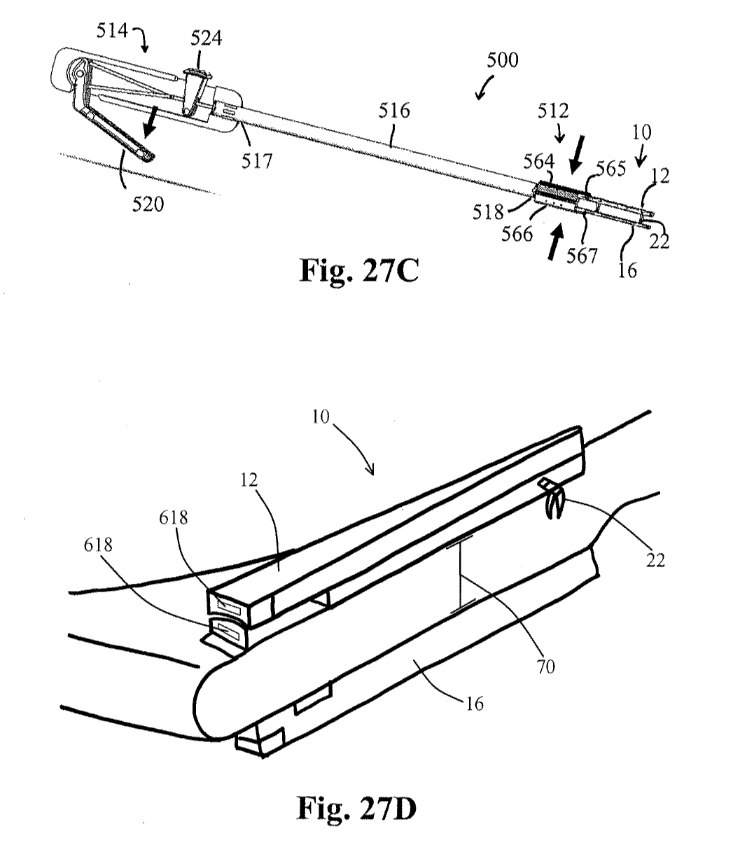

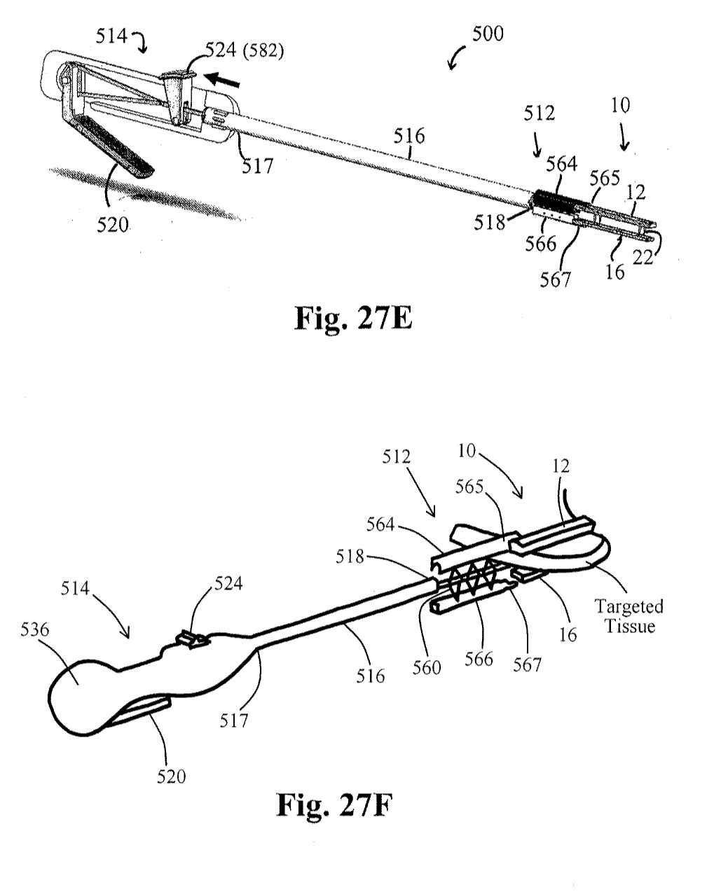

Now referring to FIG. 26, a flow chart of a method 800 for laparoscopically delivering the remodeling device 10 through the use of the delivery device 500 is shown. Solely to facilitate ease of understanding and without any intention of limitation, several of the steps of the method 800 are illustrated in FIGS. 27A-27F.

At step 802, the distal end of the hollow casing 516 of the delivery device 500 and the remodeling device 10 are advanced laparoscopically into a patient's body cavity. In at least one embodiment, the remodeling device 10 is coupled with the lift system 512 of the delivery device 500 by way of the first and second mounting brackets 564, 566 of the lift system 512. Depending on the preference of the clinician performing the procedure, the lift system 512 may be positioned in an extended configuration or a collapsed configuration. In at least one embodiment of the method 800, at step 802 the remodeling device 10 coupled with the lift system 512 of the delivery device 500 are inserted laparoscopically into the patient's body through a 15 millimeter cannula under insufflation and fluoroscopic or direct camera control.

At step 804, after the distal end 518 of the hollow casing 516 is positioned properly within the patient's body cavity under fluoroscopic, direct camera control or otherwise, the first and second arms 502, 506 of the delivery device 500 are advanced simultaneously through the hollow casing 516, thereby moving the remodeling device 10 adjacent to the tissue of interest. In at least one embodiment, the first side 12A of the first component 12 is positioned adjacent to the anterior side of the targeted tissue or organ and the first side 16A of the second component 16 is positioned adjacent to the posterior side of the targeted tissue or organ. In addition, as shown in FIG. 27A, if the delivery device 500 was advanced through the abdominal cavity with the lift system 512 in a collapsed configuration, at step 804 the first lever 520 of the handle 514 of the delivery device 500 can be moved into the collapsed configuration, thereby causing the lift system 512 to move into the extended configuration. In this manner, the remodeling device 10 is now positioned to be placed round the targeted tissue.

After the components 12, 16 of the remodeling device 10 are sufficiently positioned relative to the targeted tissue or organ, the method 800 advances to step 808 for those embodiments of the remodeling device 10, 200, 300 comprising at least one moveable pin 22. At step 808 and as shown in FIG. 27B, the at least one pin 22 is deployed into the extended configuration through use of the handle 514 of the delivery device 500. For example, and without limitation, a clinician can deploy the at least one pin 22 of the remodeling device 10 by pulling the first component 582 of the second lever 524 of the handle 514 on the delivery device 500 in a proximal direction (as indicated by the arrows shown in FIGS. 20 and 27B). Due to the configuration of the delivery device 500, this manipulation of the first component 580 of the second lever 524 causes the latch mechanism 602 of the first coupling mechanism 545 to retract, which slides the pin actuating mechanism 612 of the first component 12 in a proximal direction and thereby moves the at least one pin 22 from the substantially retracted configuration to the substantially extended configuration. Accordingly, at step 808, the clinician deploys the at least one pin 22 of the remodeling device 10 into the substantially extended configuration via the handle 514 of the delivery device 500 that is located outside of the body.

At step 810, through use of the delivery device 500, the components 12, 16 of the remodeling device 10 are mechanically engaged through the targeted tissue. Specifically, after the components 12, 16 of the remodeling device 10 are properly positioned relative to the targeted tissue and the at least one pin 22 of the remodeling device 10 is deployed in steps 804 and 808, the first lever 520 of the delivery device 500 is moved from the collapsed position to the extended position as shown in FIG. 27C, thereby moving the lift system 512 from the expanded configuration to the collapsed configuration. The movement of the lift system 512 into the collapsed configuration ultimately causes the first and second components 12, 16 of the remodeling device 10 to simultaneously close down on and mechanically engage the underlying targeted tissue (not shown) disposed therebetween.

In the embodiments of the remodeling device 10, 200, 300 where either or both of the first sides 12A, 16A of the first and second components 12, 16 comprise one or more of pins 22 extending therefrom, the downward pressure exerted on the first and second components 12, 16 of the remodeling device 10 by the lift system 512 causes the one or more pins 22 to pierce the underlying targeted tissue. In fact, the concurrent movement of both components 12, 16 onto the targeted tissue driven by the lift system 512 facilitates the puncture of the underlying targeted tissue by the at least one pin 22 of the remodeling device 10. Particularly, in addition to the downward force provided by the lift system 512 as it moves into the collapsed configuration, the first side 12A, 16A of the opposite component 12, 16 from which the at least one pin 22 extends provides a support against which the distal end 44 of the pin 22 can be biased. After the distal end 44 of the at least one pin 22 has punctured the tissue disposed between the first and second components 12, 16, as illustrated in FIG. 27D, the distal end 44 locks into the engagement mechanism 28 disposed in the first side 12A, 16A of the opposite component 12, 16. In this manner, the first and second components 12, 16 are mechanically secured together.

Alternatively, the method 800 may be slightly modified at step 810 to accommodate the variations of embodiments of the remodeling device 300. In at least one embodiment of the method 800, at step 810 when the lift system 512 is moved into the collapsed configuration thereby driving the first and second components 12, 16 down onto the targeted tissue, the first component 12 is moved in a distal direction relative to the second component 16 through the use of an additional laparoscopic instrument inserted through a separate abdominal port. In this manner, the components of the latch mechanism 380 coupled with the proximal ends 13, 17 of the components 12, 16 are positioned in substantial alignment with each other and the stent-shaped lace component 376 is moved into the arched configuration. Thereafter, the latch mechanism 380 is secured through the use of the additional laparoscopic instrument referenced above, thereby forming an interior 70 and causing the components of the remodeling device 300 to compress the underlying targeted tissue positioned within the interior 70. However, as with the other embodiments of the remodeling device 10, 200, 300 described herein, while the targeted tissue does experience some compressional force exerted by the first and second components 12, 16 of the remodeling device 300, the majority of the compressional load is supported by the latch mechanism 380, potentially the lace component 260, and/or the at least one pins 22 of the remodeling device 300 (if present). Accordingly, irrespective of the differences between the various embodiments of the remodeling device 10, 200, 300 described herein, when applied to a targeted tissue, the remodeling device 10, 200, 300 is capable of effectively remodeling and/or providing support to the underlying tissue or organ in a desired manner without overly compressing the same.

As illustrated in FIGS. 27E and 27F, at step 812 the second lever 524 is again manipulated, which causes the first and second components 12, 16 to be released from the first and second mounting brackets 564, 567, respectively. For example and without limitation, a clinician can release the remodeling device 10 from the delivery device 500 by pulling the second component 582 of the second lever 524 in a proximal direction (as indicated by the arrow shown in FIG. 27E). Due to the configuration of the delivery device 500, this manipulation of the second component 582 of the second lever 524 causes the at least one posts 601 of the first and second coupling mechanisms 545, 567 to retract, thereby withdrawing the same from the receptacles 618 in the proximal ends 13, 17 of the first and second components 12, 16 and uncoupling from the same. Optionally, the first lever 520 of the handle 514 may also be manipulated by the clinician to move the lift system 512 from the collapsed configuration to the extended configuration in order to assist in uncoupling the first and second components 12, 16 of the remodeling device 10 from the first and second mounting brackets 564, 566 of the lift system 512. In this manner, at step 808 the remodeling device 10 is released from the delivery device 500 after the remodeling device 10 has been properly implanted on the targeted tissue. Due to the secure mechanical connection between the first and second components 12, 16 of the remodeling device 10, the remodeling device 10 can remain within the patient's body for as long as the remodeling or support treatment delivered thereby is desired.

After the remodeling device 10 is properly positioned on the targeted tissue and released from the delivery device 500, the delivery device 500 is withdrawn from the body cavity at step 814. In at least one embodiment, at step 814 the distal ends 504, 508 of the first and second arms 502, 506 and the lift system 512 are retracted into the interior of the hollow casing 516 and the distal end 518 of the hollow casing 516 is withdrawn from the body. It will be appreciated at the lift system 512 may be in either the extended configuration, the collapsed configuration or somewhere in between upon removal through the port, provided the diameter of the lift system 512 is sufficiently small to move through the port without damaging any tissue.

It will be understood that the remodeling device 10, 200, 300 can be delivered to a targeted tissue using either method 700 or method 800. However, for applications of the remodeling device 10, 200, 300 to certain tissues or organs, it may be necessary to provide additional leverage or force to assist in closing the first and second components 12, 16 around the tissue of interest and achieving a secure mechanical engagement between the two components 12, 16. Accordingly, in at least one embodiment, a clamp device 900 may be used in conjunction with the delivery device 500 to assist in the laparoscopic delivery of the remodeling device 10, 200, 300 to a targeted tissue.

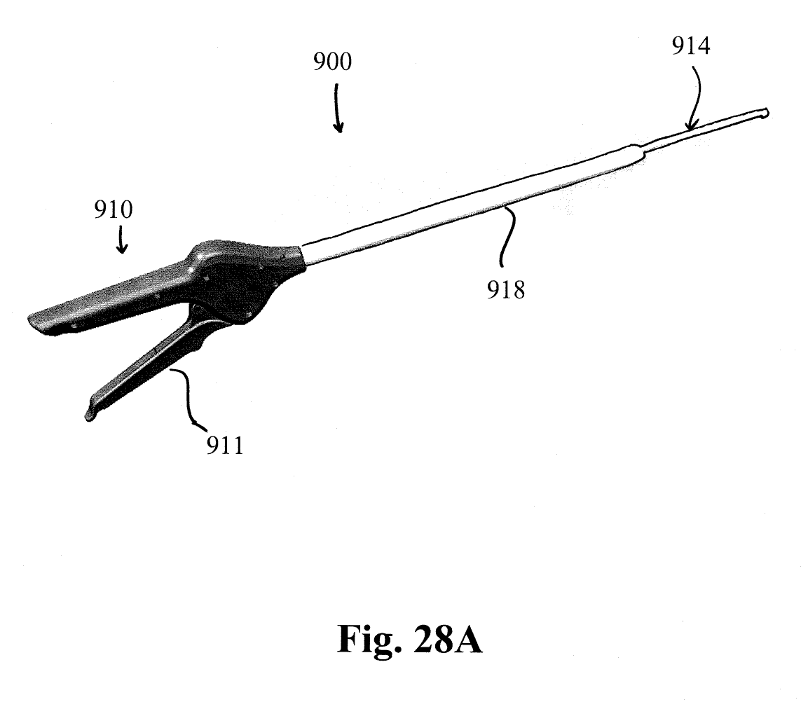

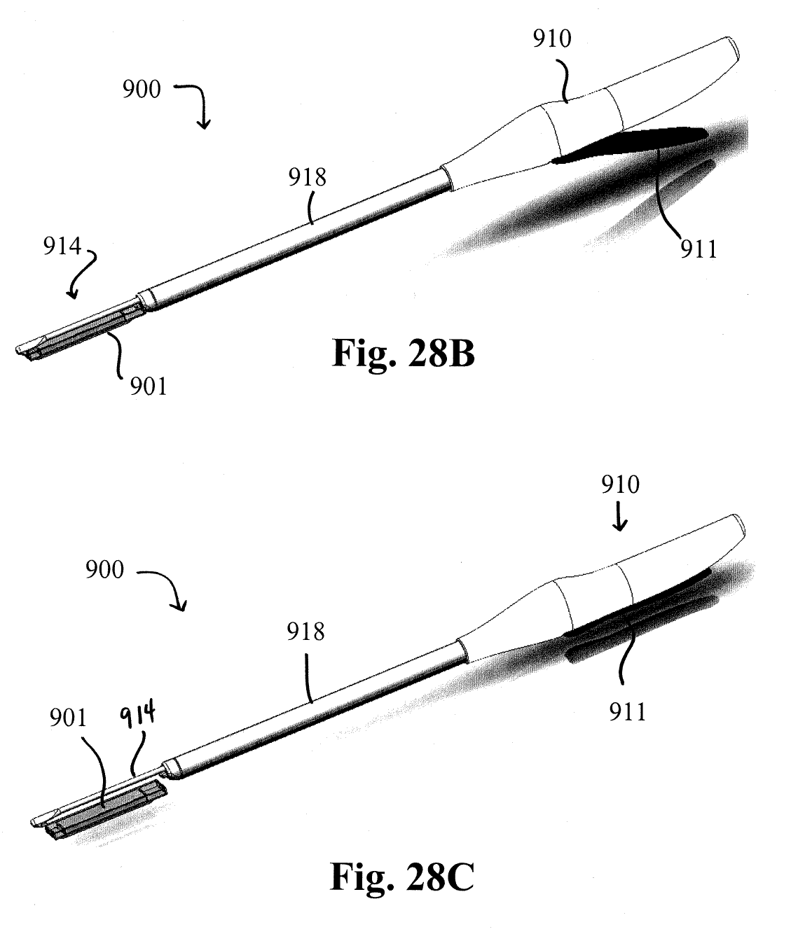

Now referring to FIGS. 28A-28C, at least one embodiment of a clamp device 900 is shown. The clamp device 900 may be used to deliver one or more stacking clamps 901 to the second sides 12B, 16B of the first and second components 12, 16 through a laparoscopic procedure. As shown in FIG. 28A, the clamp device 900 comprises a proximal end 910, a distal end 914, and an elongated body 918 extending therebetween. The proximal end 910 of the clamp device 900 comprises a handle 911 that is capable of moving between an extended configuration (see FIGS. 28A and 28B) and a compressed configuration (see FIG. 24C). In the at least one embodiment of the clamp device 900 shown in FIGS. 24A and 24B, the handle 911 is comprised of a standard lever.

The distal end 914 of the clamp device 900 comprises any coupling mechanism that is capable of securely coupling with a stacking clamp 901 (see FIG. 24B) and thereafter releasing the stacking clamp 901 upon movement of the handle 911 into the compressed configuration (see FIG. 24C). For example and without limitation, the distal end 914 of the clamp device 900 shown in FIGS. 24A-24C comprises a retractable hook configuration as is known in the art.

The stacking clamps 901 are configured similarly to the first and second components 12, 16 of the remodeling device 10; however, the stacking clamps 901 do not comprise an engagement mechanism 28 or one or more pins 22 and each stacking clamp 901 is configured to couple with the second side 12B, 16B of either the first or second components 12, 16. Each of the stacking clamps 901 may comprise a material suitable to resist corrosion, such as and without limitation, polyurethane, PTFE, silastic, titanium, stainless steel or any other material suitable for use in the medical arts that is corrosion resistant. Accordingly, each stacking clamp 901 can withstand chronic placement within a body without the risk of deterioration. In at least one embodiment, the stacking clamps 901 are comprised of ultra high density polyethylene.

When used in conjunction with the remodeling device 10, 200, 300, the stacking clamps 901 may be used in pairs. For example, a first stacking clamp 901 may be positioned adjacent to the first component 12 and a second stacking clamp 901 may be positioned adjacent to the second component 16. Furthermore, each of the stacking clamps 901 comprise a magnet disposed therein. It will be appreciated that the magnet may comprise the totality of the stacking clamp 901, or be disposed within or on the stacking clamp 901 in any fashion so long as an attractive magnetic force can be generated between the first and second stacking clamps 901 deployed in connection with the remodeling device 10, 200, 300.

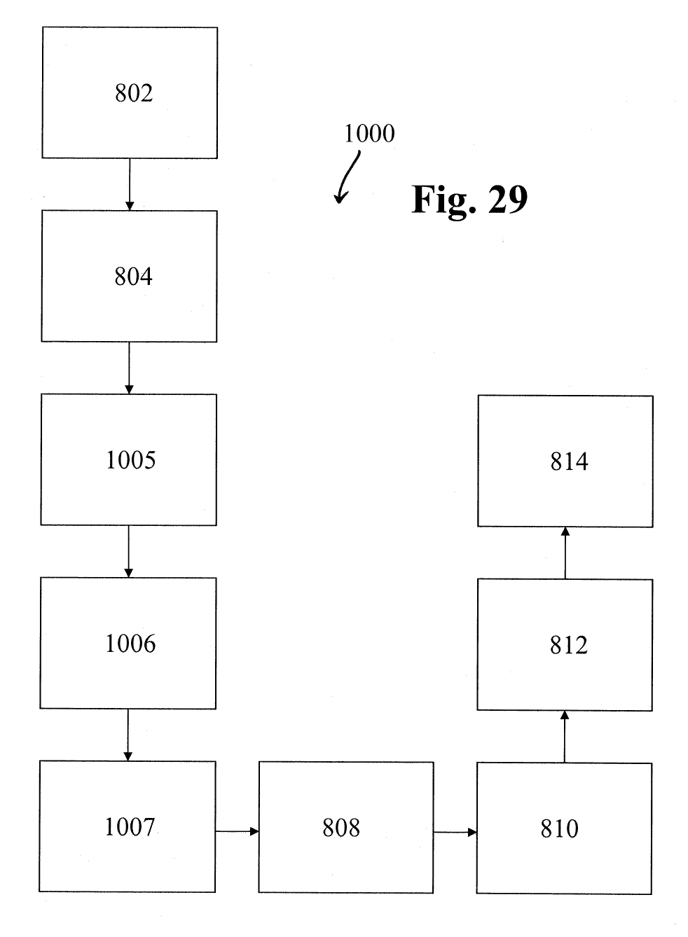

Referring now to FIG. 29, a flow chart of a method 1000 for laparoscopically delivering a remodeling device 10 through use of the delivery device 500 and the clamp device 900 is shown. It will be appreciated that the method 1000 builds upon the method 800 previously described herein and, as such, like reference numerals identify like steps between the two methods 800, 1000.

At step 802, the distal end of the hollow casing 516 of the delivery device 500 and the remodeling device 10 are advanced laparoscopically into a patient's body cavity through a first port. At step 804, under fluoroscopic, direct camera control or otherwise and after the distal end 518 of the hollow casing 516 is positioned properly within the patient's body cavity, the first and second arms 502, 506 of the delivery device 500 are advanced simultaneously through the hollow casing 516, thereby moving the remodeling device 10 adjacent to the tissue of interest. Specifically, at step 804, the first side 12A of the first component 12 is positioned adjacent to the anterior side of the targeted tissue or organ and the first side 16A of the second component 16 is positioned adjacent to the posterior side of the targeted tissue or organ.

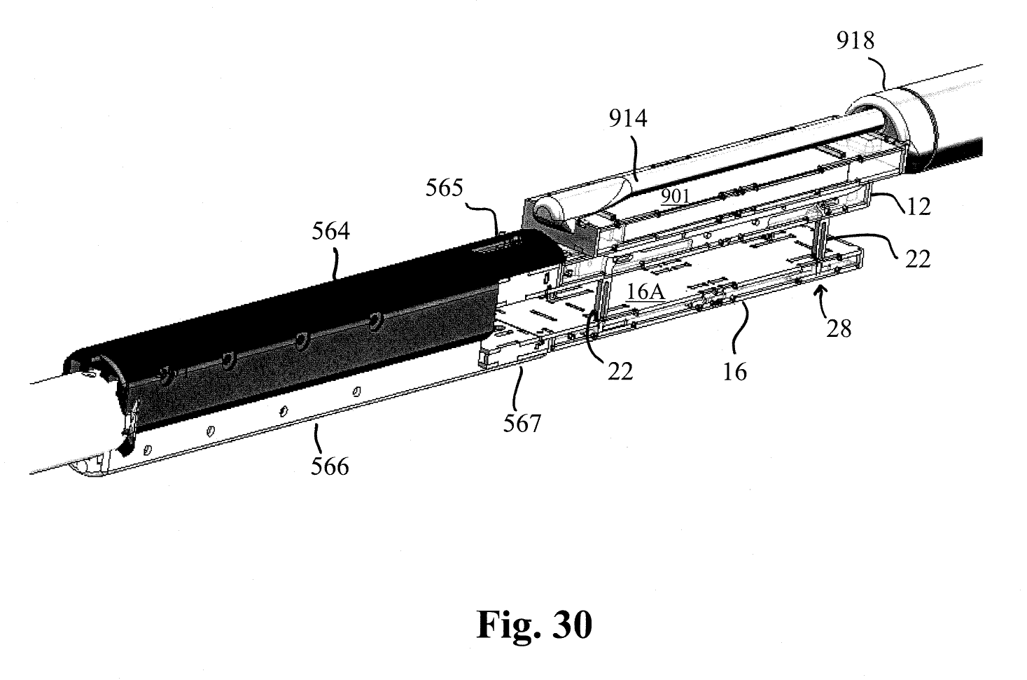

At step 1005, the distal end 914 of the clamp device 900 coupled with a first stacking clamp 901 is advanced laparoscopically into the patient's body cavity through a secondary 15 millimeter cannula, while sufflation is maintained. At step 1006, under fluoroscopic, direct camera control or otherwise, the distal end 914 of the clamp device 900, along with the first stacking clamp 901 coupled therewith, is advanced to the first component 12 of the remodeling device 10 that is coupled with the delivery device 500. In addition, as shown in FIG. 30, the clamp device 900 is used to maneuver the first stacking clamp 901 such that it is coupled with the second side 12B of the first component 12 of the remodeling device 10. Once the first stacking component 901 is properly positioned and secured to the first component 12 of the remodeling device 10, the handle 911 of the clamp device 900 is compressed, thereby releasing the first stacking clamp 901 from the distal end 914 of the clamp device 900.

After the first stacking component 901 is applied to the first component 12 of the remodeling device 10, the clamp device 900 is withdrawn from the body and a second stacking clamp 901 is removably coupled with the distal end 914 thereof. Accordingly, at step 1007, the second stacking clamp 901 is advanced laparoscopically into the patient's body cavity through the secondary 15 millimeter cannula and, under fluoroscopic, direct camera control or otherwise, the distal end 914 of the clamp device 900 is used to couple the second stacking clamp 901 with the second side 16B of the second component 16 of the remodeling device 10. After the second stacking component 901 is properly positioned and secured to the second component 16 of the remodeling device 10, the handle 911 of the clamp device 900 is compressed, thereby releasing the second stacking clamp 901 from the distal end 914 of the clamp device 900 and the clamp device 900 is withdrawn from the body cavity.

After the components 12, 16 are sufficiently positioned relative to the targeted tissue or organ and the first and second stacking clamps 901 have been secured to the same, the method 1000 advances to step 808. At step 808, the at least one pin 22 is deployed into the extended configuration through use of the delivery device 500 and, at step 810, the remodeling device 10 is mechanically engaged through the targeted tissue though use of the delivery device 500. The attractive magnetic force between the first and second stacking clamps 901 disposed on the first and second components 12, 16 strengthens the downward pressure exerted on the underlying tissue by the first and second components 12, 16, thereby facilitating the ease with which the at least one pin 22 of the remodeling device 10 can puncture the underlying tissue. In this manner, the inclusion of the first and second stacking clamps 901 may be used to assist the remodeling device 10, 200, 300 in remodeling thicker and more difficult tissues. Thereafter, the method 1000 proceeds to steps 812 and 814 as previously described in connection with the method 800.

The remodeling devices described herein and the delivery and clamp devices 500, 900 provide numerous benefits over the devices and systems of the prior art. The remodeling device 10, 200, 300 may be inserted laparoscopically and/or endoscopically, is minimally invasive, completely reversible and available for chronic placement without the risk of complications. Furthermore, use of the remodeling device 10, 200, 300 to treat and/or support a targeted tissue or organ produces a reduced amount of negative side effects than the procedures of the prior art for similar indications. In addition, the delivery device 500 allows the remodeling device 10, 200, 300 to be easily delivered in a procedure that takes as little as ten (10) minutes.

While the remodeling devices 10, 200, 300 are presented with respect to specific anatomy and treatment examples, as one of ordinary skill in the art would recognize, the remodeling devices 10, 200, 300 and the methods 700, 800 and 1000 may be expanded for use in treating any organ, limb or body structure that would benefit from reshaping, remodeling, or added support using reversible, easy to use, and easy to implement techniques for chronic placement.

The devices and methods have been presented in detail with reference to certain embodiments thereof, however, such embodiments are offered by way of non-limiting examples, as other versions are possible. It is anticipated that a variety of other modifications and changes will be apparent to those having ordinary skill in the art and that such modifications and changes are intended to be encompassed within the spirit and scope of the devices and methods as defined by the following claims.

1. A delivery device comprising:
a first arm and a second arm, both the first and second arms capable of slidable movement relative to each other;
a handle comprising a first activation system, the first activation system coupled with the first arm and the second arm and configured to slidably move the first arm and the second arm relative to each other; and
a lift system coupled with the first and second arms, the lift system comprising a first mounting bracket and a second mounting bracket and configured to move the first mounting bracket and the second mounting bracket between a substantially closed position and a substantially open position upon manipulation of the first activation system of the handle.

2. The delivery device of claim 1, wherein the lift system is capable of laparoscopic insertion into a body.

3. The delivery device of claim 1, wherein manipulation of the first activation system of the handle moves the first and second mounting brackets between the substantially closed position and the substantially open position in a parallel fashion.

4. The delivery device of claim 1, further comprising a hollow casing comprising a first end, a second end and an interior extending between the first and second ends, the interior of the hollow casing comprising the first and second arms slidably disposed therein.

5. The delivery device of claim 1, wherein the first activation system comprises a lever.

6. The delivery device of claim 1, wherein the first mounting bracket of the lift system comprises a first coupling mechanism configured to releasably couple with a first component of an instrument and the second mounting bracket of the lift system comprises a second coupling mechanism configured to releasably couple with a second component of the instrument.

7. The delivery device of claim 6, wherein the handle further comprises a second activation system coupled with the first and second coupling mechanisms, the second activation system configured to release the first and second components of the instrument from the first and second mounting brackets upon manipulation thereof.

8. The delivery device of claim 7, wherein the second activation system comprises a first cable extending between the second activation system and the first mounting bracket and a second cable extending between the second activation system and the second mounting bracket.

9. The delivery device of claim 7, wherein the second activation system further comprises a lever.

10. The delivery device of claim 8, wherein the first coupling mechanism comprises one or more posts configured to releasably couple with the first component of the instrument and the second coupling mechanism comprises one or more posts configured to releasably couple with the second component of the instrument.

11. The delivery device of claim 10, wherein both the first and second mounting brackets of the lift system each further comprises at least one slidable component.

12. The delivery device of claim 11, each of the one or more posts of the first coupling mechanism is mounted on one of the at least one slidable components of the first mounting bracket and each of the one or more posts of the second coupling mechanism is mounted on one of the at least one slidable components of the second mounting bracket.

13. The delivery device of claim 10, wherein the first cable of the second activation system is coupled with each of the one or more posts of the first coupling mechanism and the second cable of the second activation system is coupled with each of the one or more posts of the second coupling mechanism.

14. The delivery device of claim 6, wherein the first component of the instrument further comprises one or more pins moveable between a substantially retracted position and a substantially extended position, and the handle further comprises a third activation system coupled with the first coupling mechanism and configured to move the one or more pins of the first component of the instrument from the substantially retracted position to the substantially extended position.

15. The delivery device of claim 14, wherein the third activation system comprises at least one cable extending between the third activation system and the first mounting bracket.

16. The delivery device of claim 14, wherein the third activation system further comprises a lever.

17. The delivery device of claim 15, wherein the first coupling mechanism further comprises a latch mechanism configured to releasably couple with the first component of the instrument such that the latch mechanism is capable of interacting with and affecting the position of the one or more pins of the instrument.

18. The delivery device of claim 17, wherein the first mounting bracket comprises at least one slidable component and the latch mechanism is mounted on one of the at least one slidable components of the first mounting bracket.

19. The delivery device of claim 17, wherein the at least one cable of the third activation system is coupled with the latch mechanism of the first coupling mechanism.

20. The delivery device of claim 7, wherein the first component of the instrument further comprises one or more pins moveable between a substantially retracted position and a substantially extended position, and the handle further comprises a third activation system coupled with the first coupling mechanism and configured to move the one or more pins of the first component of the instrument from the substantially retracted position to the substantially extended position.

21. The delivery device of claim 20, wherein the second and third activation systems each comprise a lever and both of the levers of the second and third activation systems are hingedly coupled with a pivot point.

22. The delivery device of claim 21, wherein the levers of the second and third activation systems are independently moveable relative to each other around the pivot point.

23. A delivery device for facilitating the laparoscopic delivery of an instrument to a targeted tissue, the delivery device comprising:
a hollow casing comprising an elongated tube having a proximal end and a distal end connected by a body having a hollow interior, the distal end of the hollow casing capable of laparoscopic introduction into a body;
a first arm having a proximal end and a distal end, the first arm slidably disposed within the hollow casing;
a second arm having a proximal end and a distal end, the second arm slidably disposed within the hollow casing;
a lift system comprising a first set of shafts, a second set of shafts, a first mounting bracket and a second mounting bracket, the first set of shafts coupled with the distal end of the first arm and the first mounting bracket and the second set of shafts coupled with the distal end of the second arm and the second mounting bracket such that the first and second mounting brackets are pushed away or pulled toward each other in a parallel fashion in connection with the slidable movement of the first and second arms; and
a handle coupled with proximal end of the first arm, the proximal end of the second arm, and the proximal end of the hollow casing, the handle comprising a first activation system capable of slidably moving the first and second arms relative to each other.

24. The delivery device of claim 23, wherein the first and second mounting brackets are each configured to releasably couple with one or more components of the instrument.

25. The delivery device of claim 23, wherein the first activation system comprises at least one lever coupled with the proximal end of the first arm and the proximal end of the second arm.

26. The delivery device of claim 24, wherein the handle further comprises a second activation system in communication with the first and second mounting brackets of the lift system, the second activation system comprising a first element configured to uncouple the one or more components of the instrument from the first and second mounting brackets of the lift system upon manipulation thereof.

27. The delivery device of claim 26, wherein the second activation system of the lift system further comprises a second element configured to communicate with one or more pins of the one or more components of the instrument and move the one or more pins from a substantially retracted position to a substantially extended position upon manipulation thereof.

28. The delivery device of claim 27, wherein the first and second elements of the second activation system are hingedly moveable around a pivot point.

29. A method for delivering an instrument to a targeted tissue comprising the steps of:
providing a delivery device for facilitating the laparoscopic delivery of the instrument to a targeted tissue, the delivery device comprising:
a first arm and a second arm, both the first and second arms capable of slidable movement relative to each other,
a handle comprising a first activation system, the first activation system coupled with the first arm and the second arm and configured to slidably move the first arm and the second arm relative to each other, and
a lift system coupled with the first and second arms, the lift system comprising a first mounting bracket and a second mounting bracket and configured to move the first mounting bracket and the second mounting bracket between a substantially closed position and a substantially open position upon manipulation of the first activation system of the handle,
wherein the first mounting bracket is releasably coupled with a first component of an instrument and the second mounting bracket is releasably coupled with a second component of the instrument;
positioning the first component of the instrument adjacent to a first surface of a targeted tissue;
positioning the second component of the instrument adjacent to a second surface of a targeted tissue; and
closing the first and second components of the instrument around the targeted tissue in a parallel fashion through operation of the first activation system of the delivery device.

30. The method of claim 29, further comprising the step of moving the first and second mounting brackets to a substantially open position through operation of the first activation system.

31. The method of claim 29, wherein the steps of positioning the first component of the instrument adjacent to a first surface of a targeted tissue and positioning the second component of the instrument adjacent to a second surface of a targeted tissue occur simultaneously.

32. The method of claim 29, wherein:
the handle of the delivery device further comprises a second activation system coupled with the first and second mounting brackets, the second activation system configured to release the first and second components of the instrument from the first and second mounting brackets upon manipulation thereof; and
further comprising the step of releasing the first and second components of the instrument from the delivery device through operation of the second activation system of the handle.

33. The method of claim 29, further comprising the step of withdrawing the delivery device from the targeted tissue, thereby allowing the instrument to remain thereon.

34. The method of claim 32, wherein:
the second activation system further comprises a first cable extending between the second activation system and the first mounting bracket and a second cable extending between the second activation system and the second mounting bracket; and
the step of releasing the first and second components of the instrument from the delivery device through operation of the second activation system of the handle further comprises pulling the first and second cables through operation of the second activation system.

35. The method of claim 29, wherein:
the first mounting bracket further comprises one or more posts configured to releasably couple with the first component of the instrument and the second mounting bracket further comprises one or more posts configured to releasably couple with the second component of the instrument, and
the step of releasing the first and second components of the instrument from the delivery device through operation of the second activation system of the handle further comprises withdrawing the one or more posts of the first and second mounting brackets from the first and second components of the instrument, respectively, through operation of the second activation system.

36. The method of claim 35, wherein
the first component of the instrument further comprises one or more pins moveable between a substantially retracted position and a substantially extended position;
the second activation system is further configured to move the one or more pins of the first component of the instrument from the substantially retracted position to the substantially extended position; and
further comprising the step of moving the one or more pins of the first component of the instrument into the substantially extended position through operation of the second activation mechanism.

37. A kit for performing a medical procedure comprising:
an instrument comprising one or more components;
a delivery device comprising:
a first arm and a second arm, both the first and second arms capable of slidable movement relative to each other,
a handle comprising a first activation system, the first activation system coupled with the first arm and the second arm and configured to slidably move the first arm and the second arm relative to each other, and
a lift system coupled with the first and second arms, the lift system comprising a first mounting bracket and a second mounting bracket and configured to move the first mounting bracket and the second mounting bracket between a substantially closed position and a substantially open position in a parallel fashion upon manipulation of the first activation system of the handle; and
a fluoroscope.
